(19) United States
(12) Patent Application Publication
(10) Pub. No.: US 2012/0101257 A1
(43) Pub. Date: Apr. 26, 2012

(54) PROCESSES FOR PREPARING AMINO-SUBSTITUTED GAMMA-LACTAMS

(75) Inventors: William Lubell, Montreal (CA); Andrew Jamieson, Strathaven (GB); Nicolas Boutard, Angerville (FR); Luisa Ronga, Nola (IT); Daniel St-Cyr, Montreal (CA); Stephane Turcotte, St-Laurent (CA); Wang Chen, Guelph (CA)

(73) Assignee: UNIVERSITE DE MONTREAL, Montreal, QC (CA)

(21) Appl. No.: 13/257,592

(22) PCT Filed: Mar. 18, 2010
(86) PCT No.: PCT/CA10/00431

§ 371 Date: Dec. 7, 2011

Related U.S. Application Data
(60) Provisional application No. 61/202,637, filed on Mar. 20, 2009.

(51) Int. Cl. C07K 1/06; C07D 207/273; C07D 403/06; C07D 291/06; C07D 291/04

(52) U.S. Cl. 530/337; 544/2; 548/122; 548/468; 548/544; 548/550

(57) ABSTRACT

The present application describes general process for the preparation of amino-substituted gamma-lactams involving the reaction of synthons of the general Formulae (I) and (VI): with amines. The processes are amenable to solid phase synthetic techniques and therefore allow the efficient incorporation of amino-substituted gamma-lactams into a wide variety of structural scaffolds, including, in particular peptides.

### CROSS-REFERENCE TO RELATED APPLICATIONS

The present application claims priority on U.S. provisional application No. 61/202,637 filed on Mar. 20, 2009, which is hereby incorporated by reference in its entirety.

### FIELD OF THE APPLICATION

The present application relates to novel processes for the preparation of amino-substituted gamma-lactams, in particular to processes that are amenable to solid phase synthetic techniques. Also included are certain synthons used in the processes and amino-substituted gamma-lactams prepared using the processes of the application along with uses thereof.

### BACKGROUND OF THE APPLICATION

The secondary structure of proteins and peptides is an essential determinant for their bioactivity. Information concerning the biologically active conformation of a peptide is imperative for understanding function and may be gleaned by employing organic surrogates to mimic different secondary structures. Peptide mimics containing non-peptidic structural elements have been pursued to imitate the conformation and functional groups of the native peptide.^{i,ii,iii,iv,v,vi,vii,viii,ix} Technologies allowing the convenient and rapid introduction of such mimics into peptides constitute powerful tools for studying structure-activity relationships (SARs) and designing biologically active compounds in the drug discovery process.

Common methods for systematically scanning peptides for SAR studies include, alanine, enantiomeric amino acid, proline and N-alkyl amino acid scanning for respectively identifying the importance of side chains, configuration, conformation and hydrogen-bonding interactions.^{x} In addition to providing SAR information, such modifications have added benefits such as enhanced resistance to enzymatic degradation and improved duration of action.^{xi,xii} More recently, beta-amino acids have been used to systematically replace alpha-amino acid residues in a class I major histocompatibility complex (MHC)-restricted alloreactive T-cell epitope peptide to furnish analogues with improved affinity and enhanced resistance to proteolysis cleavage without impairing formation of MHC-peptide complexes.^{xiii} Macrocyclic lactam constraints have also been moved along a peptide chain by creating ring systems using side-chain to side-chain amide bond formation between Lys and Glu residues,^{xiv} as well as by using various backbone connections between two N-alkyl amide moieties.^{xv}

Systematic substitution of alpha-amino acid by aza-amino acid residues (in which the alpha-carbon is replaced by nitrogen) within a peptide has proven effective for identifying the importance of turn structures for biological activity.^{xvi} For example, the combination of aza-amino acid and indolizidinone amino acid scans of a calcitonin gene-related peptide 1 receptor antagonist revealed the importance of a type II′ beta-turn centered at the Gly³³-Pro³⁴ residues for antagonist activity.^{xvii} New solid-phase approaches for introducing constraints into peptide structure will expand the power of these methods for systematically studying the relevance of side-chain functionality and backbone conformation for activity.

alpha-Amino-gamma-lactam (Agl, 1, Scheme 1) has been utilized to constrain the backbone conformation of linear peptides to give beta-turn mimics.^{xviii,xix,xx} The first introduction of a so-called “Freidinger-Veber lactam” into luteinizing hormone-releasing hormone (LH-RH) resulted in analogue 3 (Scheme 1) with 8.9 times greater potency than the parent peptide in an in vitro pituitary cell culture.^{xviii} Such lactam containing peptides have since been used as chemotherapeutic and anticancer agents as well as modulators to study the renin-angiotensin-aldosterone (RAAS) system and serine protease inhibition.^{xxi}

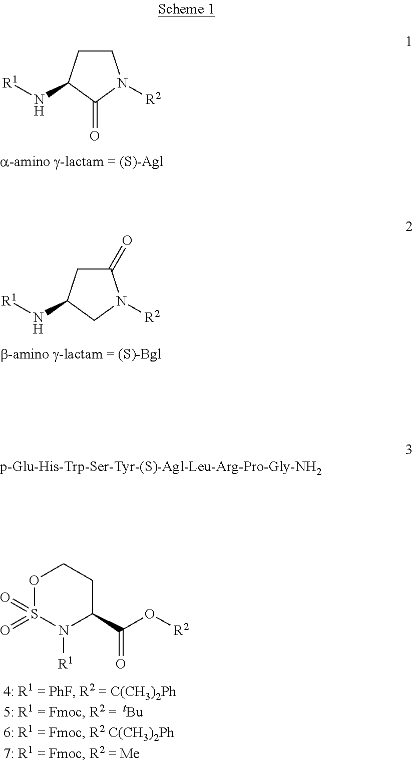

In comparison to its alpha-amino counterpart, beta-amino-gamma-lactam (Bgl, 2, Scheme 1) has been less frequently used to study peptide structures. The introduction Bgl in peptides was first reported during studies of the insulin potentiating and hypoglycemic activities of the fragment [6-13] of human growth hormone (hGH) in which replacement of Asp¹¹ by (S)-Bgl conserved biological activity and extended duration of action.^{xxii,xxiii,xxiv,xxv} In addition, Bgl peptide analogs have exhibited CCK-A receptor agonism with high affinity and selectivity,^{xxvi} as well as enhancement of the binding of [³H]N-propylnorapomorphine to dopamine receptors.^{xxvii}

Systematic scans of a peptide with alpha- and beta-amino-gamma-lactams may provide information concerning the structural requirements for biological activity by placing a constraint about the psi-dihedral angle. In this way, lactam scanning complements proline scanning in which the phi-dihedral angle is locked by a structural constraint.^{xxviii} Proline restricts the phi-torsion angle to values of −65±15°, yet adds conformational liberty about its N-terminal amide. In Agl (1), the gamma-lactam forces the C-terminal amide into the trans-orientation and restricts the psi-torsion angle to the range −125±10°,^{xxix} favoring a type II′ beta-turn geometry. Conformational analysis of Bgl (2) by NOESY-NMR studies in DMSO and molecular dynamics indicated that it too can stabilize a type II′ beta-turn conformation.^{xxv,xxx,xxxi}

Several synthetic strategies for the synthesis of Agl peptide mimics have been reported.^{xxi,xxxii,xxxiii,xxxiv,xxxv,xxxvi,xxxvii,xxxviii} Most are not suited for scanning peptides for biological activity because they require the synthesis of a dipeptide lactam in solution prior to incorporation into the peptide.^{xxxix,xl} At present, dipeptides bearing terminal five- and six-membered lactams have only been synthesized on solid phase either without stereocontrol by alkylation of Shiff-base protected imino esters with alpha,omega-dihaloalkanes,^{xl} or by the microwave assisted N-alkylation of resin-bound amino esters with fluorenylmethyl gamma-iodo-N-(Boc)amino butyrate and intramolecular lactam formation, albeit in low yield.^{xli} Recently, the seven member lactam Aia (4-amino-1,2,4,5-tetrahydro-indolo[2,3-c]-azepin-3-one) was introduced into biologically active peptides containing Trp residues by a solid-phase strategy featuring reductive amination using Fmoc-2′-formyl-tryptophan followed by lactam formation.^{xlii} Several solution phase methods for Bgl peptide synthesis have been described^{xxii, xxiii,xxiv}.

### SUMMARY OF THE APPLICATION

To study peptide structure-activity relationships, an efficient solid-phase methodology was developed for the synthesis of alpha- and beta-amino gamma-lactam peptides by regioselective ring opening of six- and five-member cyclic sulfamidates, followed by microwave irradiation to effect lactam formation in a one-step or two step-procedure. In the alkylation step on solid phase, peptide amine bis-alkylation was overcome by using a temporary silyl amine protection strategy. Lactam scanning was performed on both the growth hormone secretagogue GHRP-6 (HwAWfK) and the heptapeptide 101.10 (rytvela) giving the peptide lactams in good overall purified yields and high crude purity. This methodology has been successful on a wide variety of amino acid substrates making it generally applicable to the preparation of alpha- and beta-amino gamma-lactams derived from any primary amine.

Accordingly, in one aspect the present application includes a process for the preparation of alpha-amino-gamma-lactams or beta-amino-gamma-lactams comprising:
(a) reacting compounds of the Formula (I):

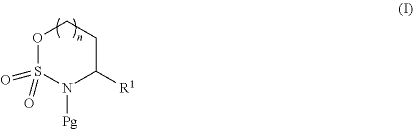

wherein Pg is a suitable protecting group,
n is 0 or 1,
when n is 1, R¹ is CO₂Bn, and
when n is 0, R¹ is selected from CH₂CO₂C₁₋₆alkyl and CH₂CO₂Bn,
with suitable amines of the Formula II:

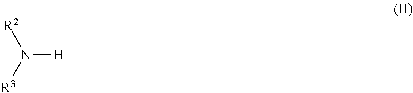

wherein R² is a hydrocarbon-based group comprising acyclic, cyclic, branched, unbranched, saturated, unsaturated and/or aromatic moieties that are unsubstituted or substituted with hetereomoieties, and
R³ is selected from H and a suitable protecting group,
under conditions to form compounds of the Formula (III):

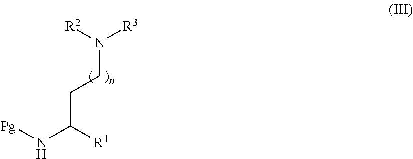

wherein Pg, R¹ and n are as defined in Formula (I), and
R² and R³ are as defined in Formula (II);
(b) if R³ is a suitable protecting group, removing R³;
(c) treating the compounds of the Formula (III), wherein R³ is H, under conditions to form alpha-amino-gamma-lactams or beta-amino-gamma-lactams of the Formula (IV):

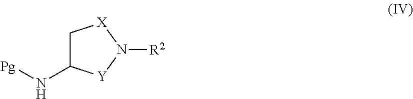

wherein Pg is as defined in Formula (I),
R² is as defined in Formula II, and
X is C(O) and Y is CH₂ when n in the compounds of Formula (I) is 0 and X is CH₂ and Y is C(O) when n in the compounds of Formula (I) is 1; and
(d) optionally removing Pg.

In an embodiment of the present application, the amines of Formula (II) are selected from one of the proteinogenic amino acids, suitably with side group functionalities in protected form, or an analog or derivative of one of the proteinogenic amino acids.

In another embodiment, the amines of Formula (II) are attached to a solid support such as those used in solid phase synthesis or combinatorial chemistry.

In a further embodiment of the present application, when R³ is H, (a) and (c) are performed in a single step, i.e. the conditions to form the compounds of the Formula (III) are sufficient to form the compounds of the Formula (IV) so that upon formation of the compound of the Formula (III), they are converted, in situ, into the compounds of the Formula (IV). In another embodiment of the application, the compounds of the Formula (III) are isolated and Pg is removed to provide a compound of the Formula (V):

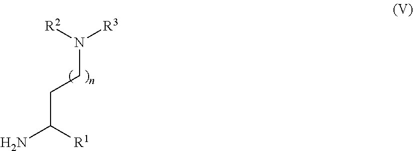

wherein R¹ and n are as defined in Formula (I) and R² and R³ are as defined in Formula (II).

In another aspect, the present application includes compounds of the Formula (I):

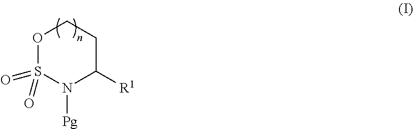

wherein Pg is a suitable protecting group;
n is 0 or 1;
when n is 1, R¹ is CO₂Bn; and
when n is 0, R¹ is selected from CH₂CO₂C₁₋₆alkyl and CH₂CO₂Bn.

Evaluation of the lactam analogs of GHRP-6 showed that peptides bearing Agl and Bgl at position 3 maintained affinity for the CD36 receptor and lost affinity towards the GHS-R1a receptor, in support of a beta-turn conformation for achieving affinity and selectivity of GHRP-6 analogs. The synthesis of six Agl-containing 101.10 analogs and the assessment of their efficacy in inhibiting IL-1 induced TF-1 cell proliferation, led to the discovery of a more efficient compound than 101.10 itself. Lactam scanning using the processes of the present application has thus proven useful for rapidly identifying peptide secondary structures required for affinity and biological activity.

The present application also includes compounds of Formula (IA):

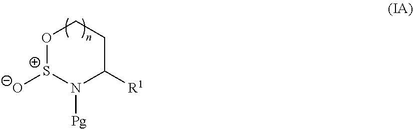

in which R¹, n and Pg are as previously mentioned.

The compounds of Formula (IA) are, for example, intermediate compounds of compounds of Formula (I)

In another aspect of the present application there is also included a process for the preparation of beta-hydroxy-alpha-amino-gamma-lactams comprising:

(a) reacting compounds of Formula (VI):

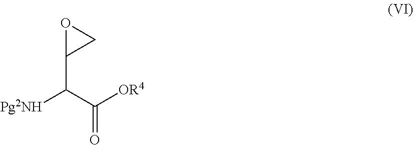

wherein Pg² is a suitable protecting group, and
R⁴ is selected from C₁₋₆alkyl and Bn,
with amines of the Formula (VII):

R⁵—NH₂ (VII)

wherein R⁵ is a hydrocarbon-based group comprising acyclic, cyclic, branched, unbranched, saturated, unsaturated and/or aromatic moieties that are unsubstituted or substituted with hetereomoieties,
under conditions to form compounds of the Formula (VIII):

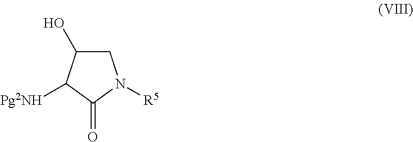

wherein Pg² is as defined in Formula (VI) and R⁵ is as defined in Formula (VII); and

(b) optionally removing Pg².

In an embodiment of the present application, the amines of Formula (VII) are selected from one of the proteinogenic amino acids, suitably with side group functionalities in protected form, or an analog or derivative of one of the proteinogenic amino acids.

In another embodiment, the amines of Formula (VII) are attached to a solid support such as those used in solid phase synthesis or combinatorial chemistry.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The application will now be described in relation to the drawings in which:

FIG. 1 is a graph showing the inhibition of IL-1 induced TF-1 cell proliferation by peptides 54-59.

FIG. 2 is a graph showing the inhibition of IL-1 induced TF-1 cell proliferation by compounds 75-78.

### DETAILED DESCRIPTION OF THE APPLICATION

### (I) Definitions

Unless otherwise specified, the following definitions apply to all aspects and embodiments of the present application.

The term “hetereomoiety” or “hetereomoieties” as used herein means a functional grouping comprising at least one non-carbon or non-hydrogen atom.

The term “proteinogenic amino acid” refers to the standard alpha-amino acids that are the building blocks of proteins, and are therefore encoded by the genetic code, and include

| | 3-Letter | 1-Letter |
|---|---|---|
| Name | Abbreviation | Abbreviation |
| Alanine | Ala | A |
| Arganine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term analog of derivative of a proteinogenic amino acid refers to alpha-amino acids that are derived from one of the proteinogenic amino acid, but in which one or more of the functional groupings has been replaced, deleted, modified or added to. This includes alpha-amino acids that are not typically encoded by the genetic code but are also found in proteins in the body and include, for example, selenocysteine and pyrrolysine. Also included are N-methyl amino acids, beta-amino acids, fluoro-substituted amino acids and the like.

The abbreviation “Fmoc” means fluorenylmethoxycarbonyl”.

The abbreviation “Bn” means benzyl.

As used herein, the term “alkyl” whether used alone or as part of a substituent group, includes straight and branched chains. For example, alkyl groups include methyl, ethyl, propyl, isopropyl, butyl, isobutyl, sec-butyl, t-butyl, pentyl and the like. Unless otherwise noted, “C₁₋₆” when used with alkyl means a straight or branched carbon chain composition of 1, 2, 3, 4, 5 or 6 carbon atoms.

The term “aryl” as used herein refers to a cyclic or polycyclic carbocyclic ring systems containing at least one aromatic ring. In an embodiment, an aryl group is phenyl or naphthyl.

The terms “protective group” or “protecting group” or “Pg” or the like as used herein refer to a chemical moiety which protects or masks a reactive portion of a molecule to prevent side reactions in those reactive portions of the molecule, while manipulating or reacting a different portion of the molecule. After the manipulation or reaction is complete, the protection group is typically removed under conditions that do not destroy or decompose the molecule. Many conventional protecting groups, and methods for their addition to and removal from a molecule, are known in the art for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. These include, but are not limited to, benzyloxy carbonyl (Cbz), t-butoxycarbonyl (t-Boc), tosyl (Ts), mesityl (Ms), trimethylsilyl (TMS), t-butyldimethylsilyl (TBDMS), t-butyldiphenylsilyl (TBDPS), triflate (Tf), Bn, allyl, fluorenylmethoxy carbonyl (Fmoc), C₁₋₁₆acyl, silyl, acetal and the like.

One skilled in the art will recognize that wherein a reaction step of the present application may be carried out in a variety of solvents or solvent systems, said reaction step may also be carried out in a mixture of the suitable solvents or solvent systems.

The term “pharmaceutically acceptable” means compatible with the treatment of animals, in particular, humans.

The term “solvate” as used herein means a compound, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In general, prodrugs will be functional derivatives of a compound which are readily convertible in vivo into the compound from which it is notionally derived. In an embodiment of the application, prodrugs are conventional esters formed with available hydroxy or amino groups. For example, an available OH or NH group in a compound is acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In further embodiments, the prodrugs are those in which one or more of the hydroxy groups in the compound is masked as groups which can be converted to hydroxy groups in vivo. In a further embodiment, prodrugs of the compounds are conventional imines, oximes, acetals, enol esters, oxazolidines or thiazolidines formed with the available aldehyde group. Conventional procedures for the selection and preparation of suitable prodrugs are described, for example, in “Design of Prodrugs” ed. H. Bundgaard, Elsevier, 1985 or are known to a person skilled in the art.

The term “pharmaceutically acceptable salt” means an acid addition salt or a basic addition salt which is suitable for or compatible with the treatment of patients.

The term “pharmaceutically acceptable acid addition salt” as used herein means any non-toxic organic or inorganic salt of any base compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acid compound. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### (II) Processes of the Application

To enhance the application of amino lactams in peptide science and medicinal chemistry, a general strategy for the synthesis of compounds bearing alpha-amino-gamma-lactams (Agl) and beta-amino-gamma-lactams (Bgl) has been developed featuring the regiospecific ring-opening of enantiomerically pure five- and six-member cyclic sulfamidates, followed by microwave assisted lactam formation. Further, a general strategy using standard Fmoc-based peptide solid phase synthesis has been developed for the incorporation of Agl and Bgl into peptides.

Five-member cyclic sulfamidates (2,2-dioxo-1,2,3-oxathiazolidines) and six-member cyclic sulfamidates (2,2-dioxo-1,2,3-oxathiazinanes) have emerged as effective amino alcohol-derived bis-electrophiles for the synthesis of amino acid and heterocyclic products, in part, because the sulfamidate simultaneously activates the hydroxyl group and protects the amine moiety.^{xliv,xlv} In particular, six-member cyclic sulfamidates, such as (4S)-cumyl-2,2-dioxo-3-N-PhF-1,2,3-oxathiazinane-4-carboxylate (4, PhF=9-(9-phenylfluorenyl, Scheme 1), have served in the solution-phase synthesis of lactam bridged dipeptides,^{xxxvii,xlvi} providing access to a greater variety of C-terminal amino acid residues; however, application of the products in peptide synthesis necessitated switching amine protection. (4S)-tert-Butyl-2,2-dioxo-3-N-Fmoc-1,2,3-oxathiazinane-4-carboxylate (5) (Scheme 1) was later employed in the solution-phase alpha-amino lactam synthesis of Fmoc-Agl-Trp, which was later introduced by solid-phase chemistry, as a replacement of the Ala-Trp dipeptide unit, into the growth hormone secretagogue peptide GHRP-6.^{xlvi,xlvii} With construction of the alpha-amino gamma-lactam onto a resin-bound peptide as the next goal, (4S)-cumyl- and (4S)-methyl-2,2-dioxo-3-N-Fmoc-1,2,3-oxathiazinane-4-carboxylates (5 and 7, Scheme 1) were respectively employed in the solid-phase syntheses of analogs of enkephalin^{xlvii} and LHRH.^{xlviii}

In these earlier studies, the ester type was found to influence significantly the reactivity of the 2,2-dioxo-3-N-Fmoc-1,2,3-oxathiazinane-4-carboxylate in both the peptide alkylation and lactam formation steps. A detailed study of the ester protective group was performed and a practical solid-phase protocol for making alpha-amino lactam peptides using a benzyl ester has now been developed and employed in lactam scanning of GHRP-6 as well as the peptide allosteric modulator of the IL-1 receptor 101.10^{xlix}. Moreover, by employing a five-member cyclic sulfamidate in a similar protocol, beta-amino lactams have also been effectively introduced into peptides on solid phase.

Accordingly, in one aspect the present application includes a process for the preparation of alpha-amino-gamma-lactams or beta-amino-gamma-lactams comprising:
(a) reacting compounds of the Formula (I):

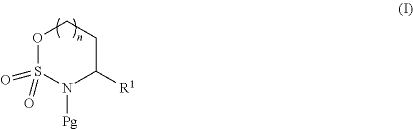

wherein Pg is a suitable protecting group,
n is 0 or 1,
when n is 1, R¹ is CO₂Bn, and
when n is 0, R¹ is selected from CH₂CO₂C₁₋₆alkyl and CH₂CO₂Bn,
with suitable amines of the Formula II:

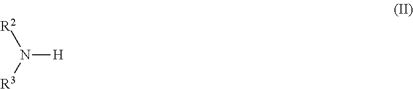

wherein R² is a hydrocarbon-based group comprising acyclic, cyclic, branched, unbranched, saturated, unsaturated and/or aromatic moieties that are unsubstituted or substituted with hetereomoieties, and
R³ is selected from H and a suitable protecting group,
under conditions to form compounds of the Formula (III):

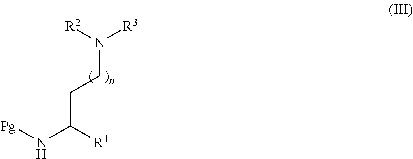

wherein Pg, R¹ and n are as defined in Formula (I), and
R² and R³ are as defined in Formula (II);
(b) if R³ is a suitable protecting group, removing R³;
(c) treating the compounds of the Formula (III), wherein R³ is H, under conditions to form alpha-amino-gamma-lactams or beta-amino-gamma-lactams of the Formula (IV):

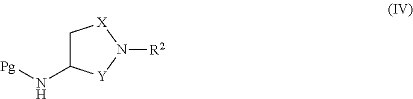

wherein Pg is as defined in Formula (I),
R² is as defined in Formula II, and
X is C(O) and Y is CH₂ when n in the compounds of Formula (I) is 0 and X is CH₂ and Y is C(O) when n in the compounds of Formula (I) is 1; and
(d) optionally removing Pg.

The process of the present application has been shown to work using a wide variety of amines of Formula (II), including, for example, amines wherein R² and/or R³ comprises aliphatic, aromatic, polar, heteromoiety functioanlized and sterically hindered groups. Accordingly, the process of the present application is application to any amine of Formula (II).

In an embodiment of the present application, the amines of Formula (II) are selected from one of the proteinogenic amino acids, suitably with side group functionalities in protected form. In another embodiment of the application, the amines of Formula (II) are an analog or derivative of one of the proteinogenic amino acids.

In a further embodiment of the present application, the process further comprises, following (c), reacting the compounds of the Formula (III) to replace any protecting groups that were present on any of the functional groups in R² that were removed during (a) and/or (c). Such “capping” reactions are a particular aspect of the present application, when the compounds of Formula (III) and/or (IV) are to be used as starting materials for further chemical transformations.

In another embodiment, the amines of Formula (II) are attached to a solid support such as those used in solid phase synthesis or combinatorial chemistry. In an embodiment, the solid support is any solid support used in solid phase peptide synthesis, such as, but not limited to, the Rink amide resin and the Rink amide MBHA resin. In an embodiment, the solid support is any solid support used in combinatorial synthesis, such as, but not limited to, miniaturized devices that contain both a functionalized solid phase support and a unique tag identifier or cylindrical devices that are modular in nature and based on a structure of a rigid polymeric support beneath a grafted mobile phase.

In an embodiment of the disclosure, the conditions to form compounds of the Formula (III) comprise reacting excess amounts of the compounds of Formula I with the amines of Formula (II) in a suitable organic solvent, for example tetrahydrofuran, at temperatures in the range of about 20° C. to about 70° C., for about 1 hour to about 48 hours. In a further embodiment, the conditions to form compounds of the Formula (III) further comprise reacting excess amounts of the compounds of Formula I with the amines of Formula (II) in presence of microwave irradiation. The use of microwave irradiation allows the use of shorter reactions times.

In an embodiment of the present application, R³ is a silyl protecting group, for example, trimethylsilyl, that is readily removed immediately following (a). In an embodiment of the application the use of a protecting group reduces the amount of bisalkylation of the amines of Formula (II). Alternatively, when R³ is H, the conditions to form compounds of the Formula (III) comprise reacting the compounds of the Formula (I) with the compounds of the Formula (II) in the presence 1 equivalent or less of a non-nucleophilic base, such as a tertiary amine base such as diisopropylethylamine (DIEA). This latter method also reduces the amount of bisalkylation of the amines of Formula (II).

In an embodiment of the present application, the conditions to form compounds of the Formula (IV) comprise treating the compounds of the Formula (III) wherein R³ is H in a suitable organic solvent, such as dimethylsulfoxide (DMSO) or dimethylformamide (DMF) in the presence of microwave irradiation at a temperature of about 60° C. to about 120° C., suitably about 70° C. to about 100° C., more suitably about 80° C., for about 10 minutes to about 24 hours, suitably about 30 minutes to about 10 hours. In an embodiment of the present application, the conditions to form compounds of the Formula (IV) further comprise the addition of an acid, such as a 1% acetic acid solution, to the reaction solvent, in particular DMSO. In another embodiment, the conditions to form compounds of the Formula (IV) further comprise the addition of water to the reaction solvent.

In a further embodiment of the present application, when R³ is H, (a) and (c) are performed in a single step, i.e. the conditions to form the compounds of the Formula (III) are sufficient to form the compounds of the Formula (IV) so that upon formation of the compound of the Formula (III) it is converted, in situ, into the compounds of the Formula (IV). In this embodiment, the process if performed in the presence of microwave irradiation.

A person skilled in the art would appreciate that reaction temperatures and time for the process of the application will vary depending on a number of factors, such as the structures of the starting materials and the scale of the reaction, and could adjust the reaction conditions (i.e. solvent, temperature and reaction time) accordingly to optimize yields.

In another embodiment of the application, the compounds of the Formula (III) are isolated and Pg is removed to provide a compound of the Formula (V):

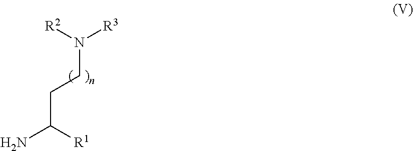

wherein R¹ and n are as defined in Formula (I) and R² and R³ are as defined in Formula (II). (V)

It is an aspect of the present application that the process is performed on a solid support using standard peptide synthesis procedures to insert one or more alpha-amino-gamma-lactam and/or beta-amino-gamma-lactam groups into a peptide. It is an embodiment of the application that the standard peptide synthesis procedures are Fmoc-based, i.e. Pg is Fmoc. In an embodiment, the general procedure for the use of the process of the present disclosure in peptide synthesis is shown in Scheme 2:

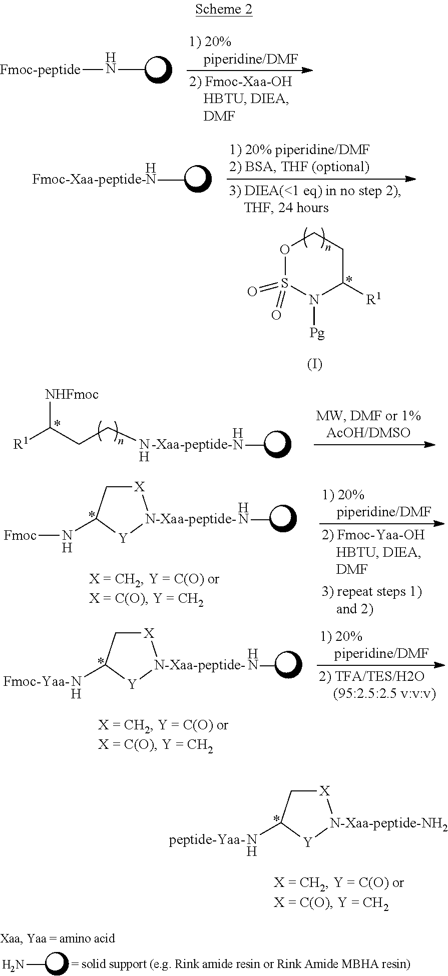

As shown in Scheme 2, standard conditions for solid-phase peptide synthesis with Fmoc protection is first used to synthesize the peptide chain on a resin, such as a Rink amide resin. When the appropriate residue for lactam formation is reached, the N-terminal Fmoc group is removed and the resin is dried, for example in vacuo. In an embodiment, the N-terminal amine is protected, for example, by reaction with BSA, alkylated with a compound of the Formula I and converted to the cyclic lactam. The latter two transformations are done in one or two steps. In an embodiment, the alkylation and/or lactam formation reactions are done in the presence of microwave irradiation. In an alternate embodiment, the N-terminal amine is not protected, but instead reacted with a compound of Formula I in the presence of 1 or less equivalents of a non-nucleophilic amine base, such as DIEA. Once the lactam is installed, the resulting peptide is either cleaved from the resin and protecting groups removed or, the protecting group is removed and further amino acid residues are added using standard coupling conditions. It is an embodiment, that the compounds obtained by alkylating compounds of Formula I, for example those shown on line two of Scheme 2 are not subjected to lactamization conditions and are used to prepare peptides comprising, when n is 1, a benzyl-2-aminobut-4-yloate moiety (Bab).

In another aspect of the present disclosure, compounds of Formula (II) are either commercially available or are prepared using methods known in the art.

In another aspect of the present application there is also included a process for the preparation of beta-hydroxy-alpha-amino-gamma-lactams comprising:

(a) reacting compounds of Formula (VI):

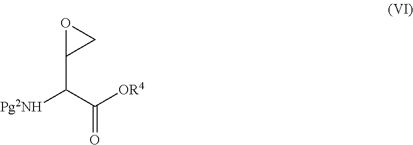

wherein Pg² is a suitable protecting group, and
R⁴ is selected from C₁₋₆alkyl and Bn,
with amines of the Formula (VII):

R⁵—NH₂ (VII)

wherein R⁵ is a hydrocarbon-based group comprising acyclic, cyclic, branched, unbranched, saturated, unsaturated and/or aromatic moieties that are unsubstituted or substituted with hetereomoieties,
under conditions to form compounds of the Formula (VIII):

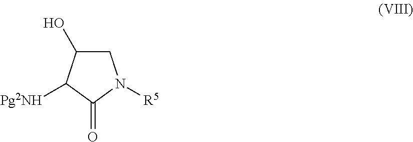

wherein Pg² is as defined in Formula (VI) and R⁵ is as defined in Formula (VII); and

(b) optionally removing Pg².

In an embodiment of the present application, the amines of Formula (VII) are selected from one of the proteinogenic amino acids, suitably with side group functionalities in protected form, or an analog or derivative of one of the proteinogenic amino acids. In another embodiment, (b) further comprises removing any side group protecting groups on R⁵.

In another embodiment, the amines of Formula (VII) are attached to a solid support such as those used in solid phase synthesis or combinatorial chemistry.

In a further embodiment, Pg² is Fmoc.

The compounds of Formula VI have at least one chiral or asymmetric centre. Where the compounds possess one asymmetric centre, they exist as enantiomers. Where the compounds possess more than one asymmetric centre, they exist as diastereomers. It is to be understood that all such isomers and mixtures thereof in any proportion are encompassed within the scope of the present application. It is to be understood that while the stereochemistry of the compounds may be as provided for in any given compound listed herein, such compounds may also contain certain amounts (e.g. less than 20%, preferably less than 10%, more preferably less than 5%) of compounds of the present application having alternate stereochemistry.

In another aspect of the present disclosure, compounds of Formulae (VI) and (VII) are either commercially available or are prepared using methods known in the art. In an embodiment of the application, compounds of Formula (VI) are prepared using methods described.

### Compounds of the Application

In another aspect, the present application includes compounds of the Formula (I):

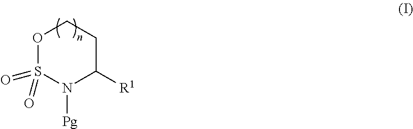

wherein Pg is a suitable protecting group,

n is 0 or 1,

when n is 1, R¹ is CO₂Bn, and

when n is 0, R¹ is selected from CH₂CO₂C₁₋₆alkyl and CH₂CO₂Bn.

In an embodiment of the application, Pg is Fmoc. In a further embodiment, n is 1 and R¹ is CO₂Bn. In another embodiment, n is 0 and R¹ is CH₂CO₂Me.

The compounds of Formula I have at least one chiral or asymmetric centre. Where the compounds possess one asymmetric centre, they exist as enantiomers. Where the compounds possess more than one asymmetric centre, they exist as diastereomers. It is to be understood that all such isomers and mixtures thereof in any proportion are encompassed within the scope of the present application. It is to be understood that while the stereochemistry of the compounds may be as provided for in any given compound listed herein, such compounds may also contain certain amounts (e.g. less than 20%, preferably less than 10%, more preferably less than 5%) of compounds of the present application having alternate stereochemistry.

In an aspect of the present application, the compounds of Formula (I) wherein n is 1 are synthesized from methionine as a chiral educt. In an embodiment, homoserine is prepared from methionine by S-alkylation followed by intramolecular displacement to form homoserine lactone and subsequent hydrolytic ring opening using a known procedure.^{li} In a further embodiment, N-Fmoc protection and benzyl ester formation is achieved by acylation with N-(9-fluorenyl-methoxycarbonyloxy) succinimide (Fmoc-OSu) and alkylation of the resulting N-Fmoc-homoserine with, for example, benzyl bromide, is achieved using known procedures. Benzyl N-(Fmoc)-homoserine esters are susceptible to lactonization, therefore, in an embodiment, they are purified immediately by chromatography and stored at reduced temperature, for example −4° C. In a further embodiment, cyclic sulfamidites (2-oxo-1,2,3-oxathiazinanes) are produced, respectively, as 2:1 and 4:1 mixtures of diastereomers, by treating N-Fmoc-homoserine esters with a premixed solution of thionyl chloride and imidazole in a dilute THF solution in order to minimize formation of sulfite dimer^{lii} In another embodiment, subsequent oxidation to 2,2-dioxo-1,2,3-oxathiazinanes (compounds of Formula (I), wherein n is 1) is preformed using catalytic ruthenium(III) trichloride hydrate and sodium metaperiodate.^{liii} Using L-methionine as the starting material provides the corresponding (4R)-enantiomer of the compound of Formula (I), wherein n is 1, whereas the corresponding (4S)-enantiomer is prepared from D-methionine.

A study by ¹H NMR spectroscopy of the relative stability of compound of Formula (I), wherein n is 1, with the corresponding methyl ester revealed that both compounds were stable as solutions in CDCl₃ for 4 weeks. However, when stored neat at 4° C. the methyl ester decomposed totally within 8 days as observed by TLC (50% EtOAc/Hexanes). The crystalline benzyl ester was stable at 4° C. for 3 weeks before decomposition began to be observed by TLC (complete decomposition required longer than 2 months). The limited stability of the methyl ester relative to its benzyl ester counterpart made the latter the choice of reagent for use in further studies.

In another aspect of the present disclosure, the compounds of Formula I, wherein n is 0, are prepared form aspartic acid as chiral educt. In an embodiment, beta-methyl N-(Fmoc)aspartate, prepared using known methods^{liv,lv} is converted to the corresponding alcohol by activation as a mixed anhydride, using, for example iso-butylchloroformate and N-methylmorpholine, and in situ reduction, suitably at low temperature (for example, from −78° C. to −20° C.) to prevent lactone and diol formation.^{lvi} In an embodiment, the compounds of Formula I are produced by convertion of the alcohol in a two-step process as described for the preparation of its six-member counterpart above, without purification of the sulfamidite intermediate.

In another aspect of the present application, there is included a compound as shown in any one of Tables 1-10 or a pharmaceutically acceptable salt, solvate and prodrug thereof.

The present application also includes all uses, including as a medicament or as research, drug discovery and/or diagnostic tools, of the compounds shown in any one of Tables 1-10 as well as compositions, including pharmaceutical compositions comprising these compounds and a carrier, suitably a pharmaceutically acceptable carrier.

### EXAMPLES

### General Methods

### For Examples 1-30

Reagents and starting materials were obtained from commercial sources and used as received. L-Homoserine was purchased from Novabiochem® (EMD Bioscience Inc., San Diego). Fmoc amino acids were purchased from Novabiochem® (EMD Bioscience Inc., San Diego) or GL Biochem (Shangai) Ltd. Reactions requiring anhydrous conditions were performed under an atmosphere of dry argon; glassware, syringes and needles were flame dried immediately prior to use and allowed to cool either in a desiccator or under an atmosphere of dry argon; liquid reagents, solutions or solvents were added via syringe through rubber septa. Anhydrous solvents (THF, MeCN, DCM and DMF) were obtained from a Seca Solvent Filtration System (GlassContour™ Laguna Beach, Calif.). Triethylamine (Et₃N) and diisopropylethylamine (DIEA) were distilled first from ninhydrin, then from calcium hydride. The removal of solvents in vacuo was achieved using both a Büchi™ rotary evaporator (bath temperatures up to 40° C.) at a pressure of either 15 mm Hg (water aspirator) or 0.1 mm Hg (oil pump), as appropriate, and a high vacuum line at room temperature. Flash column chromatography^{lvii} was carried out using SiliaFlash®F60 silica (Silicycle, Quebec City). The crude material was applied to the column as a solution in DCM. Glass backed plates pre-coated with silica gel (SiliaPlate TLC Extra Hard Layer 60 Angstrom F-254, Silicycle, Quebec City) were used for thin layer chromatography and were visualized by UV fluorescence or staining with iodine, ninhydrin, phosphomolybdic acid, KMnO₄, or Ceric Ammonium Molybdate solution and heating.

¹H NMR and ¹³C NMR spectra were recorded on a Bruker AV300, ARX 400 or AV700 Ultrashield spectrometer with chemical shift values in ppm relative to residual chloroform (d_{H} 7.26 & d_{C} 77.2), acetone (d_{H} 2.05 & d_{C} 29.84), DMSO (d_{H} 2.50 & d_{C} 39.52) or deuterium oxide (d_{H} 4.79) as standard. J values are given in Hz. Other abbreviations used are: s (singlet), d (doublet), t (triplet), q (quartet), m (multiplet) and br (broad). Assignments of ¹H NMR and ¹³C NMR signals were made possible, using COSY, HMQC, HMBC, NOESY and DEPT experiments. Additional peaks in ¹³C spectra due to Fmoc rotomerism are indicated in square brackets [ ]. The unambiguous assignment of J values was made possible by simultaneously decoupling several interfering signals. Infra-red spectra were recorded on a Perkin-Elmer Spectrum One spectrometer. Mass spectral data and high-resolution mass spectrometry (HRMS) (electrospray ionization (ESI)) were obtained from the Université de Montreál Mass Spectrometry Facility. Optical rotations were determined for solutions by irradiating with the sodium D line (I=589 nm) using a Perkin Elmer 341 polarimeter. Specific rotation, [a]_{D} values are given in units 10⁻¹degcm²g⁻¹. Melting points were determined on a Gallenkamp digital melting point apparatus and are uncorrected. Microwave assisted reactions were preformed in a Biotage Initiator microwave apparatus (Biotage AB, Uppsala, Sweden) in glass Biotage reaction vessels containing a magnetic stirrer bar and sealed with a septum cap. Analytical RP-HPLC analyses were preformed on a Gemini™ (Phenomenex® Inc., Torrance, Calif.) column (150 mm×4.6 mm, 5 mm, C₁₈) with a flow rate of 0.5 mL/min using a linear gradient of water (0.1% formic acid) and acetonitrile (0.1% formic acid). Retention times (t_{R}) from analytical RP-HPLC are reported in minutes. Peptides were purified with a Gemini™ prep-RP-HPLC column from Phenomenex® Inc. (250 mm×21 mm, 5 mm, C₁₈) a specified linear gradient of solvent A, water (0.1% formic acid), and solvent B, acetonitrile (0.1% formic acid), with a flow rate of 10.6 mL/min. Chiral analytical Supercritical Fluid Chromatography (SFC) was performed on a Berger SFC Analytical Instrument equipped with a diode array UV detector using a Chiralpack® AD-H (250 mm×4.6 mm) or a Chiracel OD-H (250 mm×4.6 mm) column (Daicel Chemical Industries LTD, Osaka). MeOH was used as co-solvent to CO₂, flow rate was 3 g CO₂/min and back pressure was 150 bar.

### Example 1

### (2S)-Methionine methylsulfonium iodide

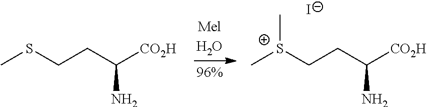

Methyl iodide (5.2 mL, 83.9 mmol) was added to a solution of L-methionine (5.0 g, 33.6 mmol) in water (50 mL) and the reaction mixture was stirred at 40° C. for 20 h. The mixture was then concentrated on a rotary evaporator and the resulting white solid was dissolved in a minimum volume of water (10 mL) with gentle heating, and treated with ethanol (70 mL) to give a white precipitate. The suspension was allowed to stand for 12 h allowing maximum precipitation. The precipitate was collected by filtration to give (2S)-methionine methylsulfonium iodide as a white solid (9.4 g, 96%). R_{f} 0.74 (50% ⁱPrOH/H₂O); mp: 163-165° C. (Lit.^{lib} 155-159° C.); [α]_{D}²⁰ +16.7 (c 1.0, H₂O) [Lit.^{lib} [α]_{D}²⁰ +17.0 (c 1.0, H₂O)]; ¹H NMR (400 MHz, D₂O) δ 2.37 (dt, 2H, J 8.0, 7.1, γ-CH₂), 2.95 (s, 6H, S(CH₃)₂), 3.41-3.54 (m, 2H, (β-CH₂) and 3.87 (t, 1H, J 6.4, α-CH); m/z (ESI) 164 (M⁺-I⁻, 92%) 142 (1) and 102 (6).

### Example 2

### (2R)-Methionine methylsulfonium iodide

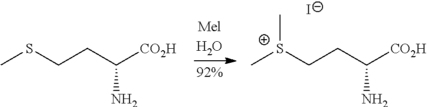

The reaction was carried out according to the above procedure using D-methionine (9.9 g, 66.6 mmol) to give (2R)-methionine methylsulfonium (17.5 g, 91%) as a white solid. [α]_{D}²⁰ −16.8 (c 1.0, H₂O); All other characterisation data was entirely consistent with that of the 2S-enantiomer.

### Example 3

### (S)-2-Amino-4-hydroxybutyric acid ((2S)-9) (L-homoserine)

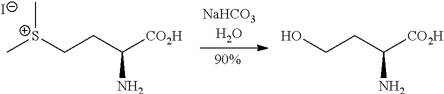

(2S)-Methionine methylsulfonium iodide (9.4 g, 32.3 mmol) was dissolved in water (30 mL) in a three-necked round-bottom flask and heated to reflux. Sodium bicarbonate (2.71 g, 32.3 mmol) was dissolved in water (40 mL), placed in a dropping funnel and added dropwise to the reaction mixture until the pH of the solution rose to 5.0 (monitored using a VWR symphony SB20 pH meter). The reaction was allowed to proceed until the pH dropped to 2.0 (˜15 min), whereupon base was added as previously described (addition over 6 h). The reaction mixture was then allowed to stir with heating at reflux for 12 h before being cooled, transferred to a 50 mL round-bottom flask and concentrated to give a clear oil. The oil was dissolved in water (5 mL) and ethanol (10 mL), and treated with acetone (150 mL) to produce a white precipitate that was collected by filtration. The precipitate was dissolved in water (20 mL) and freeze-dried, giving (S)-2-amino-4-hydroxybutyric acid (2S)-9 as a white solid (3.55 g, 92%). R_{f}=0.73 (40% EtOH/H₂O); mp: 175-177° C. (EtOH/Et₂O) (Lit.^{lia} mp: 182-183° C.); [α]_{D}²⁰ −8.5 (c 1.0, H₂O) [Lit.^{lia} [α]_{D}²⁰ −8.9 (c 2.0, H₂O)]; ¹H NMR (300 MHz, D₂O) [symbol] 1.86-1.93 (m, 1H, β-CHH), 1.99-2.05 (m, 1H, β-CHH), 3.63-3.67 (m, 2H, γ-CH₂) and 3.71 (dd, 1H, J 7.5, 4.8, α-CH); ¹³C NMR (75 MHz, D₂O) δ 32.9 (CH₂), 54.1 (CH), 59.4 (CH₂), 175.2 (C═O); m/z (ESI) 120 (MH⁺ , 100%) and 102 (1).

### Example 4

### (R)-2-Amino-4-hydroxybutyric acid (2R)-9 (D-homoserine)

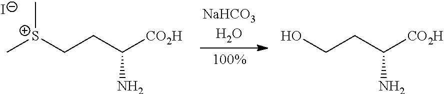

The reaction was carried out according to the above procedure using (2R)-methionine methylsulfonium (9.9 g, 66.6 mmol) to give (2R)-2-amino-4-hydroxybutyric acid (2R)-9 (7.1 g, 100%) as a white solid. [a]_{D}²⁰ 5.9 (c 1.0, H₂O) [L 8.8 (c 2.0, H₂O)]. All other characterization data was consistent with that of the 2S-enantiomer.

### Example 5

### (2S)-Benzyl-2-(N-(9-fluorenylmethoxycarbonyl)amino)-4-hydroxybutanoate [(2S)-10b]

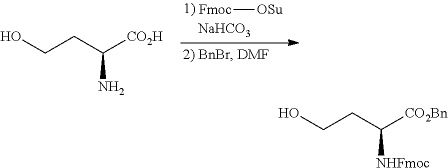

9-Fluorenylmethyl N-succinimidyl carbonate (Fmoc-OSu) (11.8 g, 35.0 mmol) was dissolved in acetone (150 mL) and added dropwise to a solution of (S)-2-amino-4-hydroxybutyric acid (2S)-9 (3.79 g, 31.9 mmol) and sodium hydrogen carbonate (5.35 g, 63.7 mmol) in water (150 mL) at 0° C., producing a white precipitate. The reaction mixture was allowed to stir for 18 h. Concentration of the solvent gave the sodium salt that was dissolved in water (100 mL) and freeze dried. The resulting anhydrous white solid was taken up in dry DMF (200 mL) and cooled to 0° C. Benzyl bromide (27.3 mL, 159.5 mmol) was added and the reaction mixture was allowed to stir for 3 h at 0° C. and then for 12 h at room temperature. The solvent was removed under high vacuum (without heating above 45° C.) and the resulting residue was taken up in water (100 mL) and extracted with ethyl acetate (3×100 mL). The combined organic layers were washed with water (2×50 mL), dried (MgSO₄) and concentrated to give a white solid. Purification was immediately carried out by column chromatography (70% diethyl ether/petroleum ether) to give (2S)-benzyl-2-(N-(9-fluorenylmethoxycarbonyl)amino)-4-hydroxybutanoate (2S)-10b (9.93 g, 72%) as a white solid. R_{f} 0.73 (50% EtOAc/Hexanes); mp: 105-107° C. (EtOAc/Et₂O) (Lit^{lvii} mp: 107-110° C.); [α]_{D}²⁰ −4.2 (c 1.0, CHCl₃) (from L-methionine), [α]_{D}²⁰ −8.9 (c 1.0, CHCl₃) (from L-homoserine purchased from Novabiochem) [Lit.^{lvii} [α]_{D} −6.2 (c 0.97, CHCl₃)]; Chiral SFC analysis (chromatogram provided hereafter, conditions: Chiracel® OD-H 250 mm×4.6 mm as column, at 35° C., with 20% MeOH as co-solvent, at a flow rate of 3 g CO₂/min and a pressure of 150 bar): (2S)-enantiomer t_{R} 17.73, (2R)-enantiomer t_{R} 12.13, 72% ee (from L-methionine), >99% ee (from purchased L-homoserine; chromatograms provided hereafter); ¹H NMR (400 MHz, CDCl₃) δ 1.72 (m, 1H, β-CHH), 2.18 (m, 1H, β-CHH), 2.99 (s, 1H, OH), 3.58-3.71 (m, 2H, γ-CH₂), 4.21 (t, 1H, J 6.7, Fmoc-CH), 4.45 (m, 2H, Fmoc-CH₂), 4.59 (m, 1H, α-CH), 5.16 (d, 1H, J=12.2, OCHHPh), 5.22 (d, 1H, J=12.2, OCHHPh), 5.86 (d, 1H, J 7.8, NH) and 7.31-7.78 (m, 13H, ar-H); ¹³C NMR (100 MHz, CDCl₃) δ 35.5 (CH₂), 47.2 (CH), 51.5 (CH), 58.4 (CH₂), 67.2 (CH₂), 67.4 (CH₂), 120.1 (ar-CH), 125.1 (ar-CH), 127.2 (ar-CH), 127.8 (ar-CH), 128.3 (ar-CH), 128.6 (ar-CH), 128.7 (ar-CH), 135.2 (ar-C), 141.4 (ar-C), 143.9 (ar-C), 156.8 (C═O) 172.5 (C═O); HRMS Calcd. for C₂₆H₂₆O₅N [M+H]⁺ 431.1806. found 432.1803.

### Example 6

### (2R)-Benzyl-2-(N-(9-fluorenylmethoxycarbonyl)amino)-4-hydroxybutanoate [(2R)-10b]

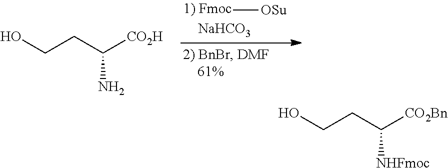

The reaction was carried out according to the above procedure using (2R)-2-amino-4-hydroxybutyric acid (2R)-9 (5.0 g, 42.0 mmol) giving (2R)-benzyl-2-(N-(9-fluorenylmethoxycarbonyl)amino)-4-hydroxybutanoate (2R)-10b (9.9 g, 61%) as a white solid. [α]_{D}²⁰ 4.1 (c 1.0, H₂O). All other characterization data was consistent with that of the 2S-enantiomer.

### Example 7

### (2S)-Methyl-2-(N-(9-fluorenylmethoxycarbonyl)amino)-4-hydroxybutanoate (10a)

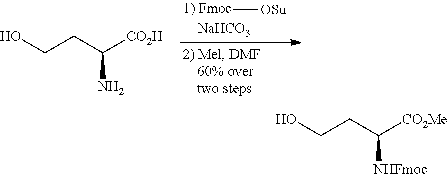

The reaction was carried out according to the above procedure using iodomethane (1.9 mL, 4.3 g, 30.3 mmol) and (2S)-2-amino-4-hydroxybutyric acid 9 (3.3 g, 27.7 mmol) giving (2S)-methyl-2-(N-(Fmoc)amino)-4-hydroxybutanoate (10a, 5.85 g, 60%) as a clear oil after purification by flash column chromatography (40% ethyl acetate I hexane). [α]²⁰_{D} −1.48 (c 0.54, CHCl₃); ¹H NMR (400 MHz, CDCl₃) δ 1.69-1.75 (m, 1H, n-CHH), 2.12-2.17 (m, 1H, β-CHH), 2.74-2.80 (m, 1H, OH), 3.56-3.64 (m, 1H, γ-CHH), 3.69-3.74 (m, 1H, γ-CHH), 3.77 (s, 3H, OMe), 4.22 (t, 1H, J 8.0 Hz, Fmoc-CH), 4.00-4.49 (m, 2H, Fmoc-CH₂), 4.53-4.56 (m, 1H, α-CH), 5.72 (d, 1H, J 8.0, NH), 7.30 (t, 2H, J 8.0, 2×ar-H), 7.39 (t, 2H, J 8.0, 2×ar-H), 7.60 (d, 2H, J 8.0, 2×ar-H), 7.76 (d, 2H, J 8.0, 2×ar-H); ¹³C NMR (100 MHz, CDCl₃) δ 35.5 (CH₂), 47.1 (CH), 51.1 (CH₃), 52.6 (CH), 58.3 (CH₂), 67.1 (CH₂), 120.0 (ar-H), 125.0 (ar-H), 127.1 (ar-H), 127.7 (ar-H), 141.3 (ar-H), 143.6 (ar-H), 156.7 (C═O), 173.0 (C═O); υₘₐₓ/cm⁻¹ (NaCl): 3316 (OH), 2947 (CH), 1690 (CO), 1537 (C═C), 1450, 1272, 1213, 1051 and 739; HRMS Calcd. For C₂₀H₂₂NO₅ [M+H]⁺ 356.1492. found 356.1481.

### Example 8

### (2S,4S)- and (2R,4S)-Benzyl-2-oxo-3-(9-fluorenylmethoxycarbonyl)-1,2,3-oxathiazinane-4-carboxylate [(2S)-11b and (2R)-11b]

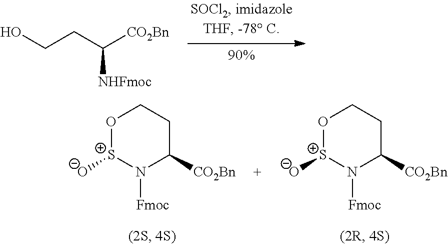

Thionyl chloride (0.51 mL, 6.96 mmol) was added drop-wise to a solution of imidazole (1.89 g, 27.8 mmol) in THF (150 mL) at −78° C., producing a white precipitate. This mixture was allowed to stir for 0.5 h, filtered through a plug of Celite™ and the precipitate was washed with THF (50 mL). The combined filtrate and washings were cooled to 0° C., treated drop-wise over 0.5 h with a solution of (2S)-benzyl-2-(N-(9-fluorenylmethoxycarbonyl)amino)-4-hydroxybutanoate (2S)-10b (1.0 g, 2.32 mmol) in THF (100 mL), stirred for 4 h at 0° C. and then for 1 h at room temperature. The mixture was concentrated and immediately purified by flash column chromatography (40% EtOAc/hexanes) to give a 2:1 mixture of (2S,4S)- and (2R,4S)-benzyl-2-oxo-3-(9-fluorenylmethoxycarbonyl)-1,2,3-oxathiazinane-4-carboxylate [(2S,4S)-11b and (2R,4S)-11b (1.0 g, 90%)]. First to elute was (2S,4S)-benzyl-2-oxo-3-(9-fluorenylmethoxycarbonyl)-1,2,3-oxathiazinane-4-carboxylate [(2S,4S)-11b]: R_{f} 0.8 (50% EtOAc/Hexanes); [α]_{D}²⁰ 51.4 (c 1.0, CHCl₃); ¹H NMR (400 MHz, CDCl₃) [symbol] 2.36-2.65 (m, 2H, β-H₂), 3.94-3.99 (m, 1H, α-CH), 4.18 (m, 1H, Fmoc-CH), 4.33 (dd, 1H, J 7.6, 10.4, Fmoc-CHH), 4.49-4.55 (m, 1H, Fmoc-CHH), 4.94-5.01 (m, 2H, γ-CH₂), 5.17 (d, 1H, J 12.2, OCHHPh), 5.23 (d, 1H, J 12.2, OCHHPh) and 7.21-7.79 (m, 13H, ar-H); ¹³C NMR (100 MHz, CDCl₃) [symbol] 24.1 (CH₂), 46.9 (CH), 55.3 (CH₂), 68.0 (CH₂), 69.5 (CH₂), 120.2 (ar-CH), 125.2 (ar-CH), 127.4 (ar-CH), 128.1 (ar-CH), 128.3 (ar-CH), 128.4 (ar-CH), 128.6 (ar-CH), 128.8 (ar-CH) 135.2 (ar-C), 141.4 (ar-C), 141.5 (ar-C), 143.6 (0=0) and 169.6 (0=0); υₘₐₓ/cm⁻¹ (NaCl): 2953 (CH), 1735 (CO), 1450 (C═C), 1289, 1198 and 739; HRMS Calcd. for C₂₆H₂₃O₆NSNa [M+Na]⁺ 500.1138. found 500.1145. Second to elute was (2R,4S)-benzyl-2-oxo-3-(9-fluorenylmethoxycarbonyl)-1,2,3-oxathiazinane-4-carboxylate (2R,4S)-11b: R_{f} 0.76 (50% EtOAc/Hexanes); [α]_{D}²⁰ −9.7 (c 0.5, CHCl₃); ¹H NMR (400 MHz, CDCl₃) [symbol] 2.69-2.79 (m, 2H, β-CH₂), 4.02-4.59 (m, 4H, α-CH, γ-CH₂ and Fmoc-CH), 4.93-5.01 (m, 2H, Fmoc-CH₂), 5.18-5.24 (m, 2H, OCH₂Ph) and 7.23-7.80 (m, 13H, ar-H); ¹³C NMR (100 MHz, CDCl₃) [symbol] 25.6 (CH₂), 47.6 (CH), 52.1 (CH), 57.9 (CH₂), 68.6 (CH₂), 120.9 (ar-CH), 126.0 (ar-CH), 128.1 (ar-CH), 128.8 (ar-CH), 128.9 (ar-CH), 129.4 (ar-CH), 129.5 (ar-CH), 135.9 (ar-C), 142.1 (ar-C), 144.0 (ar-C) and 170.3 (0=0); υₘₐₓ/cm⁻¹ (NaCl): 2954 (CH), 1729 (CO), 1450 (C═C), 1305, 1183, 1095 and 967; HRMS Calcd. for C₂₆H₂₃O₆NSNa [M+Na]⁺ 500.1138. found 500.1144.

### Example 9

### (2R,4R)- and (2S,4R)-benzyl-2-oxo-3-(9-fluorenylmethoxycarbonyl)-1,2,3-oxathiazinane-4-carboxylate [(2R,4R)-11b and (2S,4R)-11b]

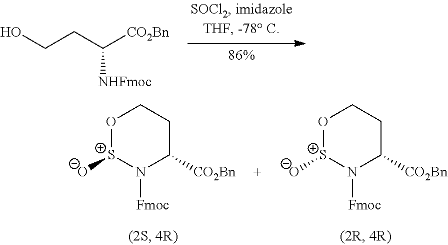

The reaction was carried out according to the above procedure using (2R)-benzyl-2-(N-(9-fluorenylmethoxycarbonyl)amino)-4-hydroxybutanoate [(2R)-10b, 2.0 g, 4.64 mmol] to give a 2:1 mixture of (2R,4R)- and (2S,4R)-benzyl-2-oxo-3-(9-fluorenylmethoxycarbonyl)-1,2,3-oxathiazinane-4-carboxylates [(2R,4R)- and (2S,4R)-11b, 1.91 g, 86%) as a clear oil. First to elute was (2R,4R)-benzyl-2-oxo-3-(9-fluorenylmethoxycarbonyl)-1,2,3-oxathiazinane-4-carboxylate [(2R,4R)-11b]: [α]_{D}²⁰ −49.1 (c 1.0, CHCl₃). All other characterization data were consistent with that of its (2S,4S)-enantiomer. Second to elute was (2S,4R)-benzyl-2-oxo-3-(9-fluorenylmethoxycarbonyl)-1,2,3-oxathiazinane-4-carboxylate [(2S,4R)-11b]: [α]_{D}²⁰ 8.4 (c 0.5, CHCl₃). All other characterization data was consistent with that of its (2R,4S)-enantiomer.

### Example 10

### (2S,4S)- and (2R,4S)-Methyl 2-oxo-3-Fmoc-1,2,3-oxathiazinane-4-carboxylate (2S,4S)- and (2R,4S)-11a

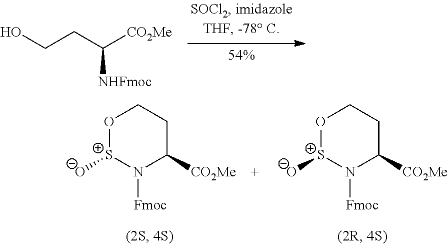

The reaction was carried out according to the above procedure using (2S)-methyl-2-(N-(9-fluorenylmethoxycarbonyl)amino)-4-hydroxybutanoate [(2S)-10a, 0.5 g, 1.41 mmol] to give a 4:1 mixture of (2S,4S)- and (2R,4S)-methyl 2-oxo-3-Fmoc-1,2,3-oxathiazinane-4-carboxylates [(2S,4S)- and (2R,4S)-11a, 0.31 g, 54%]. First to elute was (2S,4S)-methyl 2-oxo-3-Fmoc-1,2,3-oxathiazinane-4-carboxylate [(2S,4S)-11a]; R_{f} 0.22 (10% EtOAc/hexane); [α]²⁰_{D} 32.5 (c 0.44, CHCl₃) ¹H NMR (400 MHz, CDCl₃) δ 2.30-2.40 (m, 1H, β-CHH), 2.35-2.47 (m, 1H, β-CHH), 3.78 (s, 3H, OMe), 3.95-3.97 (m, 1H, α-CH), 4.32-4.35 (m, 1H, Fmoc-CH), 4.40-4.49 (m, 1H, Fmoc-CHH), 4.62-4.66 (m, 1H, Fmoc-CHH), 4.90-4.97 (m, 2H, γ-CH₂), 7.32 (t, 2H, J 8.0, 2×ar-H), 7.44 (t, 2H, J 8.0, 2×ar-H), 7.59 (t, 2H, J 8.0, 2×ar-H), 7.78 (d, 2H, J 8.0, 2×ar-H); ¹³C NMR (100 MHz, CDCl₃) δ 24.7 (CH₂), 46.7 (CH), 50.9 (CH₃), 53.1 (CH₂), 57.0 (CH), 69.2 (CH₂), 120.0 (ar-CH), 125.1 (ar-CH), 127.2 (ar-CH), 127.9 (ar-CH), 141.3 (ar-C), 143.1 (ar-C), 170.1 (0=0); υₘₐₓ/cm⁻¹ (NaCl): 2952 (CH), 1735 (CO), 1450 (C═C), 1386, 1290, 1212 and 739; HRMS Calcd. For C₂₀H₁₉NO₆SNa [M+Na]⁺ 424.0825. found 424.0818. Second to elute was (2R,4S)-methyl 2-oxo-3-Fmoc-1,2,3-oxathiazinane-4-carboxylate [(2R,4S)-11a]: R_{f} 0.19 (10% EtOAc/hexane); [α]²⁰_{D}: hexane −28.5 (c=0.4, CHCl₃); ¹H NMR (400 MHz, CDCl₃) δ 2.65-2.78 (m, 2H, β-CH₂), 3.79 (s, 3H, OMe), 4.11-4.18 (m, 2H, α-CH and g-CHH), 4.32 (t, 1H, J 8.0, Fmoc-CH), 4.53 (d, 2H, J 8.0, Fmoc-CH₂), 4.57-4.64 (m, 1H, γ-CHH), 7.33 (t, 2H, J 8.0, 2×ar-CH), 7.42 (t, 2H, J 8.0, 2×ar-CH), 7.63 (m, 2H, 2×ar-CH), 7.87 (d, 2H, J 8.0, 2×ar-CH); ¹³C NMR (100 MHz, CDCl₃) [symbol] 23.9 (CH₂), 46.7 (CH), 49.7 (CH₃), 52.6 (CH₂), 55.2 (CH), 69.3 (CH₂), 120.7 (ar-CH), 125.0 (ar-CH), 127.2 (ar-CH), 128.0 (ar-CH), 141.1 (ar-C), 141.2 (ar-C), 143.2 (ar-C), 143.4 (C═O), 170.2 (C═O); υₘₐₓ/cm⁻¹ (NaCl): 2954 (CH), 1730 (CO), 1450 (C═C), 1389, 1306, 1184 and 758; HRMS Calcd. For C₂₀H₁₉NO₆SNa [M+Na]⁺ 424.0825. found 424.0830.

### Example 11

### (4S)-Benzyl-2,2-dioxo-3-(9-fluorenylmethoxycarbonyl)-1,2,3-oxathiazinane-4-carboxylate ((4S)-8)

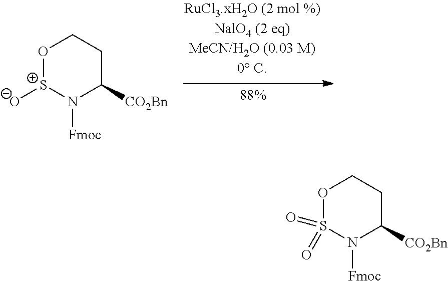

A solution of sodium periodate (0.9 g, 4.2 mmol) in water (70 mL) was added to a solution of (2SR,4S)-benzyl-2-oxo-3-(9-fluorenylmethoxycarbonyl)-1,2,3-oxathiazinane-4-carboxylate [(2SR,4S)-11b, 1.0 g, 2.09 mmol] in acetonitrile (70 mL) at 0° C. Ruthenium(III) chloride hydrate (9 mg, 0.04 mmol) was added to the biphasic solution, which was stirred vigorously for 5 h at 0° C. The reaction mixture was diluted with diethyl ether (50 mL) and the two layers were separated. The aqueous layer was extracted with diethyl ether (3×50 mL) and the combined organic layers were then washed with saturated sodium bicarbonate solution (2×50 mL) and brine (50 mL), dried (MgSO₄) and concentrated in vacuo to give (4S)-benzyl-2,2-dioxo-3-(9-fluorenylmethoxycarbonyl)-1,2,3-oxathiazinane-4-carboxylate [(4S)-8, 0.91 g, 88%] as a white solid. R_{f} 0.70 (50% EtOAc/Hexanes); [α]_{D}²⁰ −37.4 (c 1.0, CHCl₃, from L-methionine) [α]_{D}²⁰ −40.5 (c 1.0, CHCl₃, from purchased L-homoserine); Chiral SFC analysis (chromatograms provided hereafter, conditions: (4R)-enantiomer t_{R} 8.82, (4S)-enantiomer t_{R} 13.55, 80% ee (from L-Methionine), 99% (from purchased L-homoserine); ¹H NMR (400 MHz, CDCl₃) δ 2.45-2.57 (m, 1H, δ-CHH), 2.70 (ddt, 1H, J 14.5, 5.1, 2.5, β-CHH), 4.25 (t, 1H, J 7.5, Fmoc-CH), 4.38-4.49 (m, 2H, Fmoc-CH₂), 4.66-4.84 (m, 2H, γ-CH₂), 5.20 (d, 1H, J 12.2, OCHHPh), 5.25 (d, 1H, J 12.2, OCHHPh), 5.33 (dd, 1H, J 5.1, 2.5, α-CH), 7.24-7.44 (m, 9H, ar-CH) and 7.66-7.79 (m, 4H, ar-CH); ¹³C NMR (100 MHz, CDCl₃) [symbol] 24.7 (CH₂), 46.7 (CH), 58.0 (CH), 68.6 (CH₂), 71.0 (CH₂), 71.2 (CH₂), 120.1 (ar-CH), 125.6 (ar-CH), 127.5 (ar-CH), 128.1 (ar-CH), 128.4 (ar-CH), 128.8 (ar-CH), 134.8 (ar-CH), 141.5 (ar-C), 143.2 (ar-C), 143.4 (C), 152.4 (C═O), 168.3 (C═O); υₘₐₓ/cm⁻¹ (NaCl): 2954 (CH), 1741 (CO), 1451 (C═C), 1396, 1179 and 972; HRMS Calcd. for C₂₆H₂₃O₇NSNa [M+Na]⁺ 516.1076. found 516.1087.

### Example 12

### (4R)-Benzyl-2,2-dioxo-3-(9-fluorenylmethoxycarbonyl)-1,2,3-oxathiazinane-4-carboxylate (4R)-8

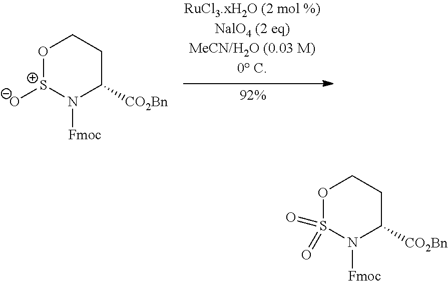

The reaction was carried out according to the above procedure using (2SR,4R)-benzyl-2-oxo-3-(9-fluorenylmethoxycarbonyl)-1,2,3-oxathiazinane-4-carboxylate [(2SR,4R)-11b, 0.9 g, 1.89 mmol] to give (4R)-benzyl-2,2-dioxo-3-(9-fluorenylmethoxycarbonyl)-1,2,3-oxathiazinane-4-carboxylate [(4R)-8, 0.86 g, 92%] as a white solid: [α]_{D}²⁰ 35.5 (c 1.0, CHCl₃). Chiral SFC analysis (chromatogram provided hereafter, conditions: Chiracel® OD-H 250 mm×4.6 mm as column, at 35° C., with 20% MeOH as co-solvent, at a flow rate of 3 g CO₂/min and a pressure of 150 bar): (4R)-enantiomer t_{R} 8.82, (4S)-enantiomer t_{R} 13.55, 90% ee (from D-methionine). All other characterization data was consistent with that of the 4S-enantiomer.

### Example 13

### (4S)-Methyl-2,2-dioxo-3-Fmoc-1,2,3-oxathiazinane-4-carboxylate [(4S)-7]

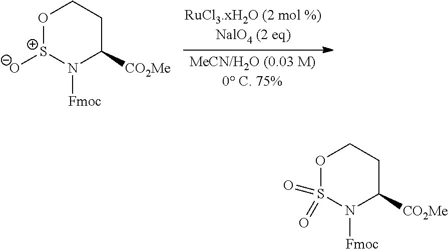

The reaction was carried out according to the above procedure using (2SR,4S)-methyl 2-oxo-3-Fmoc-1,2,3-oxathiazinane-4-carboxylate [(2SR,4S)-11a, 0.25 g, 0.62 mmol] to give (4S)-methyl-2,2-dioxo,3-Fmoc-1,2,3-oxathiazinane-4-carboxylate [(4S)-7, 0.195 g, 75%] as a clear oil: [α]²⁰_{D} −1.29 (c=0.54, CHCl₃); ¹H NMR (400 MHz, CDCl₃) δ 2.05-2.54 (m, 1H, β-CHH), 2.68-2.73 (m, 1H, δ-CHH), 3.82 (s, 3H, OMe), 4.37 (t, 1H, J 4.0, Fmoc-CH), 4.50 (dd, 1H, J 10.4 and 8.0, Fmoc-CHH), 4.61 (dd, 1H, J 10.4 and 8.0, Fmoc-CHH), 4.72 (m, 1H, β-CHH), 4.81 (m, 1H, (3-CHH), 5.30 (dd, 1H, J 5.8 and 2.6, α-CH), 7.34 (td, 2H, J 7.5 and 0.9, 2×ar-H), 7.42 (t, 2H, J 7.5, 2×ar-H), 7.73-7.78 (m, 4H, 4×ar-H); ¹³C NMR (100 MHz, CDCl₃) δ 24.4 (CH₂), 46.5 (CH), 53.4 (CH₃), 57.5 (CH₂), 70.8 (CH), 71.0 (CH₂), 120.0 (ar-CH), 125.4 (ar-CH), 127.3 (ar-CH), 125.4 (ar-CH), 127.3 (ar-CH), 128.0 (ar-CH), 141.3 (C), 143.0 (C), 152.0 (C═O), 168.7 (C═O); υₘₐₓ/cm⁻¹ (NaCl): 2917 (CH), 1742 (CO), 1450 (C═C), 1393, 1299 and 1179; HRMS Calcd. For C₂₀H₁₉NO₇SNa [M+Na]⁺ 440.0774. found 440.0769.

### Example 14

### L-(2S)-β-Methyl-N-(9-fluorenylmethoxycarbonyl)aspartate ((2S)-15)

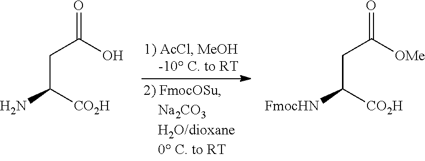

HCl-L-Asp(OMe)-OH was synthesized according to the procedure of Gmeiner et al.^{liv} Acetyl chloride (3.75 mL, 52.6 mmol) was added to dry MeOH (13 mL) at 5° C. The solution was stirred for 30 min in an ice bath and then added to a cooled (ice bath) suspension of L-aspartic acid (5 g, 37.6 mmol) in dry MeOH (13 mL). L-Aspartic acid was dissolved completely within a few minutes and the solution was stirred at 8-10° C. for 3 h, at which point the ice bath was removed and stirring was continued for 15 h. The reaction mixture was then poured into ice-cold Et₂O (70 mL) and upon cooling and stirring, a white solid precipitated, which was filtered immediately and washed with ice-cold diethyl ether. The filtrate and washes were combined and evaporated to give residue from which a second crop was obtained by solubilization in MeOH (6 mL) and addition of cold Et₂O (20 mL). The white solid (5.5 g) was then dried under high vacuum for two hours and consisted in a 85/15 mixture of HCl-L-Asp(OMe)-OH and HCl-L-Asp(OMe)-OMe (as judged by ¹H NMR) which was used as such for the next step. The mixture was dissolved in water (250 mL) and treated with sodium carbonate (9.9 g, 93.9 mmol). After complete dissolution of the solids, the solution was cooled to 5° C. in an ice bath and treated dropwise over 1 h with a solution of 9-fluorenylmethyl N-succinimidyl carbonate (Fmoc-OSu, 16.5 g, 48.8 mmol) in dioxane (500 mL). The reaction mixture was allowed to warm slowly to room temperature and stirring was continued for 15 h. The volatiles were then removed by rotary evaporation to furnish an aqueous solution, which was washed with EtOAc (3×200 mL), acidified with 12 N HCl to pH=2 and extracted with EtOAc (3×200 mL). The later organic layers were combined, dried (MgSO₄) and concentrated to give a colorless oil which yielded a white solid after trituration with hexanes for 2 h. The solid was filtered and dryed under vacuum to provide L-(2S)-γ-methyl-N-(9-fluorenylmethoxycarbony)aspartate (15, 11.4 g, 82% over two steps); mp: 122-125° C., [α]²³_{D} 14.6 (c 1.05, CHCl₃); ¹H NMR (CDCl₃, 400 MHz) δ 2.91 (dd, 1H, β-CHH, J=17.4, 4.5), 3.11 (dd, 1H, β-CHH, J=17.4, 4.5), 3.73 (s, 3H, CH₃), 4.23 (t, 1H, Fmoc-CH, J=7.1), 4.38 (dd, 1H, Fmoc-CHH, J=10.5, 7.3), 4.44 (dd, 1H, Fmoc-CHH, J=10.5, 7.3), 4.71 (dt, 1H, β-CH, J=8.5, 4.5), 5.92 (d, 1H, NH, J=8.5), 7.31 (dt, 2H, 2×ar-H, J=7.4, 1.1), 7.40 (t, 2H, 2×ar-H, J=7.4), 7.60 (d, 2H, 2×ar-H, J=7.1), 7.76 (d, 2H, 2×ar-H, J=7.5), 9.72 (s, 1H, CO₂H); ¹³C NMR (75 MHz, CDCl₃) δ 37.1 (CH₂), 47.9 (CH), 51.0 (CH), 53.1 (CH₃), 67.3 (CH₂), 120.9 (ar-CH), 126.0 (ar-CH), 128.0 (ar-CH), 128.6 (ar-CH), 142.1 (ar-C), 144.5 (ar-C), 157.0 (C═O), 172.5 (C═O), 176.2 (C═O); HRMS calcd. for C₂₀H₁₉NO₆Na [M+Na]⁺ : 392.1104. found: 392.10984; υₘₐₓ/cm⁻¹ (KBr): 3376 (N—H), 3064 (O—H), 2948 (C—H), 1741, 1738, 1695 (C═O), 1533 (N—H), 1449 (C—H alkyl), 1211, 1168 and 739.

### Example 15

### (S)-Methyl-3-[N-(9-fluorenylmethoxycarbonyl)amino]-4-hydroxybutanoate [(S)-16]

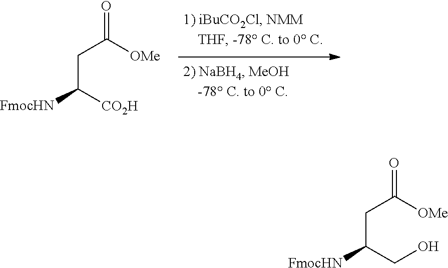

A solution of L-(2S)-γ-methyl-N-(9-fluorenylmethoxycarbonyl)aspartate (15, 10 g, 28.14 mmol) in dry THF (200 mL) was cooled to −78° C., under an argon atmosphere, and treated with N-methylmorpholine (3.56 mL, 32.36 mmol) followed by isobutyl chloroformate (4.26 mL, 32.36 mmol). After stirring for 10 minutes, the reaction mixture was allowed to slowly warm to 0° C. over 40 min and cooled to −78° C. and treated with a portion of sodium borohydride (3.19 g, 84.42 mmol), followed by drop-wise addition of methanol (100 mL) over 30 min. The mixture was then stirred at −78° C. for 30 min, allowed to warm to −20° C. over 1 h, at which point the reaction was complete as judged by consumption of starting material by TLC (on an hydrolyzed aliquot, 80% EtOAc/hexanes, bromocresol green revelation, R_{f} of 15=0.11) The reaction was quenched with saturated NH₄Cl (100 mL). The volatiles were evaporated under reduced pressure and the resulting aqueous phase was extracted with ethyl acetate (3×100 mL). The combined organic layers were washed with water (100 mL) and brine (100 mL), dried over anhydrous magnesium sulfate, filtered and concentrated. The residue was dissolved in the minimum amount of hot CHCl₃, and the expected (S)-methyl 3-[N-(9-fluorenylmethoxycarbonyl)amino]-4-hydroxybutanoate [(2S)-16] was purified by flash chromatography (50% EtOAc in hexanes) to give a white solid (7.54 g, 75%): R_{f} 0.24 (50% EtOAc/hexanes); mp 133-135° C.; [α]²³_{D} −10.7 (c 2.0, MeOH); ¹H NMR (CDCl₃, 700 MHz) δ 2.50 (t, 1H, OH, J=5.5), 2.69 (d, 2H, CH₂—CO₂Et, J=5.8), 3.72 (s, 3H, CH₃), 3.75 (dd, 2H, CH₂—OH, J=3.8, 5.0), 4.08 (m, 1H, CH—NH), 4.23 (t, 1H, Fmoc-CH, J=6.8), 4.42 (m, 2H, Fmoc-CH₂), 5.54 (d, 1H, NH, J=7.85), 7.34 (t, 2H, 2×ar-H, J=7.4), 7.42 (t, 2H, 2×ar-H, J=7.4), 7.60 (d, 2H, 2×ar-H, J=7.4), 7.78 (d, 2H, 2×ar-H, J=7.4); ¹³C NMR (175 MHz, CDCl₃) δ 35.7 (CH₂), 47.2 (CH), 49.7 (CH), 52.0 (CH₃), 63.3 (CH₂), 66.9 (CH₂), 120.0 (ar-CH), 125.1 (ar-CH), 127.1 (ar-CH), 127.8 (ar-CH), 141.3 (ar-C), 143.8 (ar-C), 156.3 (C═O), 172.3 (C═O); HRMS calcd. for C₂₀H₂₁NO₅Na [M+Na]⁺ : 378.1312. found: 378.1309; υₘₐₓ/cm⁻¹ (KBr): 3320 (O—H), 2957 (C—H), 1731, 1693 (C═O), 1547 (N—H), 1443 (C—H alkyl), 1271 (C—O ester), 1022 (C—O alcohol) and 738.

The enantiomeric (R)-methyl-3-[N-(9-fluorenylmethoxycarbonyl)amino]-4-hydroxybutanoate [(2R)-16] was prepared from D-aspartic acid according to the same route used to prepare (2S)-16: [∝]²³_{D} 11.4 (c 2.0, MeOH). All other characterization data was consistent with that of the (S)-enantiomer. Chiral SFC analysis (chromatograms provided hereafter, conditions: Chiralpak® AD-H 250 mm×4.6 mm as column, at 40° C., with 30% MeOH as co-solvent, at a flow rate of 3 g CO₂/min and a pressure of 149 bar): (2S)-enantiomer t_{R} 5.4, 99.3% ee; (2R)-enantiomer t_{R} 15.8, 98.7% ee.

### Example 16

### (4S)-Methyl-2-[3-(9-fluorenylmethoxycarbonyl)-2,2-dioxo-1,2,3-oxathiazolidin-4-yl]acetate [(4S)-17]

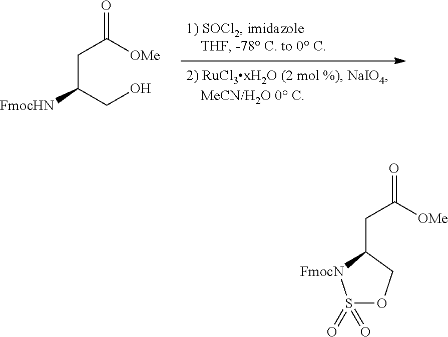

Thionyl chloride (0.62 mL, 8.44 mmol) was added dropwise to a solution of imidazole (2.30 g, 33.77 mmol) in THF (125 mL) at −78° C., producing a white precipitate. After stirring for 0.5 h at −78° C., the mixture was treated with a solution of (S)-methyl 3-[N-(9-fluorenylmethoxycarbonyl)amino]-4-hydroxybutanoate [(2S)-16, 1 g, 2.81 mmol) in THF. The bath was removed. The mixture was allowed to warm to room temperature with stirring for two hours and filtered through Celite™. The filtrate was concentrated to a residue which was partitioned between water (20 mL) and DCM (50 mL). The aqueous phase was extracted with DCM (50 mL), and the combined organic layers were washed with 10% HCl (20 mL) and brine (20 mL), dried (MgSO₄) and evaporated in vacuo. The residue was dissolved in acetonitrile (21 mL), cooled to 0° C., and treated with portions of solid ruthenium (III) chloride hydrate (12 mg, 0.06 mmol) and sodium periodate (0.903 g, 4.22 mmol), followed by water (12 mL), drop-wise over 10 minutes. The mixture was stirred at 0° C. for 2 hours and partitioned between ethyl acetate (100 mL) and water (20 mL). The aqueous phase was extracted with ethyl acetate (2×50 mL) and the combined organic layers were washed with saturated sodium bicarbonate (50 mL) and brine (50 mL). The solution was dried over anhydrous magnesium sulfate and concentrated in vacuo to afford an orange oil, which was purified by flash chromatography (EtOAc/hexane 6/4). Evaportation of the collected fractions afforded a colorless oil (0.8 g, 85%). Overnight crystallization at 4° C. from diethyl ether (5 mL) and filtration gave (4S)-methyl-2-[3-(9-fluorenylmethoxycarbonyl)-2,2-dioxo-1,2,3-oxathiazolidin-4-yl]acetate [(4S)-17, 0.79 g, 76%)] as white crystals: R_{f} 0.44 (50% EtOAc/Hexanes); mp 138-140° C.; [α]²³_{D} 33.2 (c 1.04 in CHCl₃); ¹H NMR (CDCl₃, 700 MHz) δ 2.83 (m, 2H, CH₂—CO₂Me), 3.76 (s, 3H, CH₃), 4.35 (t, 1H, Fmoc-CH, J=6.9), 4.58 (m, 2H, Fmoc-CH₂), 4.62 (m, 1H, CH—N), 4.84 (m, 2H, CH₂—O—SO₂), 7.37 (tt, 2H, 2×ar-H, J=7.5, 1.4), 7.43 (t, 1H, ar-H, J=7.4), 7.44 (t, 1H, ar-H, J=7.4), 7.71 (t, 2H, 2×ar-H, J=7.4), 7.78 (d, 1H, ar-H, J=7.4), 7.79 (d, 1H, ar-H, J=7.4); ¹³C NMR (175 MHz, CDCl₃) δ 35.1 (CH₂), 46.5 (CH), 52.3 (CH), 54.3 (CH₃), 70.3 (2 CH₂), 120.1 (ar-CH), 125.2 (ar-CH), 127.4 (ar-CH), 128.1 (ar-CH), 141.3 (ar-C), 142.8 (ar-C), 149.5 (C═O), 170.1 (C═O); HRMS calcd. for C₂₀H₁₃NO₇Na [M+Na]⁺ : 440.0774. found: 440.0770; υₘₐₓ/cm⁻¹ (KBr): 2949 (C—H), 1744, 1698 (C═O), 1443 (C—H alkyl), 1393,1384 (S═O), 1318, 1291 (C—O ester), 1197 (S—O), 1182 (S—N) and 824.

Enantiomeric (4R)-methyl-2-[3-(9-fluorenylmethoxycarbonyl)-2,2-dioxo-1,2,3-oxathiazolidin-4-yl]acetate [(4R)-17] was prepared from (2R)-16 according to the same route used to prepare (4S)-17: [α]²³_{D} −35.0 (c 1.0 in CHCl₃). All other characterization data was consistent with that of the (4S)-enantiomer. Chiral SFC analysis (chromatograms provided hereafter, conditions: Chiracel® OD-H 250 mm×4.6 mm as column, at 40° C., with 20% MeOH as co-solvent, at a flow rate of 3 g CO₂/min and a pressure of 150 bar): (S)-enantiomer t_{R} 5.97, 95.8% ee; (R)-enantiomer t_{R} 6.68, 97.9% ee.

### Example 17

### (S)-Benzyl-4-[(S)-3-(1H-indol-3-yl)-1-methoxy-1-oxopropan-2-ylamino]-2-[N-(9-fluorenylmethoxycarbonyl)amino]butanoate (12)

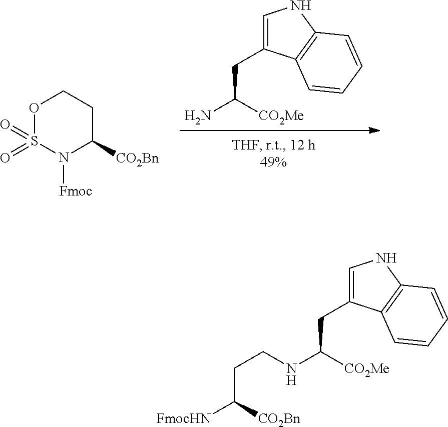

Tryptophan methyl ester hydrochloride (70 mg, 0.27 mmol) was dissolved in water (5 mL), saturated with potassium carbonate and extracted with chloroform (5×10 mL). The combined organic layers were dried (MgSO₄) and concentrated in vacuo to give the free amino ester, which was subsequently dissolved in THF (1 mL) and added to a solution of (4S)-benzyl-2,2-dioxo-3-(9-fluorenylmethoxycarbonyl)-1,2,3-oxathiazinane-4-carboxylate [(4S)-8, 45 mg, 0.09 mmol] in THF (1 mL). The reaction mixture was stirred at room temperature for 12 h, and treated with 1M mono-potassium phosphate (4 mL). The layers were separated. The aqueous layer was extracted with EtOAc (4×10 mL). The combined organic layers were dried (MgSO₄) and concentrated to a solid that was purified by flash column chromatography^{lvii} (30% EtOAc in hexanes). Evaporation of the collected fractions gave (S)-benzyl 4-[(S)-3-(1H-indol-3-yl)-1-methoxy-1-oxopropan-2-ylamino]-2-[N-(9-fluorenylmethoxycarbonyl)amino]butanoate (12, 28 mg, 49%) as a white solid: R_{f} 0.29 (50% EtOAc/Hexanes): [∝_{D}²⁰ −7.3 (c 1.0, CHCl₃); ¹H NMR (400 MHz, CDCl₃) δ 1.80-1.97 (m, 2H, 2′-H₂), 2.51 (m, 1H, 1′-HH), 2.72 (m, 1H, 1′-HH), 3.06 (dd, 1H, J 14.4, 5.5, 3-HH), 3.15 (dd, 1H, J 14.4, 7.1, 3-HH), 3.56 (t, 1H, J 6.1, 2-H), 3.63 (s, 3H, OMe), 4.23 (t, 1H, J 6.5, Fmoc-CH), 4.34-4.49 (m, 3H, 2′-H and Fmoc-CH₂), 5.11 (d, 1H, J 12.2, OCHHPh), 5.17 (d, 1H, J 12.2, OCHHPh), 6.55 (br d, 1H, J 7.4, 3′—NH), 6.96-7.76 (m, 19H, ar-H) and 7.94 (s, 1H, Trp-H); ¹³C NMR (100 MHz, CDCl₃) δ 28.9 (CH₂), 30.7 (CH₂), 44.1 (CH₂), 47.1 (CH), 51.8 (CH), 53.1 (CH), 61.3 (CH₃), 66.6 (CH₂), 66.9 (CH₂), 111.1 (ar-CH), 118.4 (ar-CH), 119.3 (ar-CH), 119.8 (ar-CH), 121.9 (ar-CH), 123.0 (ar-CH), 124.9 (ar-CH), 125.0 (ar-CH), 126.9 (ar-CH), 127.1 (ar-C), 127.6 (ar-CH), 128.1 (ar-CH), 128.4 (ar-CH), 135.3 (ar-C), 136.0 (ar-C), 141.1 (ar-C), 143.7 (ar-C), 143.9 (ar-C), 156.1 (ar-C), 172.0 (C═O) and 174.3 (C═O); υₘₐₓ/cm⁻¹ (NaCl): 3338 (NH), 2952 (CH), 1723 (C═O), 1451 (C═C), 1070, 959 and 741; HRMS Calcd. for C₃₈H₃₈O₆N₃ [M+H]⁺ 632.2755. found 632.2755.

### Example 18

### (S)-Methyl-2-{(S)-3-[N-(9-fluorenylmethoxycarbonyl)amino]-2-oxopyrrolidin-1-yl}-3-(1H-indol-3-yl)propanoate (13)

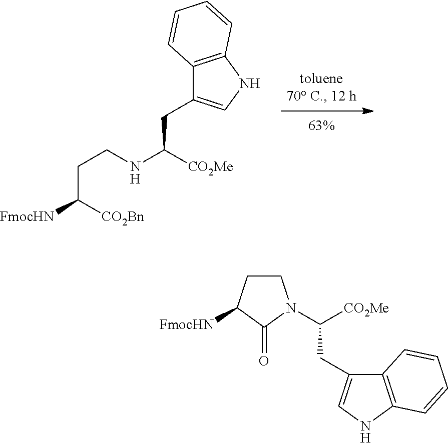

(S)-Benzyl 4-[(S)-3-(1H-indol-3-yl)-1-methoxy-1-oxopropan-2-ylamino]-2-[N-(9-fluorenyl-methoxycarbonyl)amino]butanoate (12, 9 mg, 0.02 mmol) was dissolved in toluene (3 mL) and stirred at 70° C. for 12 h, when LC/MS analysis indicated complete consumption of starting material: analytical RP-HPLC using 20-80% MeCN (0.1% formic acid) in water (0.1% formic acid) over a 4 min gradient; amine 12, t_{R} 5.35; lactam 13, t_{R} 7.69. The reaction mixture was concentrated on a rotary evaporator and purified by column chromatography (30% EtOAc/hexanes) to give (S)-methyl 2-{(S)-3-[N-(9-fluorenylmethoxycarbonyl)amino]-2-oxopyrrolidin-1-yl}-3-(1H-indol-3-yl)propanoate 13 as a white foam (5 mg, 63%): R_{f} 0.29 (50% EtOAc/Hexanes); [α]_{D}²⁰ 1.3 (c 1.0, CHCl₃); ¹H NMR (400 MHz, CDCl₃) δ 1.75-1.95 (m, 1H, 4-HH), 2.51 (m, 1H, 4-HH), 3.25 (dd, 2H, J 15.6, 11.2, 5-H₂), 3.45 (m, 2H, 3′-H₂), 3.78 (s, 3H, OMe), 4.05-4.21 (m, 2H, Fmoc-H and 3-H), 4.35 (d, 2H, J 6.4, Fmoc-H₂), 5.20 (dd, 1H, J 11.2, 4.2, 2′H), 5.40 (d, 1H, J 4.0, 3-NH), 7.00 (br s, 1H, 2-NH), 7.11-7.42 (m, 12H, ar-H) and 8.12 (br s, 1H, NH-Trp); ¹³C NMR (100 MHz, CDCl₃) δ 25.2 (CH₂), 27.5 (CH₂), 41.4 (CH₂), 47.3 (CH₃), 52.8 (CH), 54.6 (CH), 67.3 (CH₂), 110.6 (ar-C), 111.5 (ar-CH), 116.8 (CH), 118.4 (ar-CH), 119.9 (ar-CH), 120.1 (ar-CH), 122.0 (ar-CH), 122.6 (ar-CH), 125.3 (ar-CH), 127.3 (ar-CH), 127.9 (ar-CH), 136.3 (ar-C), 141.5 (ar-C), 144.0 (ar-C), 144.1 (ar-C) and 171.1 (C═O); υₘₐₓ/cm⁻¹ (NaCl): 3335 (NH), 2952 (CH), 1692 (CO), 1524, 1450, 1248, 1053 and 741; HRMS Calcd. for C₃₁H₃₀O₅N₃ [M+H]⁺ 524.2180. found 524.2191.

### Example 19

### (S)-Methyl-2-{(S)-3-[N-(9-fluorenylmethoxycarbonyl)amino]-2-oxopyrrolidin-1-yl}-3-(1H-indol-3-yl)propanoate (13)

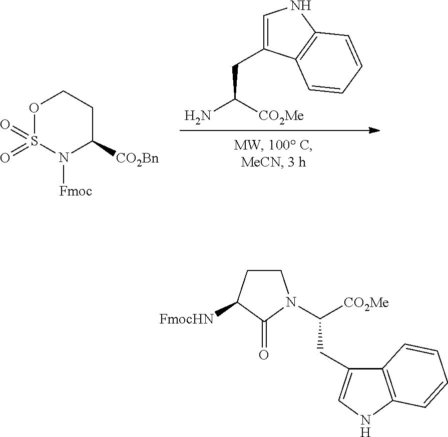

Tryptophan methyl ester hydrochloride (133 mg, 0.61 mmol) was converted to its free amino ester as described above, dissolved in acetonitrile (2 mL) and the solution was added to (4S)-benzyl-2,2-dioxo-3-(9-fluorenylmethoxycarbonyl)-1,2,3-oxathiazinane-4-carboxylate [(4S)-8, 100 mg, 0.2 mmol) in a 2 mL microwave vessel. The reaction mixture was heated to 100° C. under microwave irradiation for 3 h and treated with 1M mono-potassium phosphate (4 mL). The two layers were separated and the aqueous layer was extracted with EtOAc (4×10 mL). The combined organic layers were dried (MgSO₄) and concentrated to a solid, that was purified by flash column chromatography (30% EtOAc/hexanes) to give (S)-methyl 2-{(S)-3-[N-(9-fluorenylmethoxycarbonyl)amino]-2-oxopyrrolidin-1-yl}-3-(1H-indol-3-yl)propanoate (13, 56 mg, 54%) as a white foam.

### Example 20

### (S)-Methyl-2-{(S)-4-[N-(9-fluorenylmethoxycarbonyl)amino]-2-oxopyrrolidin-1-yl}-3-(1H-indol-3-yl)propanoate (18)

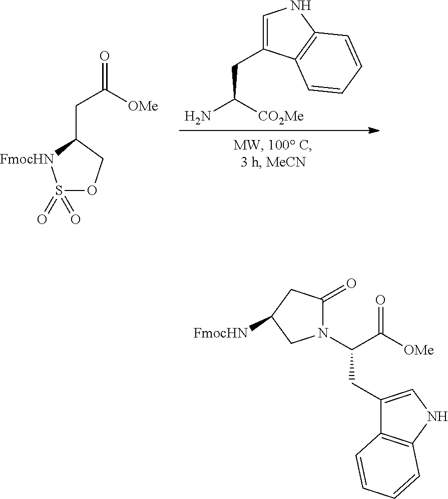

Tryptophan methyl ester hydrochloride (157 mg, 0.72 mmol) was converted to its free amino ester as described above, dissolved in acetonitrile (2 mL) and the solution was added to (4S)-methyl-2-[3-(9-fluorenylmethoxycarbonyl)-2,2-dioxo-1,2,3-oxathiazolidin-4-yl]acetate (17, 100 mg, 0.24 mmol) in a 2 mL microwave vessel. The reaction mixture was heated to 100° C. under microwave irradiation for 3 h and treated with 1M mono-potassium phosphate (4 mL). The layers were separated. The aqueous layer was extracted with EtOAc (4×10 mL). The combined organic layers were dried (MgSO₄) and concentrated using a rotary evaporator to give a yellow oil, that was purified by flash column chromatography (60% EtOAc/hexanes) to yield (S)-methyl 2-{(S)-4-[N-(9-fluorenylmethoxycarbonyl)amino]-2-oxopyrrolidin-1-yl}-3-(1H-indol-3-yl)propanoate (18, 98 mg, 78%) as a white foam: R_{f} 0.26 (80% EtOAc/Hexanes); [α]²³_{D} 20.5 (c=1.95 in CHCl₃); ¹H NMR (300 MHz, acetone-d₆) δ 2.28 (dd, 1H, CHH—C═O lactam, J=5.6, 16.7), 2.62 (dd, 1H, CHH—C═O lactam, J=8.1, 16.7), 3.22 (dd, 1H, CHH Trp, J=10.1, 15.0), 3.37 (m, 1H, CHH—N lactam), 3.42 (dd, 1H, CHH Trp, J=5.4, 15.0), 3.69 (s, 3H, CH₃), 3.74 (dd, 1H, CHH—N lactam, J=9.2, 16.8), 4.19 (t, 1H, Fmoc-CH, J=6.8), 4.28 (m, 1H, CH—N), 4.33 (d, 2H, Fmoc-CH₂, J=6.8), 5.13 (dd, 1H, ⊕-H Trp, J=5.7, 9.6), 6.68 (d, 1H, NH, J=6.1), 7.02 (td, 1H, H₅ indole, J=1.2, 6.9), 7.11 (td, 1H, H₆ indole, J=1.2, 6.9), 7.18 (s, 1H, H₂ indole), 7.30 (t, 2H, 2×ar-H Fmoc, J=7.1), 7.40 (t, 2H, 2×ar-H Fmoc, J=7.1), 7.37 (d, 1H, H₇ indole, J=7.8), 7.61 (d, 1H, H₄ indole, J=7.5), 7.64 (d, 2H, 2×ar-H Fmoc, J=7.4), 7.84 (d, 2H, 2×ar-H Fmoc, J=7.5), 10.01 (s, 1H, NH indole); ¹³C NMR (75 MHz, acetone-d₆) δ 25.6 (CH₂), 37.7 (CH₂), 45.9 (CH), 48.0 (CH), 51.1 (CH₂), 52.4 (CH₃), 54.7 (CH), 66.8 (CH₂), 109.9 (ar-CH), 111.5 (ar-CH), 118.3 (ar-CH), 119.7 (ar-CH), 120.1 (ar-CH), 122.2 (ar-CH), 122.4 (ar-CH), 124.9 (ar-CH), 126.7 (ar-CH), 127.3 (ar-C), 128.0 (ar-C), 136.0 (ar-C), 141.4 (ar-C), 143.8 (ar-C), 155.6 (C═O), 171.1 (C═O), 173.2 (C═O); HRMS calcd. for C₃₁H₃₀N₃O₅ [M+H]⁺ : 524.2180. found: 524.2180; υₘₐₓ/cm⁻¹ (NaCl): 3432 (N—H), 3019 (C—H arom.), 1738, 1714, 1693 (C═O), 1514, 1479, 1450 (C═C arom.), 1435 (C—H alkyl), 1213 (C—O ester), and 753.

### Solid-Phase Peptide Synthesis General Experimental Procedures

### Example 21

### Rink Resin Swelling and Deprotection

A 12 mL plastic filtration tube with a polyethylene filter was charged with Rink resin (300 mg, 0.09 mmol, 0.3 mmol/g) or Rink MBHA resin (300 mg, 0.21 mmol, 0.7 mmol/g) and DMF (7 mL). The tube was sealed and shaken for 0.5 h. The resin was then filtered and taken up in freshly prepared 20% piperidine in DMF solution (7 mL), shaken for 30 min, filtered, retreated with 20% piperidine/DMF solution (7 mL) and shaken for 30 min. The resin was washed by successive agitations for 1 min and filtered from DMF (3×7 mL), MeOH (3×7 mL) and DCM (3×7 mL). A positive Kaiser colour test indicated qualitatively the presence of free amine.^{lix}

### Example 22

### Amino Acid Couplings

The resin was first swollen in DMF (7 mL) for 15 min. Meanwhile, a solution of N-(Fmoc)amino acid (Fmoc-Xaa-OH, 3 equiv.), HBTU (3 equiv.) and DIEA (6 equiv.) in DMF (7 mL) was prepared in a small sample vial, stirred for 10 min and then added to the resin. The reaction mixture was shaken for 1 h with Fmoc-Lys(Boc)-OH and Fmoc-Ala-OH), 3 h with Fmoc-Phe-OH, Fmoc-Trp(Boc)-OH, Fmoc-His(Trt)-OH and Fmoc-D-Val-OH), and 4 h with Fmoc-D-Leu-OH, Fmoc-D-Glu(^{t}Bu)-OH, Fmoc-D-Thr(^{t}Bu)-OH, Fmoc-D-Tyr(^{t}Bu)-OH and Fmoc-D-Arg(Pbf)-OH, at room temperature. The resin was filtered and respectively washed by shaking for 1 min with DMF (3×7 mL), MeOH (3×7 mL) and DCM (3±7 mL). A negative Kaiser test response indicated completion of the reaction. The resin was then dried in vacuo.^{lix}

### Example 23

### Silylation and Alkylation

After swelling the resin, the Fmoc protecting group was removed as described above. The resin was then dried in vacuo for at least 3 h. The anhydrous resin, in a 12 mL plastic filtration tube with polyethylene frit, was then flushed with argon, swollen in THF (7 mL), treated with BSA (5 equiv.), shaken for 16 h, filtered under argon and treated with a solution of sulfamidate (5 equiv.) in THF (7 mL). After shaking for 24 h, the resin was filtered and washed under argon with THF (3×7 mL), MeOH (3×7 mL) and DCM (3×7 mL) and dried in vacuo.

### Example 24

### Microwave Assisted Annulation

A 2 mL glass microwave vial was charged with resin and either DMF (2 mL) (conditions A) or a freshly prepared 1% acetic acid/DMSO solution (2 mL) (conditions B). The vial was sealed, heated in the microwave at 100° C. (pressure 1 bar) for 75 min-10 h (as specified) and then cooled using a jet of air. The resin was then washed from the microwave vessel into a 12 mL plastic filtration tube with polyethylene frit and washed by shaking for 1 min with DMF (3×7 mL), MeOH (3×7 mL) and DCM (3×7 mL) and then dried in vacuo.

### Example 25

### Resin Capping

The resin was swollen in a solution of di-tert-butyl dicarbonate (5 equiv.) in DMF (7 mL), treated with DIEA (10 equiv.), shaken for 1 h, filtered and washed by shaking for 1 min with DMF (3×7 mL), MeOH (3×7 mL) and DCM (3×7 mL) and then dried in vacuo.

### Example 26

### Peptide Cleavage

The resin was first swollen in DCM in a plastic filtration tube with polyethylene frit, as described for Fmoc removal above, treated with a freshly prepared 20% piperidine/DMF solution (7 mL), shaken for 15 min, filtered, treated with a second portion of 20% piperidine/DMF solution (7 mL) and shaken for 15 min. The resin was then filtered and washed by shaking for 1 min with DMF (3×7 mL), MeOH (3×7 mL) and DCM (3×7 mL). A positive Kaiser colour test indicated qualitatively the presence of free amine.^{lix} The peptide was then cleaved from the resin by shaking in TFA/H₂O/TES (7 mL, 95/2.5/2.5, v/v/v) for 2 h. The resin was filtered, washed with TFA (7 mL) and the combined filtrate and washings were concentrated in vacuo. The resulting residue was dissolved in a minimum volume of TFA (˜1 mL), transferred to a centrifuge tube and precipitated by the addition of ice-cold diethyl ether (40 mL). The peptide was then centrifuged and the diethyl ether was carefully decanted from the tube. The treatment of the precipitated peptide with cold diethyl ether wash was repeated twice. The resulting white solid was dissolved in water (10 mL) and freeze-dried to give a white foam that was purified by preparatory RP-HPLC, using the specified conditions.

### Example 27

### Tripeptides for Optical Purity Study

### (a) Ala-(S)-Agl-Ala-NH₂ (34)

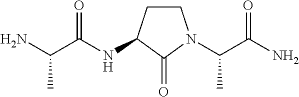

Ala-(S)-Agl-Ala-NH₂ 34 was prepared as described above to give the desired lactam-bridged tripeptide TFA salt (28 mg, 59% crude as assessed by RP-HPLC [Gemini™ column from Phenomenex (150 mm×4.6 mm, 5 μm, C₁₈) with a flow rate of 0.5 mL/min using a linear gradient 20-80% MeCN (0.1% FA) in water (0.1% FA) over 30 min, t_{R} 14.92). Purification was then carried out by preparatory RP-HPLC (5-90% MeCN (0.1% TFA), 10 min gradient) to give the desired TFA salt product 34 (12 mg, 16%) as a brown foam. [∝_{D}²⁰ −20.6 (c 1.0, H₂O); ¹H NMR (400 MHz, D₂O) δ 1.43 (d, 3H, J 7.4, ∝₃-CH₃), 1.55 (d, 3H, J 7.1, ∝₁-CH₃), 2.02-2.12 (m, 1H, (β₂—CHH), 2.49-2.57 (m, 1H, β₂—CHH), 3.51-3.56 (m, 2H, ∝₂-CH₂), 4.10 (q, 1H, J 7.1, ∝₃-CH), 4.56 (t, 1H, J 9.6, ∝₂-CH), 4.63 (q, 1H, J 7.4, ∝₁-CH); ¹³C NMR (100 MHz, D₂O) δ 13.5 (CH₃), 16.3 (CH₃), 24.5 (CH₂), 41.6 (CH₂), 48.9 (CH), 51.3 (CH), 51.7 (CH), 170.7 (C═O), 174.0 (C═O), 175.6 (C═O); υₘₐₓ/cm⁻¹ (KBr): 3421 (NH), 2919 (CH), 2360, 1672 (CO), 1439, 1204, 1134, 933, 838, 801 and 722; HRMS Calcd. for C₁₀H₁₉O₃N₄ [M+H]⁺ 243.1452. found 243.1456.

### (b) Ala-(R)-Agl-Ala-NH₂ (35)

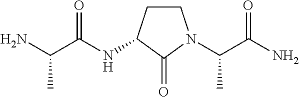

Ala-(R)-Agl-Ala-NH₂ 35 was prepared as described above to give the desired lactam-bridged tripeptide TFA salt (36 mg, 90% crude purity crude as assessed by RP-HPLC [Gemini™ column from Phenomenex (150 mm×4.6 mm, 5 μm, C₁₈) with a flow rate of 0.5 mL/min using a linear gradient 20-80% MeCN (0.1% FA) in water (0.1% FA) over 30 min, t_{R} 14.95). Purification was then carried out by preparatory RP-HPLC (5-90% MeCN (0.1% TFA), 10 min gradient) to give the desired formic acid salt 35 (11 mg, 15%) as a brown foam. [α]_{D}²⁰ +3.4 (c 1.0, H₂O); ¹H NMR (400 MHz, D₂O) δ 1.45 (d, 3H, J 7.3, ∝₃-CH₃), 1.53 (d, 3H, J 7.1, ∝₁-CH₃), 1.98-2.09 (m, 1H, β₂-CHH), 2.49-2.56 (m, 1H, β₂-CHH), 3.47 (dt, 1H, J 9.6, 7.5, ∝₂-CHH), 3.61 (td, 1H, J 8.2, 1.5, ∝₂-CHH), 4.09 (q, 1H, J 7.1, ∝₃-CH), 4.61 (t, 1H, J 6.1, ∝₂-CH), 4.63 (q, 1H, J 7.1, ∝₁-CH); ¹³C NMR (100 MHz, D₂O) δ 13.5 (CH₃), 16.3 (CH₃), 24.9 (CH₂), 41.5 (CH₂), 48.9 (CH), 51.4 (CH), 51.8 (CH), 170.7 (C═O), 173.9 (C═O), 175.3 (C═O); υₘₐₓ/cm⁻¹ (KBr): 3427 (NH), 2996 (CH), 2406, 1674 (CO), 1440, 12041135, 936, 838 and 801; HRMS Calcd. for C₁₀H₁₉O₃N₄ [M+H]⁺ 243.1452. found 243.1463.

### Example 28

### GHRP-6 (S)-Agl Analogs

### (a) (S)-Agl-D-Trp-Ala-Trp-D-Phe-Lys-NH₂ (44)

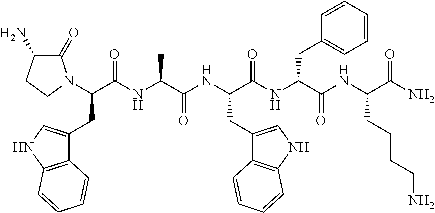

(S)-Agl-D-Trp-Ala-Trp-D-Phe-Lys-NH₂ 44 was prepared as described above, using microwave assisted annulation conditions A over 90 min, to give the desired lactam peptide TFA salt 44 (101 mg, 30% crude purity by analytical RP-HPLC (UV 214), 10-30% MeCN in H₂O, 0.1% FA, 15 min gradient). Purification was then carried out by preparatory RP-HPLC (10-30% MeCN in H₂O, 0.1% FA, 30 min gradient) to give the desired formic acid salt product 44 (6 mg, 8%) as a white foam. The purified product was analyzed by analytical RP-HPLC (UV 214) using both MeCN t_{R} 11.19 (0-60% MeCN in H₂O, 0.1% FA, 30 min gradient) and MeOH t_{R} 14.50 (0-60% MeOH in H₂O, 0.1% FA, 30 min gradient) and revealed >99% purity. HRMS Calcd. for C₄₄H₅₅O₆N₁₀ [M+H]⁺ 819.43006. found 819.42910.

### (b) His-(S)-Agl-Ala-Trp-D-Phe-Lys-NH₂ (45)

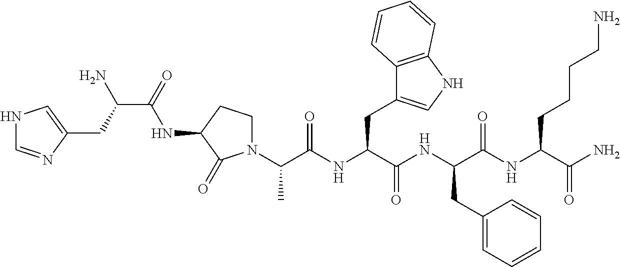

His-(S)-Agl-Ala-Trp-D-Phe-Lys-NH₂ 45 was prepared as described above, using microwave assisted annulation conditions A over 3 h, to give the desired lactam-bridged peptide TFA salt (66 mg, 36% crude purity by analytical RP-HPLC (UV 214), 0-40% MeCN in H₂O, 0.1% FA, 15 min gradient). Purification by preparatory RP-HPLC (0-40% MeCN in H₂O, 0.1% FA, 30 min gradient) then gave formic acid salt 45 (16 mg, 22%) as a white foam. The purified product was analyzed by analytical RP-HPLC (UV 214) using both MeCN t_{R} 4.50 (10-40% MeCN in H₂O, 0.1% FA, 15 min gradient) and MeOH t_{R} 7.83 (10-40% MeOH in H₂O, 0.1% FA, 15 min gradient) and revealed >99% purity. HRMS Calcd. for C₃₉H₅₃O₆N₁₁ [M+2H]²⁺ 385.7085. found 385.7094.

### (c) His-D-Trp-(S)-Agl-Trp-D-Phe-Lys-NH₂ (46)

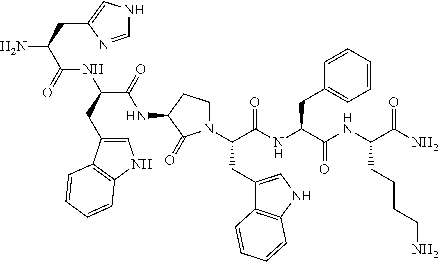

His-D-Trp-(S)-Agl-Trp-D-Phe-Lys-NH₂ 46 was prepared as described above, using microwave assisted annulation conditions A over 75 min, to give the desired lactam-bridged peptide TFA salt 46 (83 mg, 22% crude purity as analyzed by analytical RP-HPLC, O-20 MeCN, 8 min gradient). Purification by preparatory RP-HPLC (10-40% MeCN in H₂O, 0.1% FA, 20 min gradient) then gave the formic acid salt 46 (9.5 mg, 11%) as a white foam. The purified product was analyzed by analytical RP-HPLC (UV 214) using both MeCN t_{R} 8.24 (10-40% MeCN in H₂O, 0.1% FA, 15 min gradient) and MeOH t_{R} 8.28 (10-40% MeOH in H₂O, 0.1% FA, 15 min gradient) and revealed >99% purity. HRMS Calcd. for C₄₇H₅₈O₆N₁₂ [M+2H]²⁺ 443.2304. found 443.2296.

### (d) His-D-Trp-Ala-(S)-Agl-D-Phe-Lys-NH₂ (47)

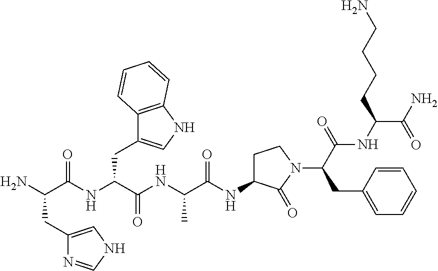

His-D-Trp-Ala-(S)-Agl-D-Phe-Lys-NH₂ 47 was prepared as described above, using microwave assisted annulation conditions A over 75 min, to give the desired lactam-bridged peptide TFA salt (59 mg, 66% crude purity as judged by analytical RP-HPLC, O-40% MeCN in H₂O, 0.1% FA, 15 min gradient). Purification was then carried out by preparatory RP-HPLC (0-40% MeCN in H₂O, 0.1% FA, 30 min gradient) to give the desired formic acid salt (9 mg, 11%) as a white foam. The purified product was analyzed by analytical RP-HPLC (UV 214) using both MeCN t_{R} 14.37 (5-40% MeCN in H₂O, 0.1% FA, 15 min gradient) and MeOH t_{R} 11.15 (5-40% MeOH in H₂O, 0.1% FA, 15 min gradient) and revealed >99% purity. HRMS Calcd. for C₃₉H₅₂O₆N₁₁ [M+H]⁺ 770.4097. found 770.4090.

### (e) His-D-Trp-Ala-Trp-(S)-Agl-Lys-NH₂ (48)

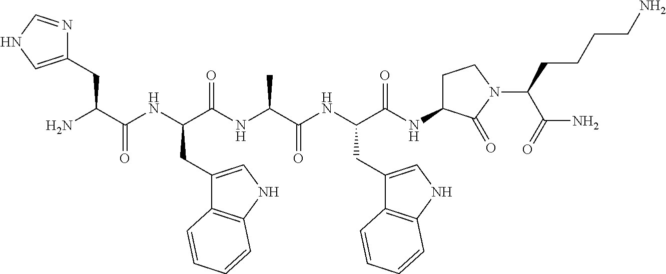

His-D-Trp-Ala-Trp-(S)-Agl-Lys-NH₂ 48 was prepared as described above, using microwave assisted annulation conditions A over 75 min, to give the desired lactam-bridged peptide TFA salt (64 mg) of 44% crude purity as analyzed by analytical RP-HPLC (0-20 MeCN, 4 min gradient). Purification was then carried out by preparatory RP-HPLC (0-20 MeCN, 30 min gradient) to give the desired formic acid salt (10 mg, 13%), as a white foam. The purified product was analyzed by analytical RP-HPLC (UV 214) using both MeCN t_{R} 8.92 (0-60% MeCN in H₂O, 0.1% FA, 15 min gradient) and MeOH t_{R} 10.41 (0-60% MeOH in H₂O, 0.1% FA, 15 min gradient) and revealed to be of >99% purity. HRMS Calcd. for C₄₁H₅₄O₆N₁₂ [M+2H]²⁺ 405.2139. found 405.2149.

### Example 29

### GHRP-6 L-Bgl Analogs

### (a) (S)-Bgl-D-Trp-Ala-Trp-D-Phe-Lys-NH₂ (49)

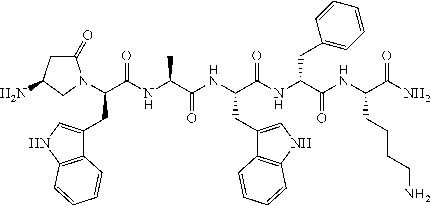

(S)-Bgl-D-Trp-Ala-Trp-D-Phe-Lys-NH₂ 49 was prepared was prepared as described above, using microwave assisted annulation conditions A over 180 min to give the desired lactam peptide TFA salt 49 (68 mg, 39% crude purity by analytical RP-HPLC (UV 214), 0-40% MeCN in H₂O, 0.1% FA, 15 min gradient). Purification was then carried out by preparatory RP-HPLC (10-30% MeCN in H₂O, 0.1% FA, 30 min gradient) to give the desired formic acid salt 49 (14 mg, 22%) as a white foam. The purified product was analyzed by analytical RP-HPLC (UV 214) using both MeCN t_{R} 15.47 (0-60% MeCN in H₂O, 0.1% FA, 30 min gradient) and MeOH t_{R} 21.14 (0-60% MeOH in H₂O, 0.1% FA, 30 min gradient) and revealed 92% purity. HRMS Calcd. for C₄₄H₅₅O₆N₁₀ [M+H]⁺ 819.4301. found 819.4298.

### (b) His-(S)-Bgl-Ala-Trp-D-Phe-Lys-NH₂ (50)

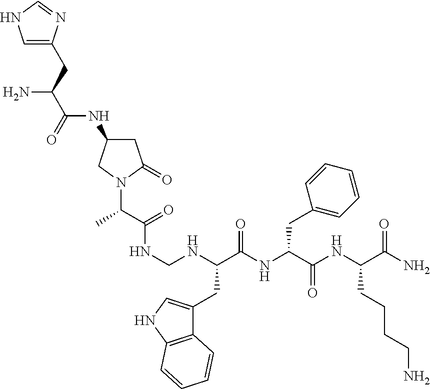

His-(S)-Bgl-Ala-Trp-D-Phe-Lys-NH₂ 50 was prepared was prepared as described above, using microwave assisted annulation conditions A over 180 min to give the desired lactam peptide TFA salt 50 (71 mg, 26% crude purity by analytical RP-HPLC (UV 214), 0-40% MeCN in H₂O, 0.1% FA, 15 min gradient). Purification was then carried out by preparatory RP-HPLC (10-40% MeCN in H₂O, 0.1% FA, 30 min gradient) to give the desired formic acid salt product 50 (8 mg, 12%) as a white foam. The purified product was analyzed by analytical RP-HPLC (UV 214) using both MeCN t_{R} 10.45 (0-60% MeCN in H₂O, 0.1% FA, 30 min gradient) and MeOH t_{R} 12.57 (0-60% MeOH in H₂O, 0.1% FA, 30 min gradient) and revealed 91% purity. HRMS Calcd. for C₃₉H₅₂O₆N₁₁ [M+H]⁺ 770.4097. found 770.4097.

### (c) His-D-Trp-(S)-Bgl-Trp-D-Phe-Lys-NH₂ (51)

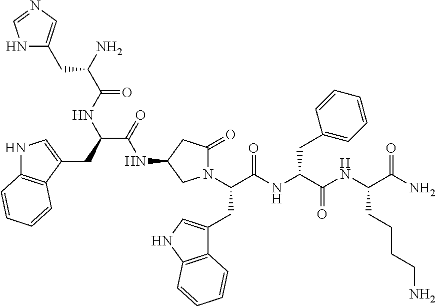

His-D-Trp-(S)-Bgl-Trp-D-Phe-Lys-NH₂ 51 was prepared as described above, using microwave assisted annulation conditions A over 180 min to give the desired lactam peptide TFA salt 51 (48 mg, 46% crude purity by analytical RP-HPLC (UV 214), 0-40% MeCN in H₂O, 0.1% FA, 15 min gradient). Purification was then carried out by preparatory RP-HPLC (10-40% MeCN in H₂O, 0.1% FA, 30 min gradient) to give the desired formic acid salt 51 (19 mg, 25%) as a white foam. The purified product was analyzed by analytical RP-HPLC (UV 214) using both MeCN t_{R} 12.75 (10-40% MeCN in H₂O, 0.1% FA, 20 min gradient) and MeOH t_{R} 18.40 (0-60% MeOH in H₂O, 0.1% FA, 30 min gradient) and revealed >99% purity. HRMS Calcd. for C₄₇H₅₇O₆N₁₂ [M+H]⁺ 885.4519. found 885.4511.

### (d) His-D-Trp-Ala-(S)-Bgl-D-Phe-Lys-NH₂ (52)

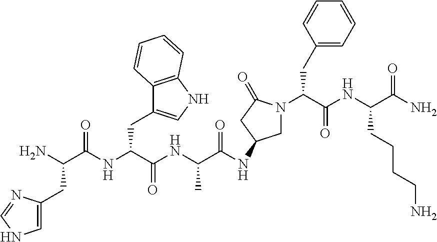

His-D-Trp-Ala-(S)-Bgl-D-Phe-Lys-NH₂ 52 was prepared was prepared as described above, using microwave assisted annulation conditions A over 180 min to give the desired lactam peptide TFA salt 52 (26 mg, 52% crude purity by analytical RP-HPLC (UV 214), 0-40% MeCN in H₂O, 0.1% FA, 15 min gradient). Purification was then carried out by preparatory RP-HPLC (10-35% MeCN in H₂O, 0.1% FA, 30 min gradient) to give the desired formic acid salt product 52 (4 mg, 6%) as a white foam. The purified product was analyzed by analytical RP-HPLC (UV 214) using both MeCN t_{R} 10.61 (0-60% MeCN in H₂O, 0.1% FA, 30 min gradient) and MeOH t_{R} 13.39 (0-60% MeOH in H₂O, 0.1% FA, 30 min gradient) and revealed >99% purity. HRMS Calcd. for C₃₉H₅₂O₆N₁₁ [M+H]⁺ 770.4097. found 770.4089.

### (e) His-D-Trp-Ala-Trp-(S)-Bgl-Lys-NH₂ (53)

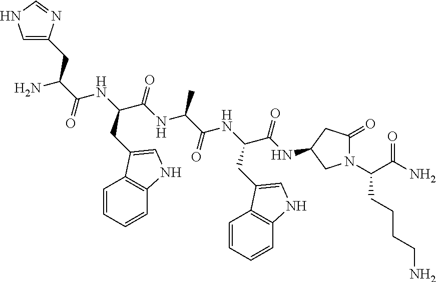

His-D-Trp-Ala-Trp-(S)-Bgl-Lys-NH₂ 53 was prepared was prepared as described above, using microwave assisted annulation conditions A over 180 min, to give the desired lactam peptide TFA salt 53 (49 mg, 92% crude purity by analytical RP-HPLC (UV 214), 0-40% MeCN in H₂O, 0.1% FA, 15 min gradient). Purification was then carried out by preparatory RP-HPLC (0-40% MeCN in H₂O, 0.1% FA, 25 min gradient) to give the desired formic acid salt 53 (3 mg, 5%) as a white foam. The purified product was analyzed by analytical RP-HPLC (UV 214) using both MeCN t_{R} 4.59 (0-60% MeCN in H₂O, 0.1% FA, 30 min gradient) and MeOH t_{R} 10.23 (0-60% MeOH in H₂O, 0.1% FA, 30 min gradient) and revealed >99% purity. HRMS Calcd. for C₄₁H₅₃O₆N₁₂ [M+H]⁺ 809.4199. found 831.4019.

### Example 30

### 101.10 D-Agl Analogs

### (a) (R)-Agl-D-Tyr-D-Thr-D-Val-D-Glu-D-Leu-D-Ala-NH₂ (54)

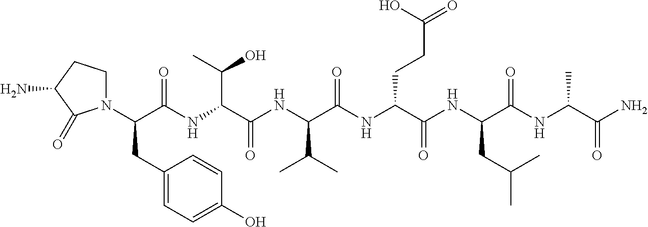

(R)-Agl-D-Tyr-D-Thr-D-Val-D-Glu-D-Leu-D-Ala-NH₂ 54 was prepared as described above, using microwave assisted annulation conditions B over 8 h, to give the desired lactam peptide TFA salt 59 (57 mg, 55% crude purity as analyzed by analytical RP-HPLC (UV 214), 2-40% MeCN in H₂O, 0.1% FA, 15 min gradient). Purification was then carried out by preparatory RP-HPLC (2-20% MeCN in H₂O, 0.1% FA, 20 min gradient) to give the desired formic acid salt 54 (16 mg, 20%) as a white foam. The purified product was analyzed by analytical RP-HPLC (UV 214) using both MeCN t_{R} 10.98 (0-60% MeCN in H₂O, 0.1% FA, 30 min gradient) and MeOH t_{R} 15.30 (0-60% MeOH in H₂O, 0.1% FA, 30 min gradient) and revealed >99% purity. HRMS Calcd. for C₃₆H₅₇O₁₁N₈ [M+H]⁺ 777.4141. found 777.4138.

### (b) D-Arg-(R)-Agl-D-Thr-D-Val-D-Glu-D-Leu-D-Ala-NH₂ (55)

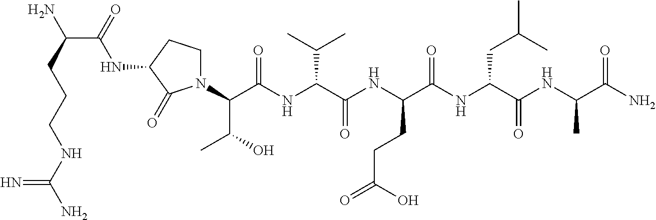

D-Arg-(R)-Agl-D-Thr-D-Val-D-Glu-D-Leu-D-Ala-NH₂ 55 was prepared as described above, using microwave assisted annulation conditions B over 10 h, to give the desired lactam peptide TFA salt 55 (99 mg, 41% crude purity as analyzed by analytical RP-HPLC (UV 214), 0-20% MeCN in H₂O, 0.1% FA, 15 min gradient). Purification was then carried out by preparatory RP-HPLC (0-20% MeCN in H₂O, 0.1% FA, 30 min gradient) to give the desired formic acid salt 55 (22 mg, 15%) as a white foam. The purified product was analyzed by analytical RP-HPLC (UV 214) using both MeCN t_{R} 8.26 (0-60% MeCN in H₂O, 0.1% FA, 30 min gradient) and MeOH t_{R} 9.36 (0-60% MeOH in H₂O, 0.1% FA, 30 min gradient) and revealed >99% purity. HRMS Calcd. for C₃₃H₆₀O₁₀N₁₁ [M+H]⁺ 770.4519. found 770.4514.

### (c) D-Arg-D-Tyr-(R)-Agl-D-Val-D-Glu-D-Leu-D-Ala-NH₂ (56)

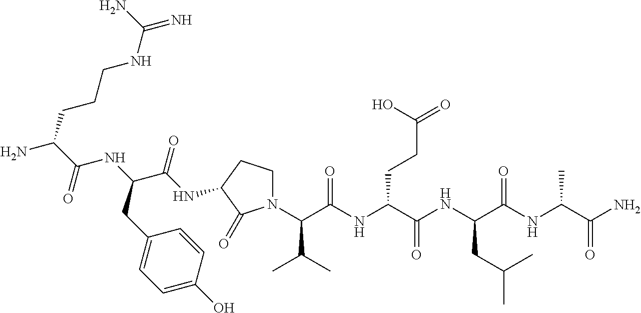

D-Arg-D-Tyr-(R)-Agl-D-Val-D-Glu-D-Leu-D-Ala-NH₂ 56 was prepared as described above, using microwave assisted annulation conditions B over 6 h, to give the desired lactam peptide TFA salt 56 (89 mg, 58% crude purity as analyzed by analytical RP-HPLC (UV 214), 0-40% MeCN in H₂O, 0.1% FA, 20 min gradient). Purification was then carried out by preparatory RP-HPLC (0-40% MeCN in H₂O, 0.1% FA, 30 min gradient) to give the desired formic acid salt 56 (13 mg, 7%) as a white foam. The purified product was analyzed by analytical RP-HPLC (UV 214) using both MeCN t_{R} 9.84 (0-60% MeCN in H₂O, 0.1% FA, 30 min gradient) and MeOH t_{R} 12.65 (0-60% MeOH in H₂O, 0.1% FA, 30 min gradient) and revealed >99% purity. HRMS Calcd. for C₃₈H₆₂O₁₀N₁₁ [M+H]⁺ 832.4676. found 832.4676.

### (d) D-Arg-D-Tyr-D-Thr-(R)-Agl-D-Glu-D-Leu-D-Ala-NH₂ (57)

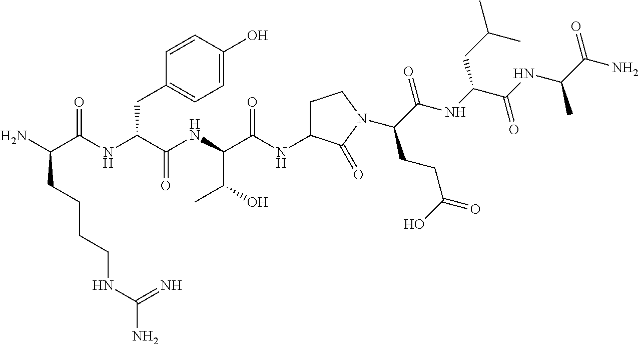

D-Arg-D-Tyr-D-Thr-(R)-Agl-D-Glu-D-Leu-D-Ala-NH₂ 57 was prepared as described above, using microwave assisted annulation conditions B over 4 h, to give the desired lactam peptide TFA salt 57 (79 mg, 50% crude purity as analyzed by analytical RP-HPLC (UV 214), 2-40% MeCN in H₂O, 0.1% FA, 15 min gradient). Purification was then carried out by preparatory RP-HPLC (2-20% MeCN in H₂O, 0.1% FA, 20 min gradient) to give the desired formic acid salt 57 (21 mg, 11%) as a white foam. The purified product was analyzed by analytical RP-HPLC (UV 214) using both MeCN t_{R} 9.19 (0-60% MeCN in H₂O, 0.1% FA, 30 min gradient) and MeOH t_{R} 11.15 (0-60% MeOH in H₂O, 0.1% FA, 30 min gradient) and revealed >99% purity. HRMS Calcd. for C₃₇H₆₀O₁₁N₁₁ [M+H]⁺ 833.4468. found 833.4459.

### (e) D-Arg-D-Tyr-D-Thr-D-Val-(R)-Agl-D-Leu-D-Ala-NH₂ (58)

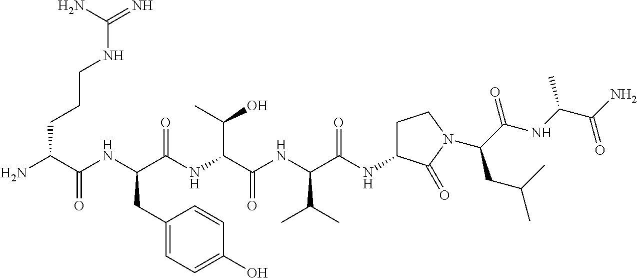

D-Arg-D-Tyr-D-Thr-D-Val-(R)-Agl-D-Leu-D-Ala-NH₂ 58 was prepared as described above, using microwave assisted annulation conditions B over 4 h, to give the desired lactam peptide TFA salt 58 (157 mg, 36% crude purity as analyzed by analytical RP-HPLC (UV 214), 0-40% MeCN in H₂O, 0.1% FA, 30 min gradient). Purification was then carried out by preparatory RP-HPLC (0-40% MeCN in H₂O, 0.1% FA, 30 min gradient) to give the desired formic acid salt 58 (37 mg, 21%) as a white foam. The purified product was analyzed by analytical RP-HPLC (UV 214) using both MeCN t_{R} 9.93 (0-60% MeCN in H₂O, 0.1% FA, 30 min gradient) and MeOH t_{R} 12.57 (0-60% MeOH in H₂O, 0.1% FA, 30 min gradient) and revealed >99% purity. HRMS Calcd. for C₃₇H₆₂O₉N₁₁ [M+H]⁺ 803.4727. found 803.4725.

### (f) D-Arg-D-Tyr-D-Thr-D-Val-D-Glu-(R)-Agl-D-Ala-NH₂ (59)

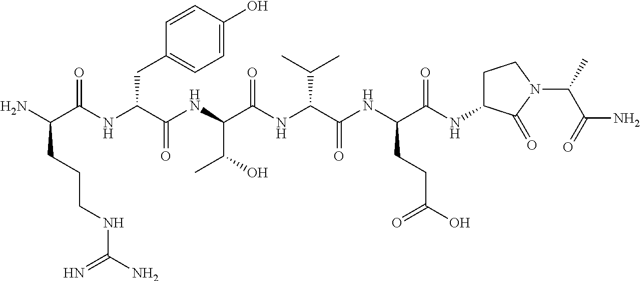

D-Arg-D-Tyr-D-Thr-D-Val-D-Glu-(R)-Agl-D-Ala-NH₂ 59 was prepared as described above, using microwave assisted annulation conditions B over 3 h, to give the desired lactam peptide TFA salt 59 (70 mg, 81% crude purity as analyzed by analytical RP-HPLC (UV 214), 0-40% MeCN in H₂O, 0.1% FA, 4 min gradient). Purification was then carried out by preparatory RP-HPLC (0-40% MeCN in H₂O, 0.1% FA, 20 min gradient) to give the desired formic acid salt 59 (2.6 mg, 7%) as a white foam. The purified product was analyzed by analytical RP-HPLC (UV 214) using both MeCN t_{R} 8.52 (0-60% MeCN in H₂O, 0.1% FA, 30 min gradient) and MeOH t_{R} 9.91 (0-60% MeOH in H₂O, 0.1% FA, 30 min gradient) and revealed >99% purity. HRMS Calcd. for C₃₆H₅₈O₁₁N₁₁ [M+H]⁺ 820.4312. found 820.4307.

Discussion for Examples 1-30

With five- and six-member cyclic sulfamidates 17 and 8 in hand, and their effectiveness in solution-phase chemistry demonstrated, the solid-phase synthesis of Agl and Bgl peptides was pursued on Rink amide resin. N-Alkylation was initially tested on Lys(omega-Boc) bound to Rink resin 19 using sulfamidate 8 (200 mol %) in THF for 12 h at room temperature; however, low amounts of the desired product were observed by LC-MS due to bisalkylation of the amine. Several strategies to overcome the issue of bisalkylation were examined without success, including varying reaction temperature, sulfamidate stoichiometry and rate of addition, as well as adding acid to protonate the secondary amine.

Attention turned towards o-nitrobenzenesulfonamide as a temporary protecting group. Resin-bound Lys(omega-Boc) 19 was reacted with o-nitrobenzenesulfonyl chloride (o-NBS-Cl) and 2,4,6-collidine in DCM to give o-nitrobenzenesulfonamide 20 (Scheme 3). Attempts to N-alkylate 20 using sulfamidate 8 (500 mol %) in THF for 24 h gave incomplete reactions. On the other hand, Mitsunobu reaction of nitrophenylsulfonamide 20 with Fmoc-Hse-OBn 10b using triphenyl phosphine and diisopropyl azodicarbonate (DIAD) in THF proceeded quantitatively in 24 h as determined by LC-MS analysis. Removal of the o-nitrobenzenesulfonyl group from N-alkyl sulfonamide 21 using different thiols and conditions failed, however, likely due to the use of the Rink amide linker, which was previously suggested to prevent o-NBS removal during the synthesis of diketopiperazines.^{lxiii}

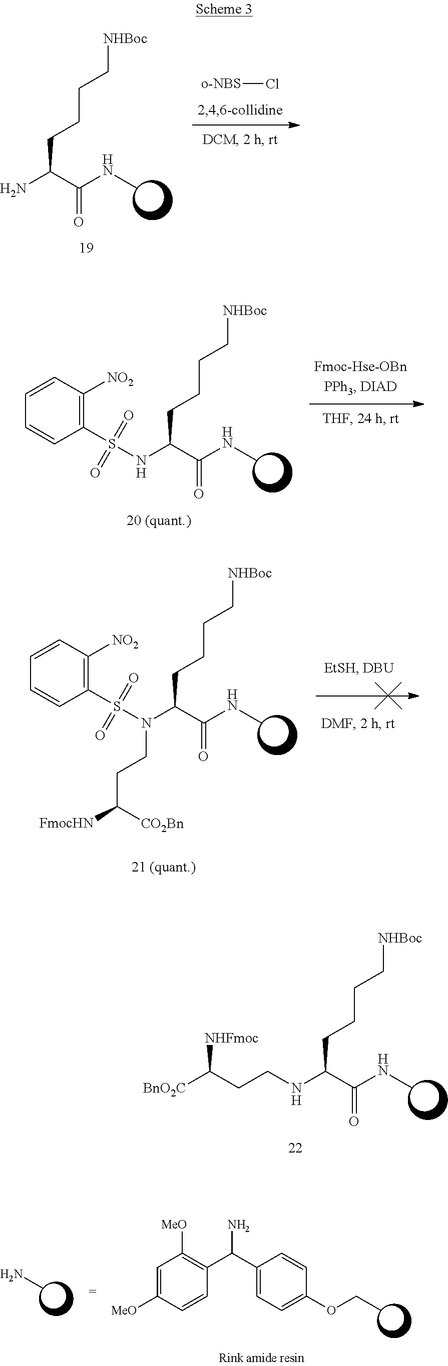

Peptide alkylation was pursued by silylation with N,O-bis(trimethylsilyl)acetamide (BSA), reaction with sulfamidates 8 and 17, and silyl group removal by washing the resin with methanol (Scheme 4). Resin-bound Lys(omega-Boc) 19 was thus swollen in dry THF, then flushed with argon, treated with BSA and shaken for 12 h (which caused an observable color change of the resin from yellow to lighter yellow), filtered under argon and treated with a solution of the appropriate sulfamidate (500 mol %) in dry THF. After 24 h, 17 and 8 gave respectively monoalkylated products in 64% and 97% conversion, with <2% bisalkylated product, as assessed by LC-MS analysis of material cleaved from resin samples.

Lactam formation was next performed by heating secondary amine resins 22a and 22b at 100° C. in DMF over 3 days (Scheme 4). This time was reduced to 3 h in the case of Bgl and 75 min in the case of Agl by microwave heating at 100° C. in DMF, conditions which gave cleaner products and >95% conversion in both cases.

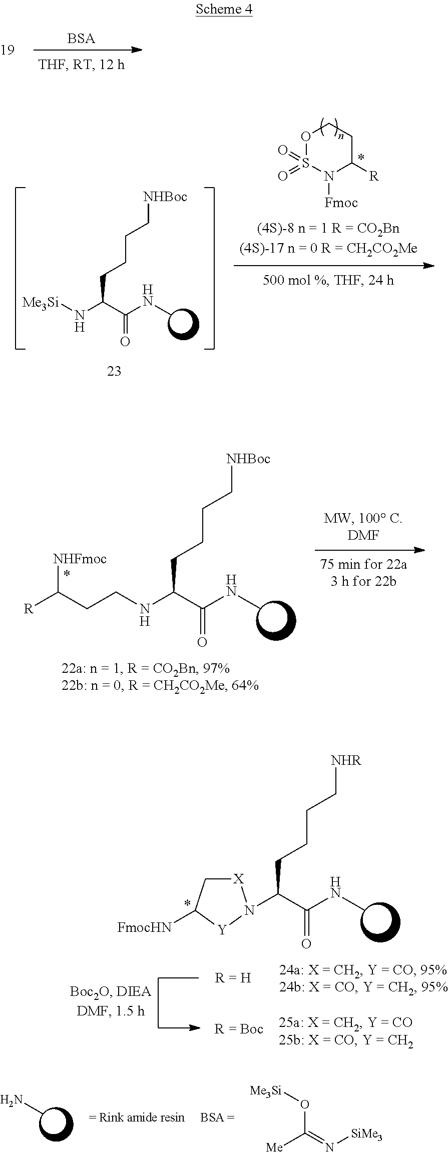

At the lactam forming step, the resulting resins 24a and 24b exhibited positive Kaiser tests,^{lix} indicating the presence of primary amines, which were considered to be due to loss of the Boc group from the Lys residue by thermolysis (Fmoc-Bgl/Agl-Lys-NH-Rink) as well as deprotected and unreacted starting peptide (Lys-NH-Rink). This was verified by treating an aliquot of resin 22b with Fmoc-OSu in the presence of DIEA in DCM. After TFA cleavage, analysis by LCMS indicated Fmoc-Bgl-Lys(Fmoc)-NH₂ as the major compound (60%) along with Fmoc-Lys(Fmoc)-NH₂ (30%). A capping step was therefore introduced after the lactam formation by treating the resin with di-tert-butyl dicarbonate (Boc₂O) and DIEA in DMF for 1.5 h, after which time resins 25a and 25b gave negative Kaiser tests (Scheme 4).

The synthesis of Agl tripeptides 34 and 35 was performed to assess configurational integrity during the synthesis, because RP-HPLC analysis of crude Agl peptides 44-48 and 54-59 (vide infra) showed small quantities (<10%) of an isobaric species. Rink resin bound L-N-(Fmoc)alanine 26 was deprotected, silylated and alkylated using either (S)- or (R)-sulfamidate 8 as described above (Scheme 4). Lactam annulation was performed by microwave heating at 110° C. in DMF over 3 h. Samples were cleaved and analyzed by RP-HPLC which indicated 5-10% of a second diastereoisomer. No further epimerization was observed after the amino lactam peptides were deprotected, coupled to L-Fmoc-Ala-OH, deprotected and cleaved from the resin. The source of the epimerization was later shown to be the homoserine by chiral Supercritical Fluid Chromatography (SFC) analysis of L-Fmoc-Hse-OBn (2S)-10b prepared in-house from L-Met and prepared from commercial homoserine (99% ee). The amount of epimer obtained from the synthesis of Agl tripeptides 34 and 35 corresponded with the purity of the starting homoserine, the preparation of which from methionine has previously been shown to cause racemization (Scheme 5).^{lia} Although enantiomerically pure sulfamidate 8 can be synthesized from commercially available homoserine, epimeric impurities were easily separated by preparative RP-HPLC from the desired products of lactam peptides made from sulfamidate 8 that was prepared in-house from methionine.

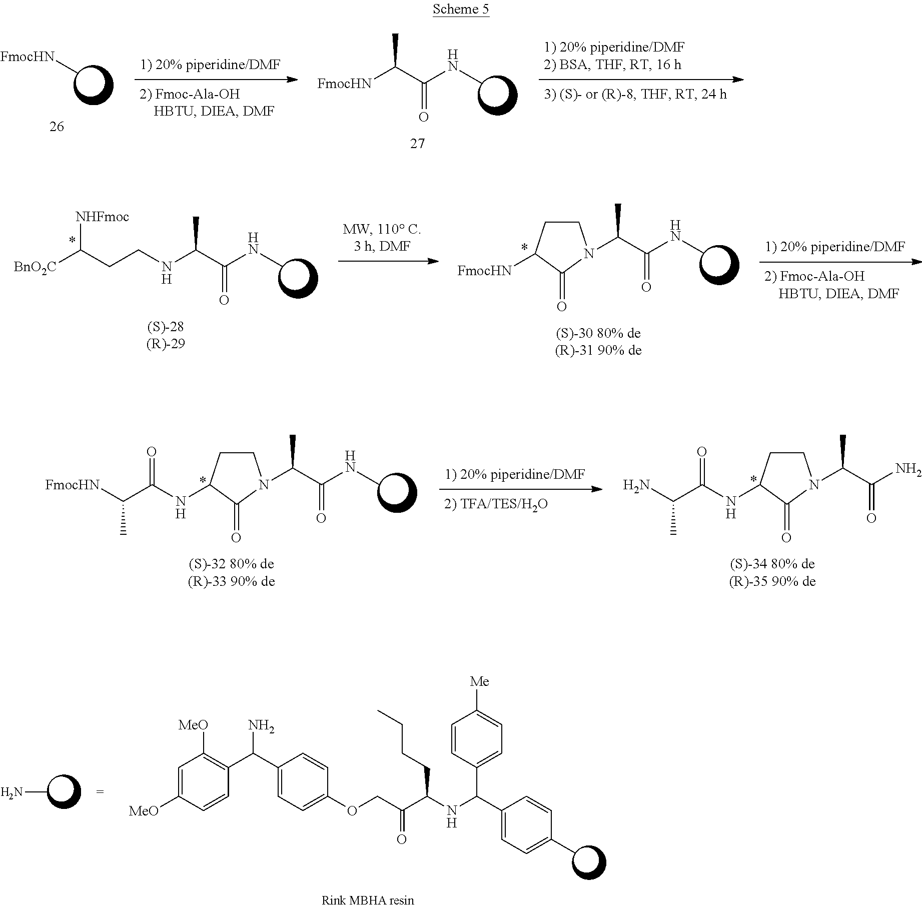

Only one epimer of crude Bgl peptides 49-53 (vide infra) could be detected by RP-HPLC under various conditions. The purity of sulfamidate 17 prepared from L- and D-aspartate was investigated by chiral SFC analysis, which showed sulfamidates (4S)- and (4R)-17 to have respectively 95.8% and 97.9% enantiomeric excess.

Ten analogues of growth hormone-releasing peptide-6 GHRP-6 were synthesized by using sulfamidates 8 and 17 in a general protocol for solid-phase lactam-bridged peptide synthesis (Scheme 6). Standard conditions for solid-phase peptide synthesis with Fmoc protection were first employed to synthesize the peptide chain 36 on Rink amide resin.^{lxiv} When the appropriate residue for lactam formation was reached, the N-terminal Fmoc group was removed and the resin was dried in vacuo. Using the above strategy, the N-terminal amine was silylated, alkylated with the respective sulfamidate (8 or 17), and converted to the lactam by microwave irradiation. After Fmoc removal, the remaining amino acid residues were coupled to the amino lactam residue 40 and 41 using standard coupling conditions. Reaction progress was monitored by LC-MS analyses of material cleaved from a small resin sample (3-5 mg) by treatment with a mixture of TFA/H₂O/TES (95:2.5:2.5, v/v/v, 1 mL) for 2 h, after filtration, evaporation and dissolution in acetonitrile.

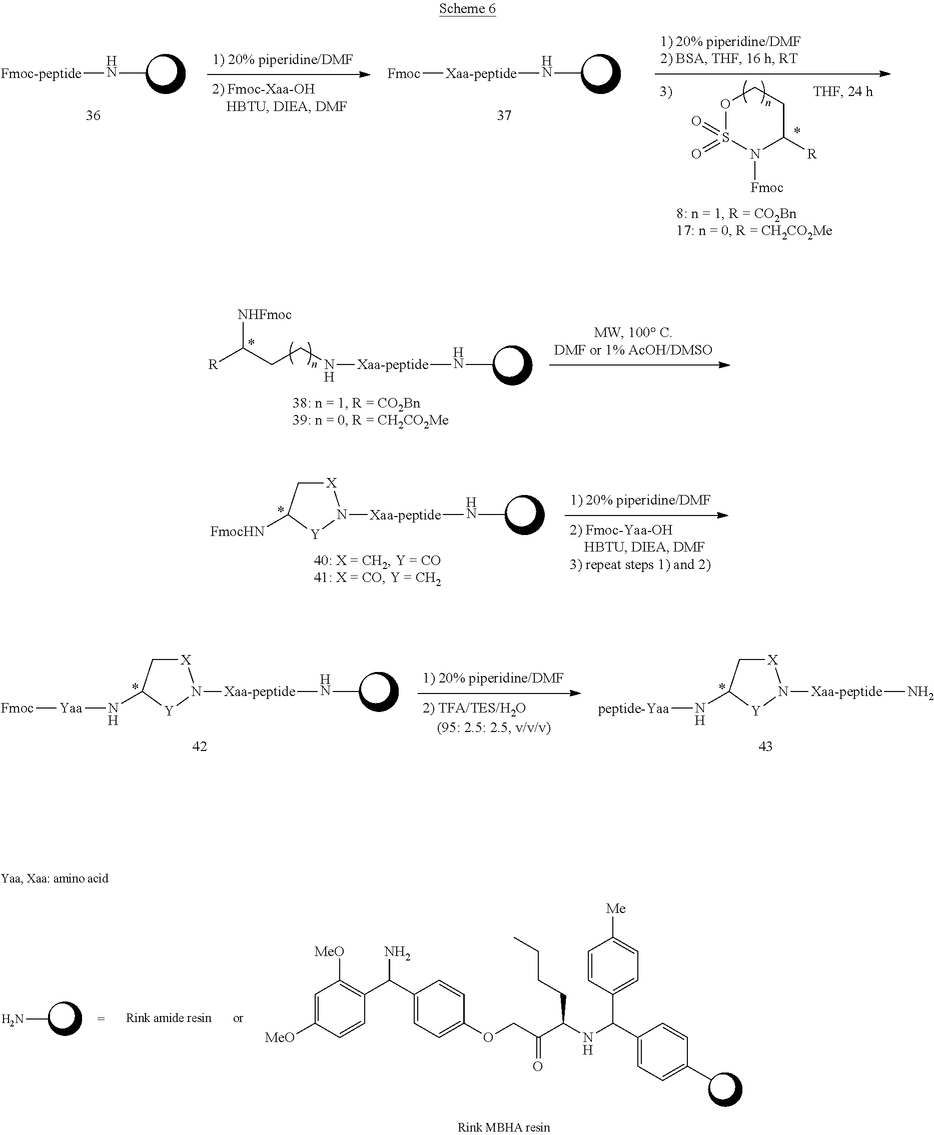

After solid-phase synthesis, the terminal Fmoc group of lactam peptide 42 was removed using 20% piperidine in DMF, and the peptide was cleaved by shaking in a mixture of TFA/TES/H₂O (95:2.5:2.5, v/v/v) for 2 h at room temperature. The crude lactam peptide 43 was precipitated in cold diethyl ether, analyzed by analytical RP-HPLC for crude purity and purified by preparative RP-HPLC to give the desired peptides 44-53 as white foams (Table 1).

Seeking analogues with greater potency and understanding of the conformation by which the D-heptapeptide 101.10 (rytvela) exhibits biological activity, a lactam scan was performed in which the robustness of the present methodology was further tested in the presence of sterically hindered β-branched residues [Val and Thr(O-^{t}Bu)]. Lactam peptides were made in higher yield and purity using Rink amide MBHA resin in the synthesis of 101.10 analogues, instead of Rink amide resin (Table 2). The solid-phase protocol was performed using (R)-cyclic sulfamidate (4R)-8 as described above for the preparation of GHRP-6 lactam analogs. The desired lactam peptides 54-59 were recovered in 36-81% crude purity as assessed by analytical RP-HPLC (Table 2). Exploring alternative microwave conditions, it was found that improved conversion to lactam was attained using 1% acetic acid in DMSO. Nevertheless, microwave assisted acylation required longer reaction times (>4 h) in the synthesis of analogs 55 and 56, presumably due to the difficulties of cyclisation on sterically bulky β-alkyl branched amino acids. For example, formation of Agl on the D-Thr(O-tBu) residue required 10 h of microwave heating of resin bound amine 38 at 110° C. in 1% acetic acid in DMSO and gave at best 50% conversion. Longer reaction times affected significantly the purity of the lactam peptides. The linear secondary amine could be isolated after the sequence of reactions by RP-HPLC and was subjected to lactam formation conditions in solution to provide additional lactam peptide. The main impurity observed by analytical RP-HPLC of the crude material after cleavage from resin was typically the deletion sequence from incomplete sulfamidate alkylation.

General Methods for Example 31

The general method used in this Example was the same as that for Examples 1-30 and in addition:

Rink amide resin SS (70-90 mesh, 0.7 mmol/g loading for synthesis in IRORI Kans™) was purchased from Advanced Chemtech™ (Louisville, Ky.). Analytical RP-HPLC analyses were preformed on a Gemini™ 5u C18 110A column (Phenomenex® Inc., Torrance, Calif., 150 mm×4.6 mm, 4 μm, column a) or a Synergi™ 4u Polar RP80A column (Phenomenex® Inc., Torrance, Calif., 150 mm×4.6 mm, 4 μm, column b) with a flow rate of 0.5 mL/min using a linear gradient of acetonitrile (0.1% formic acid (FA)) in water (0.1% FA). Retention times (t_{R}) from analytical RP-HPLC are reported in minutes. Otherwise specified, peptides were purified with a Gemini™5u C18 110A column (Phenomenex® Inc., Torrance, Calif., 250 mm×21.2 mm, 5 μm, column A, flow: 10.6 mL/min) or a Synergi™ 4u Polar RP80A Axial Packed column (Phenomenex® Inc., Torrance, Calif., 100 mm×21.2 mm, 4 μm, column B, flow: 7 mL/min) using a specified linear gradient acetonitrile (0.1% FA), in water (0.1% FA).

### Example 31

### Solid-Phase Peptide Synthesis Experimental Procedures in Syringe Tubes and IRORI Kan™ Solid-Phase Peptide Synthesis Experimental Procedures

Ten IRORI Kan™ macro-reactors were each charged with Rink amide resin SS (70-90 mesh, 300 mg, 0.65 mmol/g, 0.2 mmol) and a microchip. The kans were sealed and each kan identified with a serial number using the Synthesis Manager™ software. Peptide synthesis was then preformed as described above by placing the kans in a 250 mL glass bottle containing a stirrer bar. When washing the resin, residual solvent was removed from the kans by centrifugation. When the residue was reached for lactam synthesis, the appropriate kan was separated by scanning the microchips for the desired serial number. Fmoc deprotection, silylation and alkylation with either (R)-8 or (R)-17 was then performed on the single kan. After alkylation, the kan was opened and the resin transferred to a 5 mL glass microwave vessel and microwave assisted annulation preformed for the indicated time. The resin was then washed back into the IRORI Kan™ along with the micro-chip, the kan was sealed once again, returned to the other kans and peptide synthesis was completed.

### (a) GHRP-6 (R)-Agl Series

### (i) (R)-Agl-D-Trp-Ala-Trp-D-Phe-Lys-NH₂ (60)

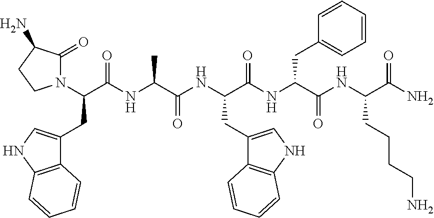

(R)-Agl-D-Trp-Ala-Trp-D-Phe-Lys-NH₂ 60 was prepared as described above in an IRORI Kan™, using microwave assisted annulation over 75 min, to give the desired lactam-bridged peptide TFA salt (97 mg) in 19% crude purity as determined by analytical RP-HPLC (column a, 2-40% MeCN in H₂O, 0.1% FA, 60 min gradient). Purification was carried out by preparative RP-HPLC (column A, 2-40% MeCN in H₂O, 0.1% FA, 60 min gradient) to give the desired formate salt (8 mg, 14%) as a white fluffy solid. The purified product was analyzed by analytical RP-HPLC (column a, UV: I=214 nm) using both MeCN t_{R} 11.39 (0-60% MeCN in H₂O, 0.1% FA, 30 min gradient) and MeOH t_{R} 14.50 (0-60% MeOH in H₂O, 0.1% FA, 30 min gradient) and revealed >93% purity. HRMS Calcd. m/z for C₄₄H₅₅O₆N₁₀ [M+H]⁺ 819.4301. found 819.4308.

### (ii) His-(R)-Agl-Ala-Trp-D-Phe-Lys-NH₂ (61)

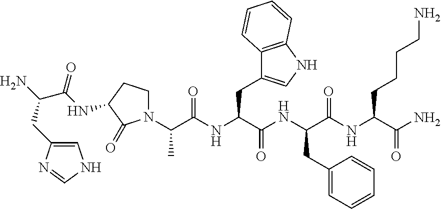

His-(R)-Agl-Ala-Trp-D-Phe-Lys-NH₂ 61 was prepared as described above in an IRORI Kan™, using microwave assisted annulation over 75 min, to give the desired lactam-bridged peptide TFA salt (178 mg) in 45% crude purity as determined by analytical RP-HPLC (column a, 0-20% MeCN in H₂O, 0.1% FA, 20 min gradient). Purification was carried out by preparative RP-HPLC (column A, 0-20% MeCN in H₂O, 0.1% FA, 25 min gradient) to give the desired formate salt (19 mg, 12%) as a white fluffy solid. The purified product was analyzed by analytical RP-HPLC (column a, UV: I=214 nm) using both MeCN t_{R} 8.80 (0-60% MeCN in H₂O, 0.1% FA, 30 min gradient) and MeOH t_{R} 10.03 (0-60% MeOH in H₂O, 0.1% FA, 30 min gradient) and revealed >99% purity. HRMS Calcd. m/z for C₃₉H₅₂O₆N₁₁ [M+H]⁺ 770.4096. found 770.4097.

### (iii) His-D-Trp-(R)-Agl-Trp-D-Phe-Lys-NH₂ (62)

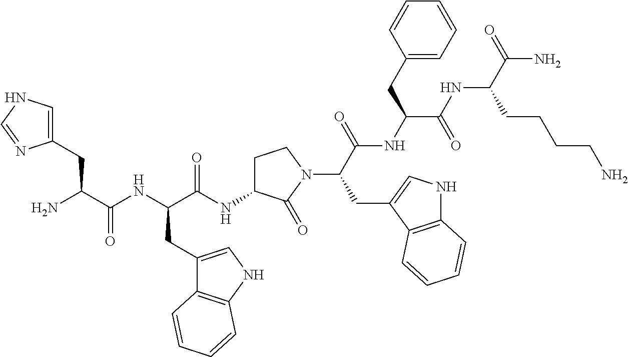

His-D-Trp-(R)-Agl-Trp-D-Phe-Lys-NH₂ 62 was prepared as described above in an IRORI Kan™, using microwave assisted annulation over 75 min, to give the desired lactam-bridged peptide TFA salt (144 mg) in 30% crude purity as determined by analytical RP-HPLC (column a, 0-40% MeCN in H₂O, 0.1% FA, 15 min gradient). Purification was carried out by preparative RP-HPLC (column A, 0-40% MeCN in H₂O, 0.1% FA, 60 min gradient) to give the desired formate salt product (7 mg, 4%) as a white fluffy solid. The purified product was analyzed by analytical RP-HPLC (column a, UV: I=214 nm) using both MeCN t_{R} 10.60 (0-60% MeCN in H₂O, 0.1% FA, 30 min gradient) and MeOH t_{R} 13.77 (0-60% MeOH in H₂O, 0.1% FA, 15 min gradient) and revealed >98% purity. HRMS Calcd. m/z for C₄₇H₅₈O₆N₁₂ [M+2H]²⁺ 443.2296. found 443.2301.

### (iv) His-D-Trp-Ala-(R)-Agl-D-Phe-Lys-NH₂ (63)

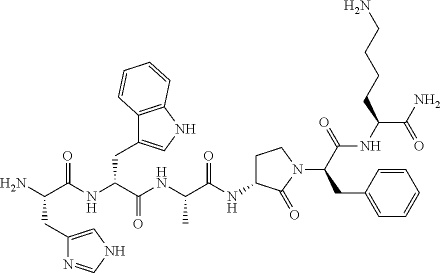

His-D-Trp-Ala-(R)-Agl-D-Phe-Lys-NH₂ 63 was prepared as described above in an IRORI Kan™, using microwave assisted annulation over 75 min, to give the desired lactam-bridged peptide TFA salt (132 mg) in 61% crude purity as determined by analytical RP-HPLC (column a, 0-20% MeCN in H₂O, 0.1% FA, 35 min gradient). Purification was carried out by preparative RP-HPLC (column A, 0-20% MeCN in H₂O, 0.1% FA, 20 min gradient) to give the desired formate salt product (17 mg, 11%) as a white fluffy solid. The purified product was analyzed by analytical RP-HPLC (column a, UV: I=214 nm) using both MeCN t_{R} 8.68 (0-60% MeCN in H₂O, 0.1% FA, 30 min gradient) and MeOH t_{R} 9.77 (0-60% MeOH in H₂O, 0.1% FA, 30 min gradient) and revealed >99% purity. HRMS Calcd. m/z for C₃₉H₅₂O₆N₁₁ [M+H]⁺ 770.4096. found 770.4097.

### (v)His-D-Trp-Ala-Trp-(R)-Agl-Lys-NH₂ (64)

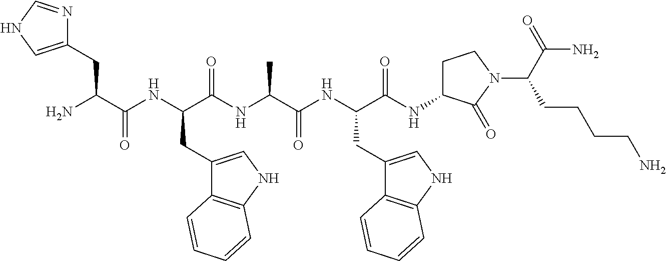

His-D-Trp-Ala-Trp-(R)-Agl-Lys-NH₂ 64 was prepared in as described above in an IRORI Kan™, using microwave assisted annulation over 90 min, to give the desired lactam-bridged peptide TFA salt (101 mg) in 55% crude purity as determined by analytical RP-HPLC (column a, 0-20% MeCN in H₂O, 0.1% FA, 15 min gradient). Purification was carried out by preparative RP-HPLC (column A, 0-20% MeCN in H₂O, 0.1% FA, 20 min gradient) to give the desired formate salt (11 mg, 7%) as a white fluffy solid. The purified product was analyzed by analytical RP-HPLC (column a, UV: I=214 nm) using both MeCN t_{R} 8.83 (0-60% MeCN in H₂O, 0.1% FA, 30 min gradient) and MeOH t_{R} 10.21 (0-60% MeOH in H₂O, 0.1% FA, 30 min gradient) and revealed >99% purity. HRMS Calcd. m/z for C₄₁H₅₃O₆N₁₂ [M+H]⁺ 809.4205. found 809.4199.

### (b) GHRP-6 (R)-Bgl Series

### (i) (R)-Bgl-D-Trp-Ala-Trp-D-Phe-Lys-NH₂ (65)

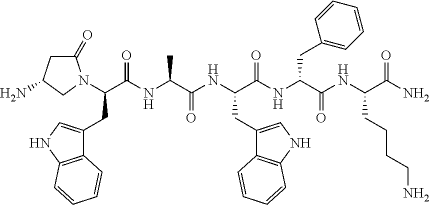

(R)-Bgl-D-Trp-Ala-Trp-D-Phe-Lys-NH₂ 65 was prepared in an IRORI Kan™ as described above, using microwave assisted annulation over 180 min to give the desired lactam peptide TFA salt 65 (135 mg) in 30% crude purity as determined by analytical RP-HPLC (column a, UV: I=214 nm, 0-30% MeCN in H₂O, 0.1% FA, 20 min gradient). Purification was carried out by preparative RP-HPLC (column A, 10-25% MeCN in H₂O, 0.1% FA, 50 min gradient, flow rate: 15 mL/min) to give the desired formate salt 65 (9.6 mg, 5%) as a white fluffy solid. The purified product was analyzed by analytical RP-HPLC (column a, UV: I=214 nm) using both MeCN t_{R} 15.50 (0-60% MeCN in H₂O, 0.1% FA, 25 min gradient) and MeOH t_{R} 13.50 (0-60% MeOH in H₂O, 0.1% FA, 25 min gradient) and revealed >97% purity. HRMS Calcd. m/z for C₄₄H₅₅O₆N₁₀ [M+H]⁺ 819.4301. found 819.4307.

### (ii) Ac(R)-Bgl-D-Trp-Ala-Trp-D-Phe-Lys-NH₂ (66)

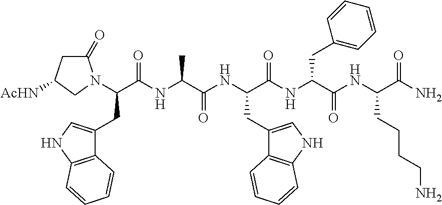

H₂N—(R)-Bgl-D-Trp(Boc)-Ala-Trp(Boc)-D-Phe-Lys(Boc)-NH-Rink 66 was prepared in an IRORI Kan™ as described above, using microwave assisted annulation over 180 min. It was acetylated using a solution of acetic anhydride (5 eq.) and DIEA (5 eq.) in DMF (4 mL) over 1.5 h. After washing, cleavage and precipitation as described above, the desired lactam peptide TFA salt 66 (135 mg) was obtained in 20% crude purity as determined by analytical RP-HPLC (column a, UV: I=214 nm, 0-30% MeCN in H₂O, 0.1% FA, 20 min gradient). Purification was carried out by preparative RP-HPLC (column A, 20-40% MeCN in H₂O, 0.1% FA, 40 min gradient, flow rate: 15 mL/min) to give the desired formate salt 66 (14 mg, 7.4%) as a white fluffy solid. The purified product was analyzed by analytical RP-HPLC (column a, UV: I=214 nm) using both MeCN t_{R} 14.42 (0-80% MeCN in H₂O, 0.1% FA, 25 min gradient) and MeOH t_{R} 19.42 (0-90% MeOH in H₂O, 0.1% FA, 25 min gradient) and revealed >99% purity. HRMS Calcd. m/z for C₄₆H₅₇O₇N₁₀ [M+H]⁺ 861,4406. found 861,4413.

### (iii) His-(R)-Bgl-Ala-Trp-D-Phe-Lys-NH₂ (67)

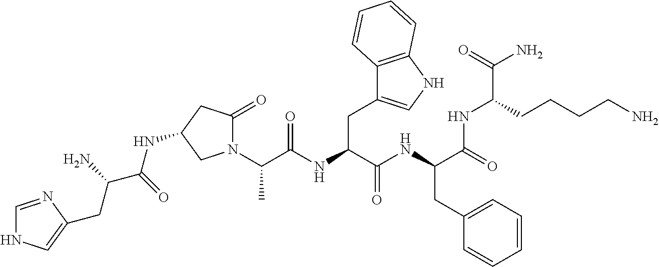

His-(R)-Bgl-Ala-Trp-D-Phe-Lys-NH₂ 67 was prepared in a syringe tube following the optimized protocol as described above, using microwave assisted annulation over 180 min, to give the desired lactam peptide TFA salt 67 (112 mg) in 31% crude purity as determined by analytical RP-HPLC (column b, UV: I=214 nm, 0-80% MeCN in H₂O, 0.1% FA, 30 min gradient). Purification was carried out by preparative RP-HPLC (column B, 10-40% MeCN in H₂O, 0.1% FA, 30 min gradient) to give the desired formate salt product 67 (16 mg, 7.8%) as a white fluffy solid. The purified product was analyzed by analytical RP-HPLC (column b, UV: I=214 nm) using both MeCN t_{R} 14.26 (0-60% MeCN in H₂O, 0.1% FA, 25 min gradient) and MeOH t_{R} 15.91 (0-60% MeOH in H₂O, 0.1% FA, 30 min gradient) and revealed >99% purity. HRMS Calcd. m/z for C₃₉H₅₃O₆N₁₁ [M+2H]²⁺ 385.7085. found 385.7083.

### (iv) His-D-Trp-(R)-Bgl-Trp-D-Phe-Lys-NH₂ (68)

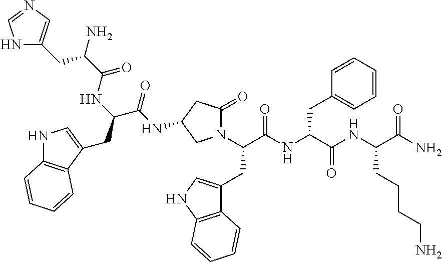

His-D-Trp-(R)-Bgl-Trp-D-Phe-Lys-NH₂ 68 was prepared in a syringe tube following the optimized protocol as described above, using microwave assisted annulation over 180 min to give the desired lactam peptide TFA salt 68 (120 mg) in 22% crude purity as determined by analytical RP-HPLC (column b, UV: I=214 nm, 15-40% MeCN in H₂O, 0.1% FA, 30 min gradient). Purification was carried out by preparative RP-HPLC (column B, 8-30% MeCN in H₂O, 0.1% FA, 30 min gradient) to give the desired formate salt 68 (14 mg, 7.9%) as a white fluffy solid. The purified product was analyzed by analytical RP-HPLC (column a, UV: I=214 nm) using both MeCN t_{R} 17.57 (0-60% MeCN in H₂O, 0.1% FA, 25 min gradient) and MeOH t_{R} 21.38 (0-80% MeOH in H₂O, 0.1% FA, 25 min gradient) and revealed >99% purity. HRMS Calcd. m/z for C₄₇H₅₈O₆N₁₂ [M+2H]²⁺ 443.2296. found 443.2301.

### (v) His-D-Trp-Ala-(R)-Bgl-D-Phe-Lys-NH₂ (69)

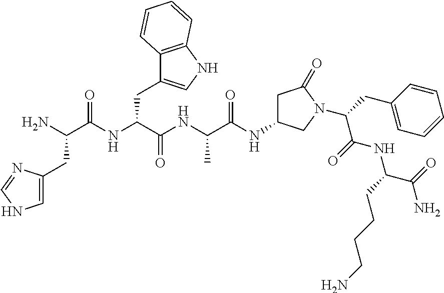

His-D-Trp-Ala-(R)-Bgl-D-Phe-Lys-NH₂ 69 was prepared in an IRORI Kan™ was prepared as described above, using microwave assisted annulation over 180 min to give the desired lactam peptide TFA salt 69 (180 mg) in 40% crude purity as determined by analytical RP-HPLC (column a, UV: I=214 nm, 0-30% MeCN in H₂O, 0.1% FA, 25 min gradient). Purification was carried out by preparative RP-HPLC (column A, 10-25% MeCN in H₂O, 0.1% FA, 20 min gradient) to give the desired formate salt product 69 (9.6 mg, 5%) as a white fluffy solid. The purified product was analyzed by analytical RP-HPLC (column a, UV: I=214 nm) using both MeCN t_{R} 12.13 (0-60% MeCN in H₂O, 0.1% FA, 20 min gradient) and MeOH t_{R} 14.32 (0-60% MeOH in H₂O, 0.1% FA, 20 min gradient) and revealed >99% purity. HRMS Calcd. m/z for C₃₉H₅₂O₆N₁₁ [M+H]⁺ 770.4096. found 770.4095.

### (vi) His-D-Trp-Ala-Trp-(R)-Bgl-Lys-NH₂ (70)

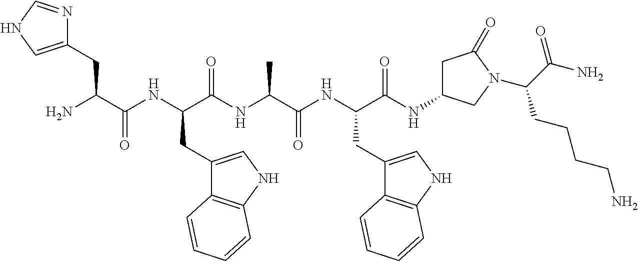

His-D-Trp-Ala-Trp-(R)-Bgl-Lys-NH₂ 70 was prepared in an IRORI Kan™ as described above, using microwave assisted annulation over 180 min, to give the desired lactam peptide TFA salt 70 (67 mg) in 60% crude purity as determined by analytical RP-HPLC (column a, UV: I=214 nm, 0-30% MeCN in H₂O, 0.1% FA, 25 min gradient). Purification was carried out by preparative RP-HPLC (column A, 0-30% MeCN in H₂O, 0.1% FA, 25 min gradient) to give the desired formate salt 70 (5.4 mg, 3%) as a white fluffy solid. The purified product was analyzed by analytical RP-HPLC (column a, UV: I=214 nm) using both MeCN t_{R} 12.05 (0-60% MeCN in H₂O, 0.1% FA, 20 min gradient) and MeOH t_{R} 14.11 (0-60% MeOH in H₂O, 0.1% FA, 20 min gradient) and revealed >99% purity. HRMS Calcd. m/z for C₄₁H₅₃O₆N₁₂ [M+H]⁺ 809.4205. found 809.4198.

Discussion for Example 31

With the goal to accelerate the process of lactam scanning, IRORI Kan™ macro-reactors (300 mg resin capacity)^{lxv} were utilized in a combinatorial synthesis strategy to perform a (R)-amino lactam scan of GHRP-6 using 6-membered cyclic sulfamidate 8, and 5-membered cyclic sulfamidate 17.

Ten IRORI Kans™, each containing a microchip with designated serial numbers and 300 mg of Rink amide resin (70-90 mesh, 0.7 mmol/g), were employed in parallel peptide synthesis in a 200 mL glass batch reaction vessel. At the lactam formation step, the appropriate IRORI Kan™, identified by scanning the microchip serial number, was removed from the batch reaction vessel and the silylation and alkylation steps were performed in a 5 mL glass microwave reaction vessel, after the IRORI Kan™ microchip was temporarily removed from the IRORI Kan™. Microwave assisted annulation was performed at 100° C. in 1% acetic acid/DMSO for the specified time. The resin was then washed back into the IRORI Kan™ along with the microchip, and the Kan™ was recombined with the others and peptide synthesis continued to completion. The terminal Fmoc group was removed using 20% piperidine in DMF. The IRORI Kans™ were then separated, identified by microchip serial number, placed inside polystyrene syringe tubes and the crude peptides were cleaved off the resin, analysed for purity and purified as described before to give the desired peptides 60-71 as white fluffy solids (Table 3).

Using a standard protocol, lactam peptides 60-66 and 69-70 were prepared in 19-61% crude purity and isolated in acceptable yields after purification. The syntheses of peptides 67 and 68 were, however, problematic, giving low crude purity (<10%), both in IRORI Kans™ and syringe tubes. Although alkylation and lactam formation appeared to go with acceptable conversion, the standard method was retooled for these difficult sequences for lactam synthesis.

In the optimized protocol, better crude purities, yields and ease of purification were obtained by eliminating the silylation step, and performing the alkylation reaction at 60° C. under microwave irradiation for 1 h., in the presence of 5 eq. of cyclic sulfamidate 17 and 1 eq. of DIEA. Using these conditions for making Bgl peptide, no noticeable bis-alkylation product was measured by LC-MS analysis of material cleaved after the alkylation step. Employing more than 1.1 eq. of DIEA as base caused formation of detectable amounts of bis alkylation product. In the alkylation step, ACS grade THF worked as effectively as dry THF. Improvements in lactam formation yields were obtained by washing the resin beforehand with 2% AcOH in DMSO for 30 min followed by MeCN for 5 min, and microwave irradiation at 100° C. for three hours in a DMSO:H₂O:AcOH 75:23:2 mixture. These modifications of our standard protocol gave peptides 67 and 68 respectively in 35% and 21% crude purity; both being isolated in 8% yield after purification on preparative RP-HPLC (Scheme 7)

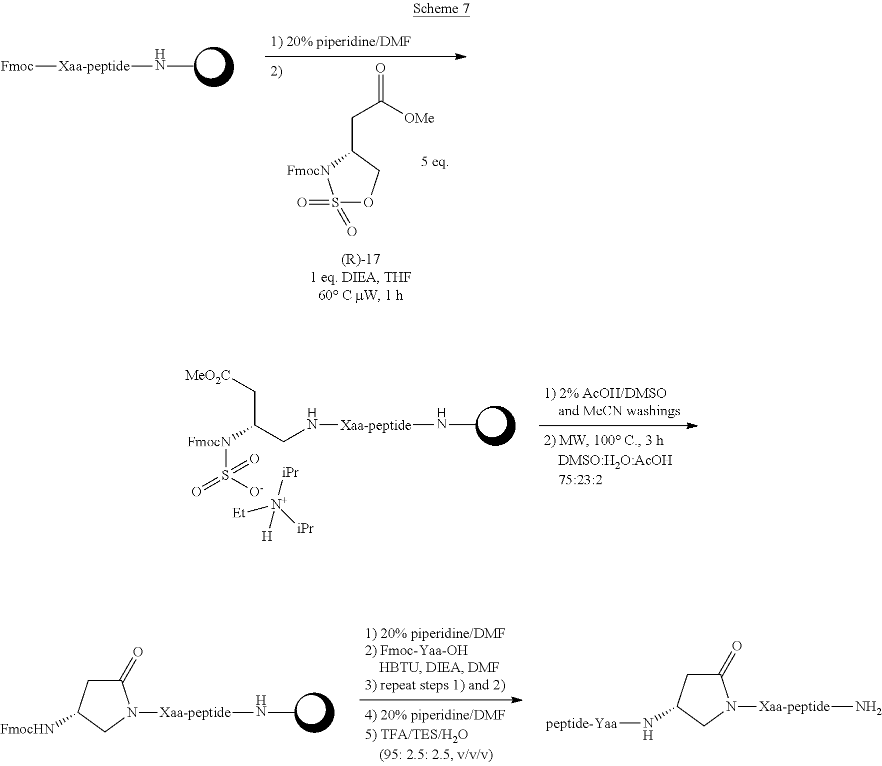

### Example 32

### Optimized Protocol for (R)-Bgl Lactam Peptide Synthesis

(S)- and (R)-Bgl 101.10 analogs were synthesized using optimized conditions. Peptide synthesis was carried out in syringe tubes as described earlier. When the residue was reached for lactam synthesis, Fmoc deprotection was performed as usual and the resin was transferred into a 5 mL glass microwave vessel. A solution of cyclic sulfamidate (R)-17 (5 eq.) and DIEA (1 eq.) in THF (2 mL) was added, the reactor was sealed and the mixture was irradiated under microwave at 60° C. for 1 h. The resin was then transferred into a 6 mL syringe tube, washed with DMF (3 times), MeOH (3 times), DCM (3 times), and with three cycles comprising washing with 2% AcOH in DMSO for 30 min and washing with MeCN for 5 minutes. The resin was transferred back to a 5 mL glass microwave vessel, a DMSO:H₂O:AcOH 75:23:2 (2 mL) was added, the reactor sealed and the mixture was irradiated under microwave at 100° C. for 3 h. The resin was transferred back into a syringe tube and underwent an acetyl-capping step by treatment with a solution of acetic anhydride (5 eq.) in DMF (4 mL) in the presence of DIEA (5 eq.) for 1.5 h. The peptide synthesis was then continued as previously described.

### (R)-Bab 101.10 Analogs

### Example 33

### (R)-Bab-D-Tyr-D-Thr-D-Val-D-Glu-D-Leu-D-Ala-NH₂ 71

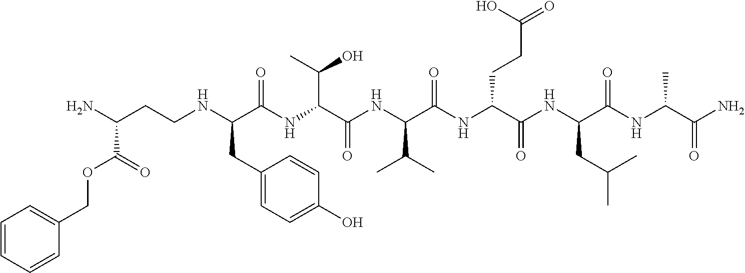

(R)-Bab-D-Tyr-D-Thr-D-Val-D-Glu-D-Leu-D-Ala-NH₂ 71 was isolated while purifying compound 54, prepared as described above (28% of 54 crude mixture as analyzed by analytical RP-HPLC (column a, UV: I=214 nm, 2-40% MeCN in H₂O, 0.1% FA, 15 min gradient). Purification was carried out by preparatory RP-HPLC (column A, 2-20% MeCN in H₂O, 0.1% FA, 20 min gradient) to give the desired formic acid salt 71 (10 mg, 10%) as a white fluffy solid. The purified product was analyzed by analytical RP-HPLC (column a, UV: I=214 nm) using both MeCN t_{R} 14.11 (5-40% MeCN in H₂O, 0.1% FA, 15 min gradient) and MeOH t_{R} 12.69 (20-80% MeOH in H₂O, 0.1% FA, 15 min) and revealed >99% purity. HRMS Calcd. for C₄₃H₆₄O₁₂N₈ [M+H]⁺ 885.4714. found 885.4716.

### Example 34

### D-Arg-(R)-Bab-D-Thr-D-Val-D-Glu-D-Leu-D-Ala-NH₂ 72

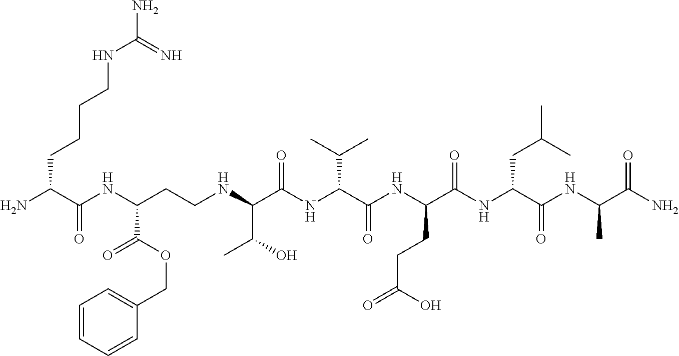

D-Arg-(R)-Bab-D-Thr-D-Val-D-Glu-D-Leu-D-Ala-NH₂ 72 was isolated while purifying compound 55, prepared as described above (45% of 55 crude mixture as analyzed by analytical RP-HPLC (column a, UV: I=214 nm, 0-20% MeCN in H₂O, 0.1% FA, 15 min gradient). Purification was carried out by preparatory RP-HPLC (column A, 0-20% MeCN in H₂O, 0.1% FA, 30 min gradient) to give the desired formic acid salt 72 (27 mg, 15%) as a white fluffy solid. The purified product was analyzed by analytical RP-HPLC (column a, UV: I=214 nm) using both MeCN t_{R} 9.04 (0-60% MeCN in H₂O, 0.1% FA, 30 min gradient) and MeOH t_{R} 11.00 (0-60% MeOH in H₂O, 0.1% FA, 30 min) and revealed >99% purity. HRMS Calcd. for C₄₀H₆₈O₁₁N₁₁ [M+H]⁺ 878.5092. found 878.5094.

### Example 35

### D-Arg-D-Tyr-(R)-Bab-D-Val-D-Glu-D-Leu-D-Ala-NH₂ 73

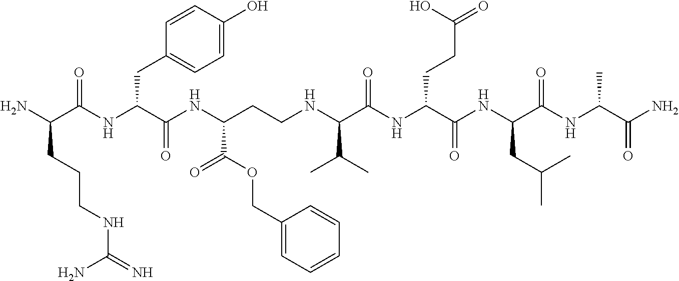

D-Arg-D-Tyr-D-Thr-(R)-Bab-D-Glu-D-Leu-D-Ala-NH₂ 73 was isolated while purifying compound 56, prepared as described above (27% of 56 crude mixture as analyzed by analytical RP-HPLC (column a, UV: I=214 nm, 0-40% MeCN in H₂O, 0.1% FA, 15 min gradient). Purification was carried out by preparatory RP-HPLC (column A, 0-40% MeCN in H₂O, 0.1% FA, 30 min gradient) to give the desired formic acid salt 73 (7 mg, 3%) as a white fluffy solid. The purified product was analyzed by analytical RP-HPLC (column a, UV: I=214 nm) using both MeCN t_{R} 9.12 (0-60% MeCN in H₂O, 0.1% FA, 15 min gradient) and MeOH t_{R} 9.45 (0-60% MeOH in H₂O, 0.1% FA, 15 min) and revealed >99% purity. HRMS Calcd. for C₄₅H₇₀O₁₁N₁₁ [M+H]⁺ 940.5240. found 940.5251.

### Example 36

### D-Arg-D-Tyr-D-Thr-(R)-Bab-D-Glu-D-Leu-D-Ala-NH₂ 74

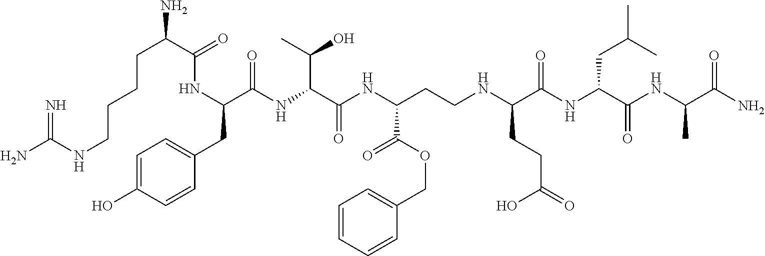

D-Arg-D-Tyr-D-Thr-(R)-Bab-D-Glu-D-Leu-D-Ala-NH₂ 74 was isolated while purifying compound 57, prepared as described above (6% of 57 crude mixture as analyzed by analytical RP-HPLC (column a, UV: I=214 nm, 2-40% MeCN in H₂O, 0.1% FA, 15 min gradient). Purification was carried out by preparatory RP-HPLC (column A, 2-20% MeCN in H₂O, 0.1% FA, 20 min gradient) to give the desired formic acid salt 74 (8.5 mg, 4%) as a white fluffy solid. The purified product was analyzed by analytical RP-HPLC (column a, UV: I=214 nm) using both MeCN t_{R} 12.27 (5-40% MeCN in H₂O, 0.1% FA, 15 min gradient) and MeOH t_{R} 17.09 (5-40% MeOH in H₂O, 0.1% FA, 15 min) and revealed >99% purity. HRMS Calcd. for C₄₄H₆₈O₁₂N₁₁ [M+H]⁺ 942.5027. found 942.5043.

### (R)-Bgl 101.10 Scan Compounds

### Example 37

### (R)-Bgl-D-Tyr-D-Thr-D-Val-D-Glu-D-Leu-D-Ala-NH₂ 75

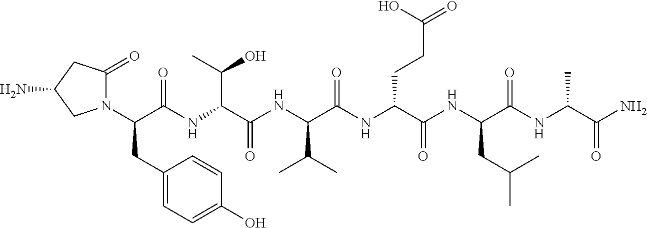

(R)-Bgl-D-Tyr-D-Thr-D-Val-D-Glu-D-Leu-D-Ala-NH₂ 75 was prepared on a 0.072 mmol, in a syringe tube following the optimized protocol as described above, using microwave assisted annulation over 240 min, to give the desired lactam peptide TFA salt 75 (34 mg) in 71% crude purity as determined by analytical RP-HPLC (column b, UV: I=214 nm, 0-80% MeCN in H₂O, 0.1% FA, 30 min gradient). Purification was carried out by preparative RP-HPLC (column B, 10-30% MeCN in H₂O, 0.1% FA, 30 min gradient) to give the desired FA salt product 75 (13 mg, 22%) as a white fluffy solid. The purified product was analyzed by analytical RP-HPLC (column b, UV: I=214 nm) using both MeCN t_{R} 11.8 (5-60% MeCN in H₂O, 0.1% FA, 25 min gradient) and MeOH t_{R} 16.41 (0-80% MeOH in H₂O, 0.1% FA, 25 min gradient) and revealed >99% purity. HRMS Calcd. m/z for C₃₆H₅₇O₁₁N₈ [M+H]⁺ 777.4141. found 777.4145.

### Example 38

### D-Arg-(R)-Bgl-D-Thr-D-Val-D-Glu-D-Leu-D-Ala-NH₂ 76

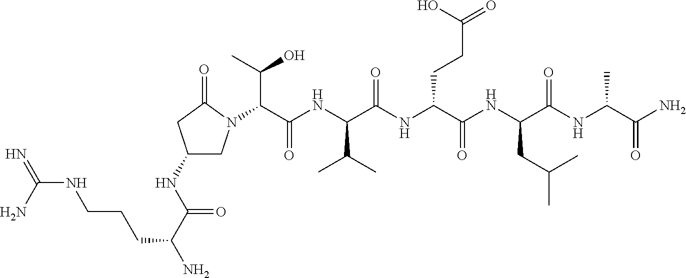

D-Arg-(R)-Bgl-D-Thr-D-Val-D-Glu-D-Leu-D-Ala-NH₂ 76 was prepared on a 0.078 mmol scale, in a syringe tube following the optimized protocol as described above, using microwave assisted annulation over 360 min, to give the desired lactam peptide TFA salt 76 (35 mg) in 87% crude purity as determined by analytical RP-HPLC (column a, UV: I=214 nm, 0-20% MeCN in H₂O, 0.1% FA, 30 min gradient). Purification was carried out by preparative RP-HPLC (column A, 2-20% MeCN in H₂O, 0.1% FA, 30 min gradient) to give the desired FA salt product 76 (16 mg, 24%) as a white fluffy solid. The purified product was analyzed by analytical RP-HPLC (column a, UV: I=214 nm) using both MeCN t_{R} 7.79 (5-60% MeCN in H₂O, 0.1% FA, 25 min gradient) and MeOH t_{R} 10.28 (0-80% MeOH in H₂O, 0.1% FA, 25 min gradient) and revealed >99% purity. HRMS Calcd. m/z for C₃₃H₆₀O₁₀N₁₁ [M+H]⁺ 770.4519. found 770.4521.

### Example 39

### D-Arg-D-Tyr-(R)-Bgl-D-Val-D-Glu-D-Leu-D-Ala-NH₂ 77

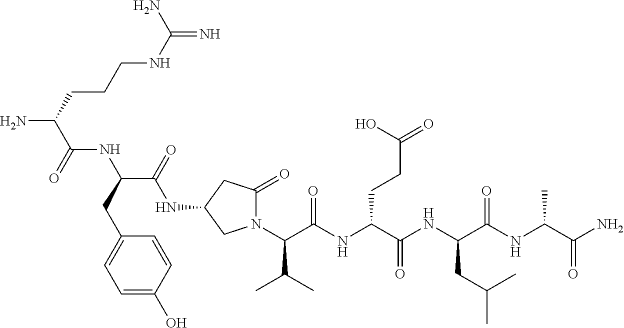

D-Arg-D-Tyr-(R)-Bgl-D-Val-D-Glu-D-Leu-D-Ala-NH₂ 77 was prepared on a 0.082 mmol scale, in a syringe tube following the optimized protocol as described above, using microwave assisted annulation over 240 min, to give the desired lactam peptide TFA salt 77 (34 mg) in 86% crude purity as determined by analytical RP-HPLC (column b, UV: I=214 nm, 0-20% MeCN in H₂O, 0.1% FA, 30 min gradient). Purification was carried out by preparative RP-HPLC (column B, 0-20% MeCN in H₂O, 0.1% FA, 30 min gradient) to give the desired FA salt product 77 (17 mg, 23%) as a white fluffy solid. The purified product was analyzed by analytical RP-HPLC (column b, UV: I=214 nm) using both MeCN t_{R} 9.55 (5-60% MeCN in H₂O, 0.1% FA, 25 min gradient) and MeOH t_{R} 12.99 (0-80% MeOH in H₂O, 0.1% FA, 25 min gradient) and revealed >99% purity. HRMS Calcd. m/z for C₃₈H₆₂O₁₀N₁₁ [M+H]⁺ 832.4676. found 832.4677.

### Example 40

### D-Arg-D-Tyr-D-Thr-(R)-Bgl-D-Glu-D-Leu-D-Ala-NH₂ 78

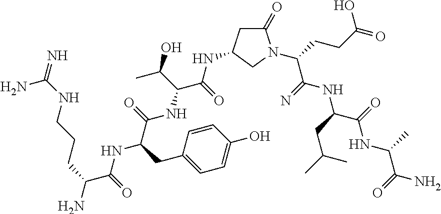

D-Arg-D-Tyr-D-Thr-(R)-Bgl-D-Glu-D-Leu-D-Ala-NH₂ 78 was prepared on a 0.087 mmol scale, in a syringe tube following the optimized protocol as described above, using microwave assisted annulation over 420 min, to give the desired lactam peptide TFA salt 78 (62 mg) in 79% crude purity as determined by analytical RP-HPLC (column b, UV: I=214 nm, 0-30% MeCN in H₂O, 0.1% FA, 30 min gradient). Purification was carried out by preparative RP-HPLC (column B, 18% MeCN in H₂O, 0.1% FA, isocratic) to give the desired FA salt product 78 (16 mg, 17%) as a white fluffy solid. The purified product was analyzed by analytical RP-HPLC (column b, UV: I=214 nm) using both MeCN t_{R} 8.00 (5-60% MeCN in H₂O, 0.1% FA, 25 min gradient) and MeOH t_{R} 9.93 (0-80% MeOH in H₂O, 0.1% FA, 25 min gradient) and revealed >99% purity. HRMS Calcd. m/z for C₃₇H₆₀O₁₁N₁₁ [M+H]⁺ 834.4468. found 834.4470.

### Example 41

### D-Arg-D-Tyr-D-Thr-D-Val-(R)-Bgl-D-Leu-D-Ala-NH₂ 79

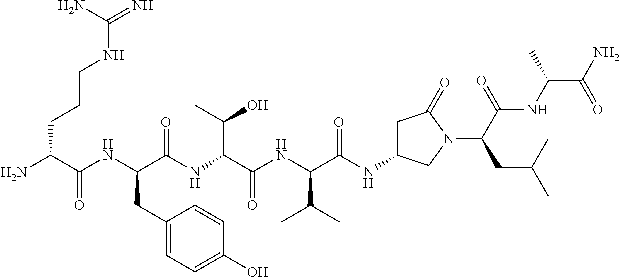

D-Arg-D-Tyr-D-Thr-D-Val-(R)-Bgl-D-Leu-D-Ala-NH₂ 79 was prepared on a 0.098 mmol scale, in a syringe tube following the optimized protocol as described above, using microwave assisted annulation over 240 min, to give the desired lactam peptide TFA salt 79 (35 mg) in 87% crude purity as determined by analytical RP-HPLC (column a, UV: I=214 nm, 0-80% MeCN in H₂O, 0.1% FA, 30 min gradient). Purification was carried out by preparative RP-HPLC (column A, 0-30% MeCN in H₂O, 0.1% FA, 30 min gradient) to give the desired FA salt product 79 (6 mg, 7%) as a white fluffy solid. The purified product was analyzed by analytical RP-HPLC (column a, UV: I=214 nm) using both MeCN t_{R} 9.67 (5-60% MeCN in H₂O, 0.1% FA, 25 min gradient) and MeOH t_{R} 13.18 (0-80% MeOH in H₂O, 0.1% FA, 25 min gradient) and revealed >99% purity. HRMS Calcd. m/z for C₃₇H₆₁O₉N₁₁ [M+Na]⁺ 826.4546. found 826.4544.

### Example 42

### D-Arg-D-Tyr-D-Thr-D-Val-D-Glu-(R)-Bgl-D-Ala-NH₂ 80

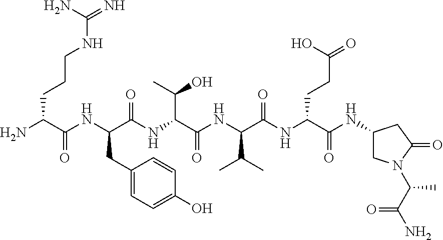

D-Arg-D-Tyr-D-Thr-D-Val-D-Glu-(R)-Bgl-D-Ala-NH₂ 80 was prepared on a 0.105 mmol scale, in a syringe tube following the optimized protocol as described above, using microwave assisted annulation over 240 min, to give the desired lactam peptide TFA salt 80 (35 mg) in 50% crude purity as determined by analytical RP-HPLC (column b, UV: I=214 nm, 0-60% MeCN in H₂O, 0.1% FA, 25 min gradient). Purification was carried out by preparative RP-HPLC (column B, 0-20% MeCN in H₂O, 0.1% FA, 30 min gradient) to give the desired FA salt product 80 (13 mg, 14%) as a white fluffy solid. The purified product was analyzed by analytical RP-HPLC (column b, UV: I=214 nm) using both MeCN t_{R} 7.27 (5-60% MeCN in H₂O, 0.1% FA, 25 min gradient) and MeOH t_{R} 9.04 (0-80% MeOH in H₂O, 0.1% FA, 25 min gradient) and revealed >99% purity. HRMS Calcd. m/z for C₃₆H₅₈O₁₁N₁₁ [M+H]⁺ 820.4312. found 820.4313.

### (S)-Bgl 101.10 Scan Compounds

### Example 43

### (S)-Bgl-D-Tyr-D-Thr-D-Val-D-Glu-D-Leu-D-Ala-NH₂ 81

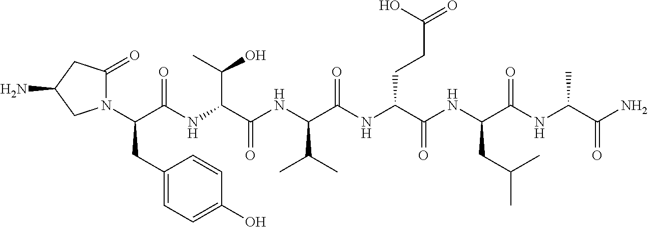

(S)-Bgl-D-Tyr-D-Thr-D-Val-D-Glu-D-Leu-D-Ala-NH₂ 81 was prepared on a 0.102 mmol scale, in a syringe tube following the optimized protocol as described above, using microwave assisted annulation over 600 min, to give the desired lactam peptide TFA salt 81 (34 mg) in 70% crude purity as determined by analytical RP-HPLC (column a, UV: I=214 nm, 0-80% MeCN in H₂O, 0.1% FA, 30 min gradient). Purification was carried out by preparative RP-HPLC (column A, 10-30% MeCN in H₂O, 0.1% FA, 30 min gradient) to give the desired FA salt product 81 (11 mg, 13%) as a white fluffy solid. The purified product was analyzed by analytical RP-HPLC (column a, UV: I=214 nm) using both MeCN t_{R} 12.00 (5-60% MeCN in H₂O, 0.1% FA, 25 min gradient) and MeOH t_{R} 19.49 (0-60% MeOH in H₂O, 0.1% FA, 25 min gradient) and revealed >99% purity. HRMS Calcd. m/z for C₃₆H₅₇O₁₁N₈ [M+H]⁺ 777.4141. found 777.4138.

### Example 44

### D-Arg-(S)-Bgl-D-Thr-D-Val-D-Glu-D-Leu-D-Ala-NH₂ 82

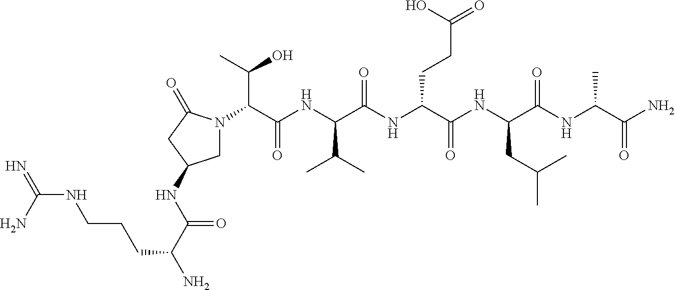

D-Arg-(S)-Bgl-D-Thr-D-Val-D-Glu-D-Leu-D-Ala-NH₂ 82 was prepared on a 0.105 mmol scale, in a syringe tube following the optimized protocol as described above, using microwave assisted annulation over 600 min, to give the desired lactam peptide TFA salt 82 (33 mg) in 82% crude purity as determined by analytical RP-HPLC (column a, UV: I=214 nm, 0-20% MeCN in H₂O, 0.1% FA, 30 min gradient). Purification was carried out by preparative RP-HPLC (column A, 0-20% MeCN in H₂O, 0.1% FA, 30 min gradient) to give the desired FA salt product 82 (13 mg, 16%) as a white fluffy solid. The purified product was analyzed by analytical RP-HPLC (column a, UV: I=214 nm) using both MeCN t_{R} 7.14 (5-60% MeCN in H₂O, 0.1% FA, 25 min gradient) and MeOH t_{R} 8.62 (0-80% MeOH in H₂O, 0.1% FA, 25 min gradient) and revealed >99% purity. HRMS Calcd. m/z for C₃₃H₆₀O₁₀N₁₁ [M+H]⁺ 770.4519. found 770.4518.

### Example 45

### D-Arg-D-Tyr-(S)-Bgl-D-Val-D-Glu-D-Leu-D-Ala-NH₂ 83

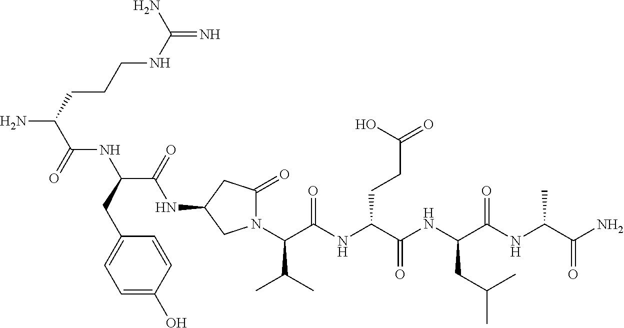

D-Arg-D-Tyr-(S)-Bgl-D-Val-D-Glu-D-Leu-D-Ala-NH₂ 83 was prepared on a 0.105 mmol scale, in a syringe tube following the optimized protocol as described above, using microwave assisted annulation over 480 min, to give the desired lactam peptide TFA salt 83 (52 mg) in 60% crude purity as determined by analytical RP-HPLC (column a, UV: I=214 nm, 0-80% MeCN in H₂O, 0.1% FA, 30 min gradient). Purification was carried out by preparative RP-HPLC (column A, 5-15% MeCN in H₂O, 0.1% FA, 50 min gradient) to give the desired FA salt product 83 (15 mg, 17%) as a white fluffy solid. The purified product was analyzed by analytical RP-HPLC (column a, UV: I=214 nm) using both MeCN t_{R} 9.43 (5-60% MeCN in H₂O, 0.1% FA, 25 min gradient) and MeOH t_{R} 14.71 (0-60% MeOH in H₂O, 0.1% FA, 25 min gradient) and revealed >99% purity. HRMS Calcd. m/z for C₃₈H₆₂O₁₀N₁₁ [M+H]⁺ 832.4676. found 832.4674.

### Example 46

### D-Arg-D-Tyr-D-Thr-(S)-Bgl-D-Glu-D-Leu-D-Ala-NH₂ 84

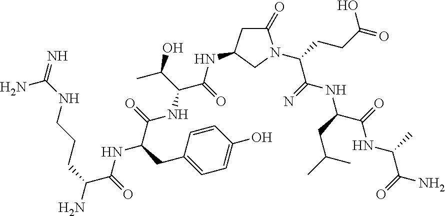

D-Arg-D-Tyr-D-Thr-(S)-Bgl-D-Glu-D-Leu-D-Ala-NH₂ 84 was prepared on a 0.105 mmol scale, in a syringe tube following the optimized protocol as described above, using microwave assisted annulation over 420 min, to give the desired lactam peptide TFA salt 84 (28 mg) in 87% crude purity as determined by analytical RP-HPLC (column a, UV: I=214 nm, 0-80% MeCN in H₂O, 0.1% FA, 30 min gradient). Purification was carried out by preparative RP-HPLC (column A, 0-20% MeCN in H₂O, 0.1% FA, 20 min gradient) to give the desired FA salt product 84 (12 mg, 14%) as a white fluffy solid. The purified product was analyzed by analytical RP-HPLC (column a, UV: I=214 nm) using both MeCN t_{R} 7.56 (5-60% MeCN in H₂O, 0.1% FA, 25 min gradient) and MeOH t_{R} 8.91 (0-80% MeOH in H₂O, 0.1% FA, 25 min gradient) and revealed >99% purity. HRMS Calcd. m/z for C₃₇H₆₀O₁₁N₁₁ [M+H]⁺ 834.4468. found 834.4469.

### Example 47

### D-Arg-D-Tyr-D-Thr-D-Val-(S)-Bgl-D-Leu-D-Ala-NH₂ 85

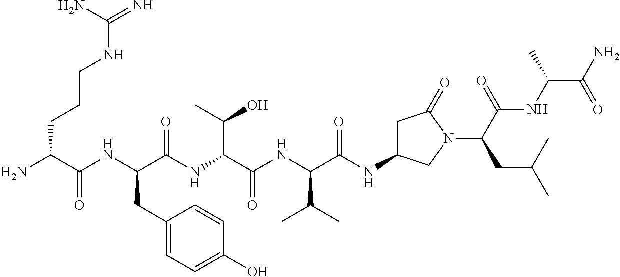

D-Arg-D-Tyr-D-Thr-D-Val-(S)-Bgl-D-Leu-D-Ala-NH₂ 85 was prepared on a 0.105 mmol scale, in a syringe tube following the optimized protocol as described above, using microwave assisted annulation over 240 min, to give the desired lactam peptide TFA salt 85 (40 mg) in 90% crude purity as determined by analytical RP-HPLC (column a, UV: I=214 nm, 0-80% MeCN in H₂O, 0.1% FA, 30 min gradient). Purification was carried out by preparative RP-HPLC (column A, 0-20% MeCN in H₂O, 0.1% FA, 30 min gradient) to give the desired FA salt product 85 (11 mg, 13%) as a white fluffy solid. The purified product was analyzed by analytical RP-HPLC (column a, UV: I=214 nm) using both MeCN t_{R} 9.48 (5-60% MeCN in H₂O, 0.1% FA, 25 min gradient) and MeOH t_{R} 10.47 (0-80% MeOH in H₂O, 0.1% FA, 25 min gradient) and revealed >99% purity. HRMS Calcd. m/z for C₃₇H₆₁O₉N₁₁ [M+H]⁺ 804.4726. found 804.4724.

### Example 48

### D-Arg-D-Tyr-D-Thr-D-Val-D-Glu-(S)-Bgl-D-Ala-NH₂ 86

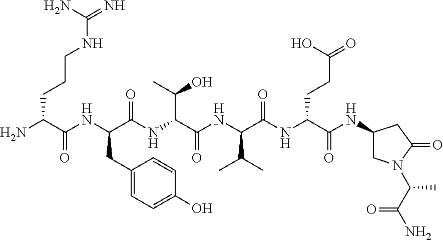

D-Arg-D-Tyr-D-Thr-D-Val-D-Glu-(S)-Bgl-D-Ala-NH₂ 86 was prepared on a 0.105 mmol scale, in a syringe tube following the optimized protocol as described above, using microwave assisted annulation over 240 min, to give the desired lactam peptide TFA salt 86 (29 mg) in 96% crude purity as determined by analytical RP-HPLC (column a, UV: I=214 nm, 0-20% MeCN in H₂O, 0.1% FA, 30 min gradient). Purification was carried out by preparative RP-HPLC (column A, 0-20% MeCN in H₂O, 0.1% FA, 30 min gradient) to give the desired FA salt product 86 (10 mg, 11%) as a white fluffy solid. The purified product was analyzed by analytical RP-HPLC (column a, UV: I=214 nm) using both MeCN t_{R} 7.6 (5-60% MeCN in H₂O, 0.1% FA, 25 min gradient) and MeOH t_{R} 8.9 (0-80% MeOH in H₂O, 0.1% FA, 25 min gradient) and revealed >99% purity. HRMS Calcd. m/z for C₃₆H₅₈O₁₁N₁₁ [M+H]⁺ 820.4131. found 820.4136.

### (R)-Bgl Insertion 101.10 Compounds

### Example 49

### D-Arg-(S)-Bgl-D-Tyr-D-Thr-D-Val-D-Glu-D-Leu-D-Ala-NH₂ 87

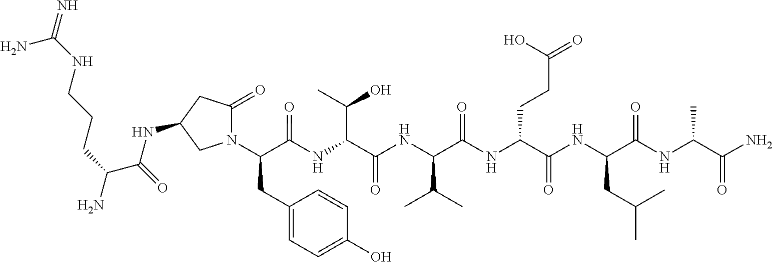

D-Arg-(S)-Bgl-D-Tyr-D-Thr-D-Val-D-Glu-D-Leu-D-Ala-NH₂ 87 was prepared on a 0.048 mmol scale, in a syringe tube following the optimized protocol as described above, using microwave assisted annulation over 600 min, to give the desired lactam peptide TFA salt 87 (27 mg) in 56% crude purity as determined by analytical RP-HPLC (column a, UV: I=214 nm, 0-40% MeCN in H₂O, 0.1% FA, 30 min gradient). Purification was carried out by preparative RP-HPLC (column A, 2-20% MeCN in H₂O, 0.1% FA, 25 min gradient) to give the desired FA salt product 87 (9 mg, 18%) as a white fluffy solid. The purified product was analyzed by analytical RP-HPLC (column a, UV: I=214 nm) using both MeCN t_{R} 10.23 (5-60% MeCN in H₂O, 0.1% FA, 25 min gradient) and MeOH t_{R} 15.99 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5152.

### Example 50

### D-Arg-D-Tyr-D-(S)-Bgl-Thr-D-Val-D-Glu-D-Leu-D-Ala-NH₂ 88

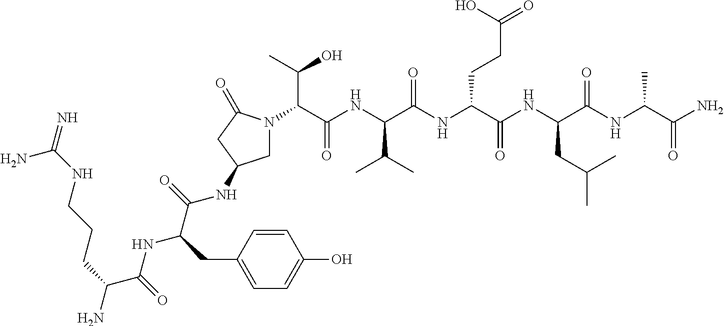

D-Arg-D-Tyr-D-(S)-Bgl-Thr-D-Val-D-Glu-D-Leu-D-Ala-NH₂ 88 was prepared on a 0.052 mmol scale, in a syringe tube following the optimized protocol as described above, using microwave assisted annulation over 600 min, to give the desired lactam peptide TFA salt 88 (6 mg) in 55% crude purity as determined by analytical RP-HPLC (column b, UV: I=214 nm, 0-60% MeCN in H₂O, 0.1% FA, 25 min gradient). Purification was carried out by preparative RP-HPLC (column B, 2-15% MeCN in H₂O, 0.1% FA, 30 min gradient) to give the desired FA salt product 88 (3 mg, 6%) as a white fluffy solid. The purified product was analyzed by analytical RP-HPLC (column b, UV: I=214 nm) using both MeCN t_{R} 9.22 (0-60% MeCN in H₂O, 0.1% FA, 25 min gradient) and MeOH t_{R} 14.01 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5137.

### Example 51

### D-Arg-D-Tyr-D-Thr-(S)-Bgl-D-Val-D-Glu-D-Leu-D-Ala-NH₂ 89

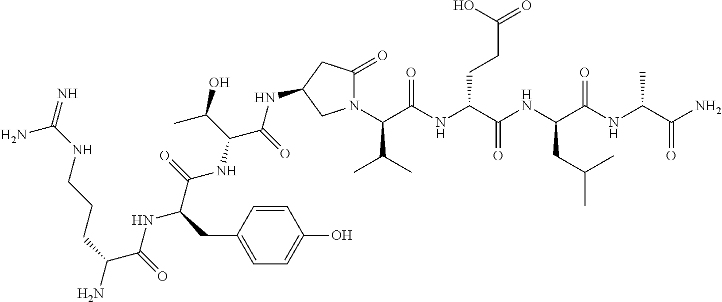

D-Arg-D-Tyr-D-Thr-(S)-Bgl-D-Val-D-Glu-D-Leu-D-Ala-NH₂ 89 was prepared on a 0.055 mmol scale, in a syringe tube following the optimized protocol as described above, using microwave assisted annulation over 480 min, to give the desired lactam peptide TFA salt 89 (16 mg) in 71% crude purity as determined by analytical RP-HPLC (column a, UV: I=214 nm, 0-60% MeCN in H₂O, 0.1% FA, 25 min gradient). Purification was carried out by preparative RP-HPLC (column A, 2-20% MeCN in H₂O, 0.1% FA, 20 min gradient) to give the desired FA salt product 89 (6 mg, 10%) as a white fluffy solid. The purified product was analyzed by analytical RP-HPLC (column a, UV: I=214 nm) using both MeCN t_{R} 10.38 (5-60% MeCN in H₂O, 0.1% FA, 25 min gradient) and MeOH t_{R} 16.25 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5152.

### Example 52

### D-Arg-D-Tyr-D-Thr-D-Val-(S)-Bgl-D-Glu-D-Leu-D-Ala-NH₂ 90

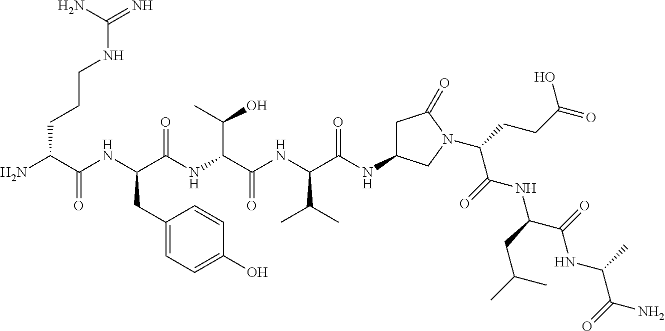

D-Arg-D-Tyr-D-Thr-D-Val-(S)-Bgl-D-Glu-D-Leu-D-Ala-NH₂ 90 was prepared on a 0.058 mmol scale, in a syringe tube following the optimized protocol as described above, using microwave assisted annulation over 420 min, to give the desired lactam peptide TFA salt 90 (7 mg) in 68% crude purity as determined by analytical RP-HPLC (column b, UV: I=214 nm, 5-30% MeCN in H₂O, 0.1% FA, 30 min gradient). Purification was carried out by preparative RP-HPLC (column B, 5-15% MeCN in H₂O, 0.1% FA, 30 min gradient) to give the desired FA salt product 90 (3 mg, 6%) as a white fluffy solid. The purified product was analyzed by analytical RP-HPLC (column b, UV: I=214 nm) using both MeCN t_{R} 9.90 (5-60% MeCN in H₂O, 0.1% FA, 25 min gradient) and MeOH t_{R} 15.20 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5147.

### Example 53

### D-Arg-D-Tyr-D-Thr-D-Val-D-Glu-(S)-Bgl-D-Leu-D-Ala-NH₂ 91

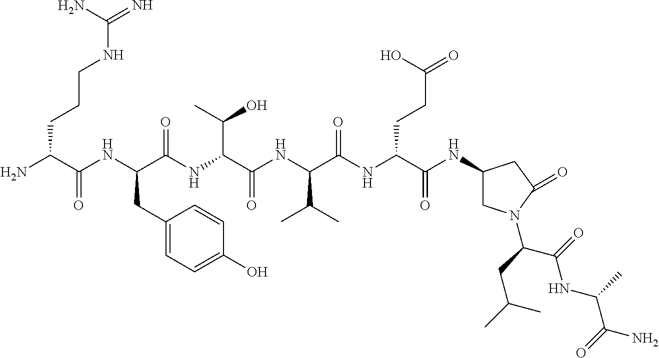

D-Arg-D-Tyr-D-Thr-D-Val-D-Glu-(S)-Bgl-D-Leu-D-Ala-NH₂ 91 was prepared on a 0.065 mmol scale, in a syringe tube following the optimized protocol as described above, using microwave assisted annulation over 240 min, to give the desired lactam peptide TFA salt 91 (20 mg) in 37% crude purity as determined by analytical RP-HPLC (column b, UV: I=214 nm, 2-30% MeCN in H₂O, 0.1% FA, 25 min gradient). Purification was carried out by preparative RP-HPLC (column B, 5-15% MeCN in H₂O, 0.1% FA, 25 min gradient) to give the desired FA salt product 91 (2 mg, 3%) as a white fluffy solid. The purified product was analyzed by analytical RP-HPLC (column b, UV: I=214 nm) using both MeCN t_{R} 9.96 (5-60% MeCN in H₂O, 0.1% FA, 25 min gradient) and MeOH t_{R} 15.43 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5154.

### Example 54

### D-Arg-D-Tyr-D-Thr-D-Val-D-Glu-D-Leu-(S)-Bgl-D-Ala-NH₂ 92

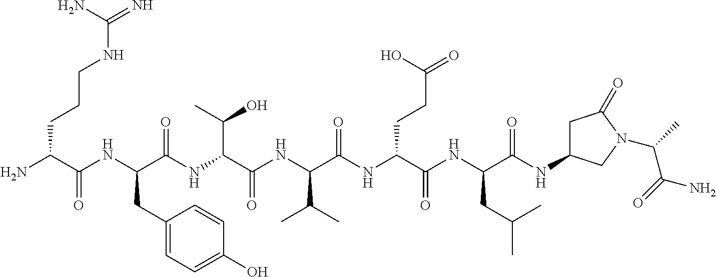

D-Arg-D-Tyr-D-Thr-D-Val-D-Glu-D-Leu-(S)-Bgl-D-Ala-NH₂ 92 was prepared on a 0.070 mmol scale, in a syringe tube following the optimized protocol as described above, using microwave assisted annulation over 240 min, to give the desired lactam peptide TFA salt 92 (27 mg) in 97% crude purity as determined by analytical RP-HPLC (column a, UV: I=214 nm, 5-30% MeCN in H₂O, 0.1% FA, 30 min gradient). Purification was carried out by preparative RP-HPLC (column A, 2-30% MeCN in H₂O, 0.1% FA, 30 min gradient) to give the desired FA salt product 92 (13 mg, 18%) as a white fluffy solid. The purified product was analyzed by analytical RP-HPLC (column a, UV: I=214 nm) using both MeCN t_{R} 10.04 (5-60% MeCN in H₂O, 0.1% FA, 25 min gradient) and MeOH t_{R} 15.69 (0-60% MeOH in H₂O, 0.1% FA, 25 min gradient) and revealed >99% purity. HRMS Calcd. m/z for C₄₂H₆₉O₁₂N₁₂ [M+H]⁺ 933.5152. found 933.5150.

Discussion for Examples 32-54

After cleavage of compounds 54, 55, 56 and 57, where a (R)-Agl lactam respectively replaced D-Arg¹, D-Tyr², D-Thr³ and D-Val⁴, measurable amounts of products resulting from incomplete lactamization were observed in the corresponding crude mixtures. In place of an Agl residue, compounds 71, 72, 73 and 74 possessed a (R)-benzyl-2-aminobut-4-yloate moiety [(R)-Bab]. A general process for the obtention of (R)-Bab containing peptides is depicted in Scheme 8.

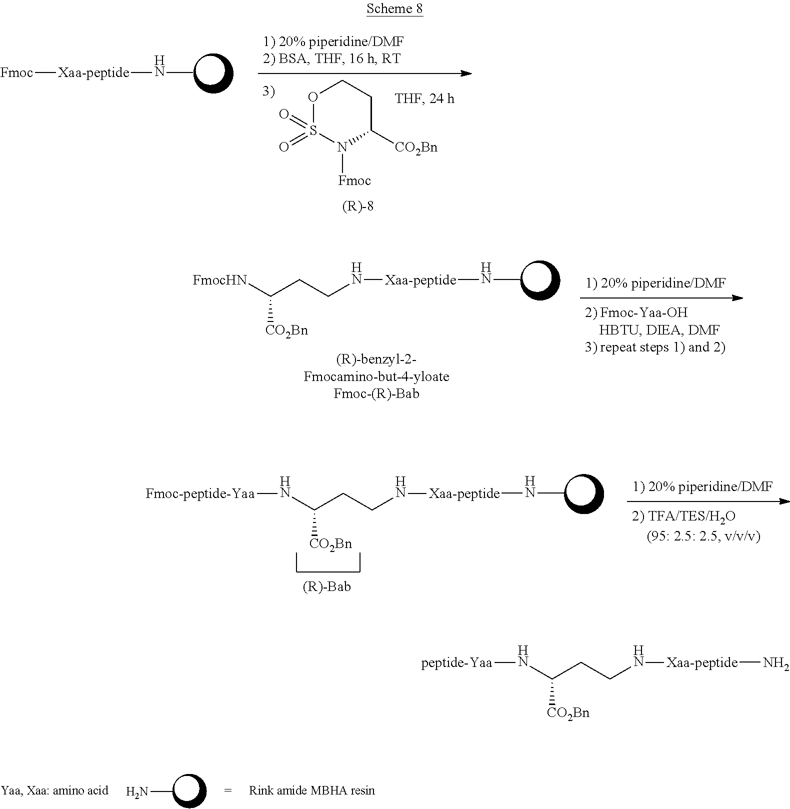

These side-products from incomplete lactam formation could easily be separated from the corresponding lactam compounds and were isolated during purification by RP-HPLC (Table 6).

In order to further broaden the scope of the process of the present application and in search of new active IL1-RI modulating compounds, a (S)- and (R)-Bgl scan of 101.10 was performed using the optimized protocol developed in studies of GHRP-6 with (R)-Bgl (see Scheme 9, Table 7).

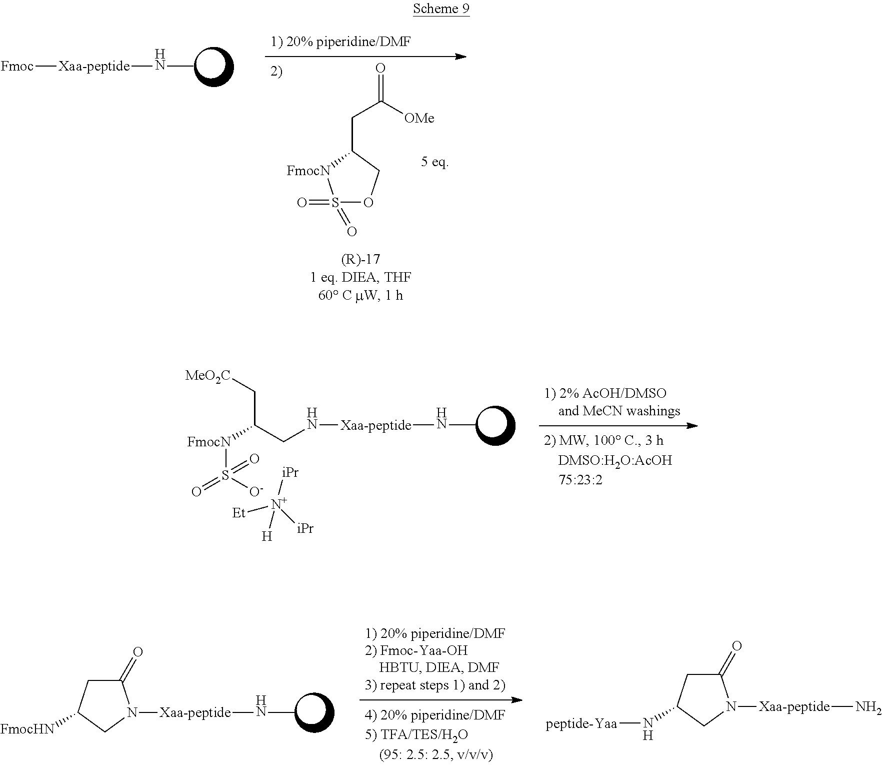

Microwave assisted acylation required longer reaction times (>4 h) in the synthesis of analogs 76, 78, 81-84, presumably due to the difficulties of cyclization on sterically bulky β-alkyl branched amino acids and residues carrying tert-butyl ether and ester protected side chains. Moreover the configuration of the diastereomers influenced the rate of lactam formation of Bgl on the D-Tyr(OtBu) residue requiring 10 h of microwave heating in the (S) case (81) and a standard time of 4 h in the (R) case (75), to achieve 90% completion. This was also the case of compound 76 where the (R)-Bgl lactamization on D-Thr(O-tBu) took 6 h whereas for compound 81, (S)-Bgl lactamization required 10 h. On the other hand, lactam formation on D-Glu(OtBu) required 7 h of microwave heating in both cases (R, 78; S, 83). In the alkylation step, no diastereoisomeric effect was apparent and alkylation proceeded with 20% conversion on D-Ala up to 90% conversion for alkylation on D-Tyr(OtBu), in both (R) and (S) cases. Proximity to solid support seemed to be the controlling parameter for success of the reaction, the best yields being obtained when the alkylated residue is furthest from the polymer matrix. In every case, bis-alkylation compounds were not detected or their amounts were <10% of UV total signal at 214 nm, as measured by RP-HPLC analysis of a cleaved sample. Contrary to GHRP-6 featuring a Lys side chain protected as a Boc group, permanent protective groups were stable to microwave heating condition, thus a Boc re-protection step was not necessary. Nevertheless an acetyl capping was still performed, instead of Boc capping, after lactamisation, leading to better crude purities than in the case of lactam peptides produced in study of GHRP-6, acetylated compounds, resulting from unsuccessful alkylation, being washed out during ether precipitation after cleavage.

Introduction of the Bab residue in 101.10 led to compounds with improved efficacy in inhibiting IL-1 induced TF-1 cell proliferation. To investigate the features brought by Bab that are beneficial to 101.10 activity, it was decided to use Bgl as a spacer, intercalating it between each residue in an “insertion scan”. A (S)-Bgl insertion scan was performed on 101.10 using the optimized protocol as described (Table 8).

### Example 55

### Membrane Preparation for GHS-R1a Receptor

Transfection LLC-PK1 cells were seeded at 1.5×10⁶ cells/10 cm Petri-dishes and grown for 24 h in DMEM high-glucose (4.5 g/L) with 10% foetal bovine serum supplemented with penicillin (10,000 Units/ml) and streptomycin (10,000 ug/ml), and cultured at 37° C., with 5% CO₂. The medium was then replaced for another 4-5 hours before CaPO₄ calcium phosphate transfection. The DNA solution consists of 40 μg of DNA in a volume of 500 μl in which was added 500 μl of 2 mM Tris-HCl pH 8.0, 0.2 mM EDTA pH 8.0 containing 500 mM CaCl₂, to a final volume of 1 ml. Then 1 ml of 50 mM Hepes, 280 mM NaCl, 1.5 mM Na₂HPO₄ pH to 7.1 (HBSS) was added by alterning 1 drop/2 air bubbles. The transfection mix reaction was incubated at RT for 30 min. After the incubation period, 1 ml of the mix was added to each plate and distributed evenly for incubation. The media was replaced with standard DMEM-high glucose media for another 24 h and cells were collected for membrane preparation.

Membrane preparation The experiment was carried out 4° C. unless specified. Cells were washed 2 times with PBS and with the homogenization buffer (HB) consisting of 50 mM Tris, 5 mM MgCl₂, 2.5 mM EDTA, 30 ug/ml bacitracin at pH 7.3 and were scraped in Eppendorf tubes. Cells were lysed with 2 cycles of freeze/thawing using liquid nitrogen and were then centrifuged at 4° C. for 20 min at 10,000 g to collect the membranes. The membranes were re-suspended in a small volume of HB and aliquoted for storage at −80° C.

### Example 56

### GHS-R1a Receptor Binding Assay

The competitive binding assay consists of 200 μl HB, 1041 ¹²⁵I-Ghrelin (40,000 cpm), 100 μl competitive ligand (from 10⁻¹² to 10⁻⁵M) and 100 μl of GHS-R1a transiently transfected in LLC-PK1 cells as source of binding sites (10 μg protein/tube). The non-specific binding was determined by excess of competitive ligand at 10⁻⁵M. The reaction was carried out at RT for 1 h. After the incubation period, the separation of bound from free fraction was performed by filtration over a GF/C filter pre-soaked with 0.5% polyethyleneimine and the filters were washed with 4 ml of HB consisting of 50 mM Tris, 10 mM MgCl₂, 2.5 mM EDTA and 3 ml of wash buffer consisting of 50 mM Tris-10 mM MgCl₂, 20 mM EDTA, 0.015% Triton X100 (pH 7.3) and were then collected for radioactivity counting using gamma counter (LKB Wallac 1277, Turku Finland).

### Example 57

### Membrane Preparation for CD36

Animal use was in accordance with the Institutional Animal Ethics Committee and the Canadian Council on Animal Care guidelines for the use of experimental animals. Sprague Dawley (275-350 g) rats were anaesthetized with sodium pentobarbital and their hearts were promptly removed in ice-cold saline and the cardiac membranes were prepared according to Harigaya and Schwartz.^{lxvi}

### Example 58

### Competitive Covalent CD36 Binding Assay Using Photoactivatable [¹²⁵I]-Tyr-Bpa-Ala-Hexarelin as Radioligand

The radioiodination procedure of the photoactivatable ligand and the receptor binding assays were performed as previously described by Ong et al.^{lxvii} Briefly, the rat cardiac membranes (200 μg) as source of CD36 were incubated in the darkness, in 525 μl of 50 mM Tris-HCl pH 7.4 containing 2 mM EGTA (Buffer A) in the presence of a fixed concentration of [¹²⁵I]-Tyr-Bpa-Ala-Hexarelin (750 000 cpm) in Buffer B (50 mM Tris-HCl pH 7.4 containing 2 mM EGTA and 0.05% Bacitracin) and with increasing concentrations of competitive ligands (ranging from 0.1 to 50 μM). Nonspecific binding was defined as binding not displaced by 50 μM peptide. After an incubation period of 60 min at 22° C., membranes were submitted to UV irradiation at 365 nm for 15 min at 4° C. After centrifugation at 12 000 g for 15 min, the pellets were resuspended in 100 μl of sample buffer consisting of 62 mM Tris-HCl, pH 6.8, 2% SDS, 10% glycerol, 15% 2-mercapto-ethanol, and 0.05% bromophenol blue, and boiled for 5 min prior to being subjected to electrophoresis on 7.5% SDS-PAGE. The SDS/PAGE gels were fixed, colored in Coomassie Brilliant Blue R-250, dried, exposed to a storage phosphor intensifying screen (Amersham Biosciences), and analysed by using a Typhoon PhosphorImager (Amersham Biosciences) and ImageQuant 5.0 software to establish competition curves. Protein bands corresponding to the specifically labeled protein of 87 kDa were quantified by densitometry analysis.

### Example 59

### [³H]thymidine Incorporation for TF-1 Cell Proliferation Measurements

Human TF-1 cells (5×10⁴ cells/well) were cultured in complete RPMI medium (GIBCO RPMI Medium 1640, Invitrogen) supplemented with GM-CSF (Granulocyte Macrophage Colony Stimulating Factor, 2 ng/ml; BD Biosource). Cells were deprived of growth factors for 18 h before preincubation with lactam peptide (1 μM) followed by treatment with IL-1β (10 or 25 ng/mL). After 24 h incubation at 37° C., [³H]thymidine (1 μCi/mL; Amersham) was added and the cells were incubated for another 24 h. Cells were harvested, washed two times with PBS (10 mM Na₂HPO₄, 2 mM KH₂PO₄, 137 mM NaCl, 2.7 mM KCl; pH 7.4) and lysed with a 0.1 N NaOH/0.1% Triton X-100 solution. Scintillation cocktail (Fisher Scientific, 8 mL/sample) was added to the lysate, and after 3 h, radioactivity was measured (Beckman Multi-Purpose Scintillation Coulter Counter LS6500). Results were analyzed by one- or two-way ANOVA factoring for concentration or treatments. Postanova comparisons among means were performed using the Tukey-Framer method. Statistical significance was set at p<0.05. Data are presented as mean±SEM.

Discussion for Examples 55-59

Binding Affinity IC₅₀ Values of GHRP-6 Lactam Analogs 44-53 and 60-70 on GHS-R1a and CD36 Receptors

The affinity of Agl- and Bgl-containing GHRP-6 analogs 44-53 was assessed in competition studies with an iodinated [¹²⁵I] analog of the native GHS-R1a ligand ghrelin on cells transiently transfected with the receptor. Similarly, displacement of labeled [¹²⁵I]-Tyr-Bpa-Ala-Hexarelin by Agl- and Bgl-peptides 44-53 was examined on rat cardiac membranes rich in CD-36 receptors (Table 4).^{lxvi,lxvii}

Most lactam analogs lost affinity for both the GHS-R1a and CD36 receptors; however, lactam 46, in which Ala³ was replaced by (S)-Agl, retained similar affinity for the CD36 receptor, yet lost binding ability to the GHS-R1a receptor by a factor of 10³ relative to GHRP-6. In the cases of 48 and 53, in which D-Phe⁵ was respectively replaced by (S)-Agl and (S)-Bgl, and 45 where D-Trp² was substituted by (S)-Agl, the influence of the lactam moiety was similarly more significant on the GHS-R1a receptor, for which affinity decreased by a factor of 10³-10⁴, rather than the CD36 receptor for which affinity dropped by a factor of 15. Considering that both Agl and Bgl may induce turn conformations, the affinity of lactam analogs of GHRP-6 supports the hypothesis for a β-turn conformation about residues 2-4, which may be responsible for binding and differentiating the CD36 receptor from the GHS-1Ra receptor. Finally, replacement of the His¹ residue with (S)-Agl and (S)-Bgl (compounds 44 and 49) did not cause complete loss of receptor affinity, which was consistent with previous analogs featuring substitutions at this position.^{lxviii}

The affinity of (R)-amino lactam peptides 60-70 towards receptors GHS-R1a and CD36 were also assessed. The results are presented in Table 5. Similarly to their (S)-lactam counterparts, most (R)-lactam analogs lost binding affinity for both the GHS-R1a and CD36 and in most instances, affinity loss is greater for (R)-lactam peptides. This is especially the case concerning the GHS-R1a receptor, which seems more sensitive to configurational changes within its ligands than CD36. Whereas binding affinity towards GHS-R1a of compound 44, where His¹ was substituted by (S)-Agl, only decreased by a 10 factor compared to GHRP6, this one decreased by 2.10⁻³ with the corresponding compound 60. The later result suggests a limitation to the extent to which position 6 can be modified without affecting binding with GHS-R1a. Compounds 64 and 69 in which D-Phe⁵ and Trp⁴ were respectively replaced by a (R)-Agl and a (R)-Bgl, kept some binding affinity with the CD36 receptor, although reduced by a 10 factor compared with hexarelin, making them selective binding partners of this receptor.

Inhibition of Thymocytes TF-1 Proliferation by 101.10 Lactam Analogs 54-59

The efficacy of Agl peptides 54-59 was ascertained by measuring their influence on IL-1 induced human thymocyte TF-1 proliferation as assessed by incorporation of [³H]thymidine as previously described.

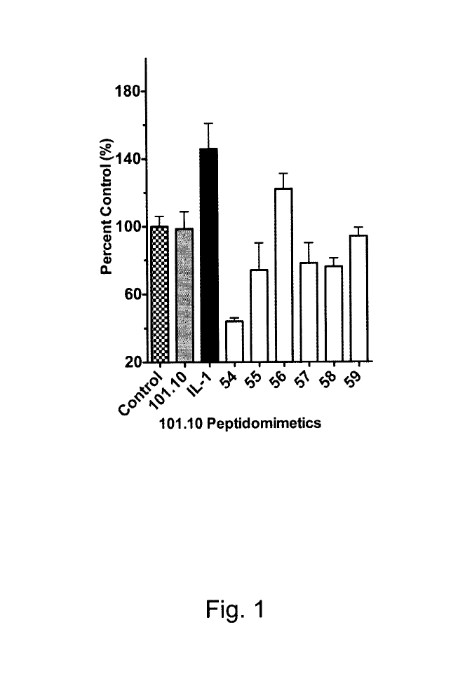

Among the analogs tested, five maintained some inhibitory effect on TF-1 proliferation (FIG. 1). Peptide 56 failed to block proliferation of TF-1 cells treated with IL-1. Relative to 101.10, lactams 59, 58, 57, and 55, all exhibited similar efficacy, suggesting that the balance between side chain removal and conformational constraint at positions 2 and 4-6 does not perturb activity. Replacement of the N-terminal D-Arg residue by (R)-Agl in compound 54 led to 2.2 fold increase in efficacy compared to 101.10, suggesting that the Arg¹ side chain may not be necessary for activity.

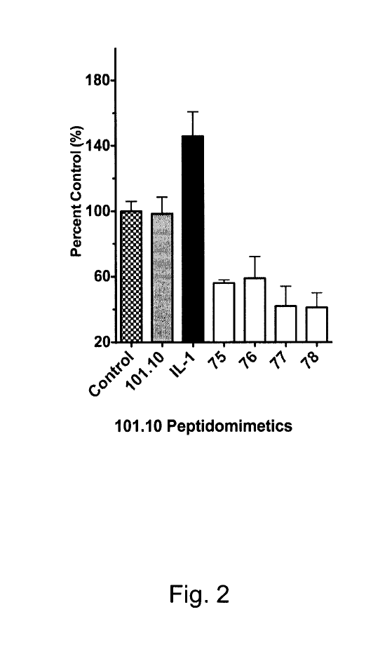

The efficacy of Agl peptides 75-78 as allosteric negative modulators of the IL-1 receptor I was also ascertained by measuring their influence on IL-1 induced human thymocyte TF-1 proliferation as assessed by incorporation of [³H]thymidine (FIG. 2).^{xlixa}

Results show that all (R)-Bab analogs tested exhibited an improvement in efficacy compared to 101.10, from a 2.4 fold (compound 78) to a 1.7 fold (compound 76) increase. Inhibition of TF-1 proliferation obtained with compound 75 confirms that the Arg¹ side chain does not seem to be necessary for activity. Reasons for the increase of efficacy observed with compounds 76-78 remains unclear and requires further investigation regarding the influence of features brought by a Bab residue, which, while not wishing to be limited by theory, may include the character of the aromatic ester and the increased conformational flexibility from insertion of an amino alkyl chain into the peptide backbone.

### Example 60

### Preparation of Further Peptides Comprising Agl and Bgl

The following additional peptides were prepared using the process of the present application:

### (a) D-Agl-D-Tyr-Iaa-D-Glu-D-Leu-NH₂ Iaa=(3R,6R,9R)-3-amino-indolizidin-2-one-9-carboxylic acid

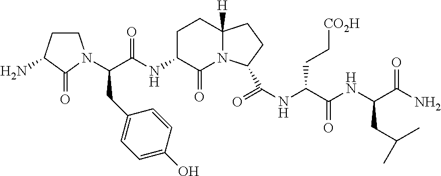

D-Agl-D-Tyr-Iaa-D-Glu-D-Leu-NH₂ was prepared as the desired peptide TFA salt (34.0 mg, 44% crude purity as analyzed by analytical RP-HPLC (UV 214), 0-40 MeCN, 8 min gradient). Purification was then carried out by preparatory RP-HPLC (2-40 MeCN, 25 min gradient) to give the desired formic acid salt WC144 (4.4 mg, 6%) as a white foam. The purified product was analyzed by analytical RP-HPLC (UV 214) using both MeCN t_{R} 16.47 (2-40 MeCN, 25 min gradient) and MeOH t_{R} 19.14 (0-60 MeOH, 25 min gradient) and revealed >96% purity. HRMS Calcd. for C₃₃H₄₈O₉N₇ [M+H]⁺ 686.3508. found 686.3512.

### (b) Agl-D-Tyr-Iaa-D-Glu-D-Leu-NH₂

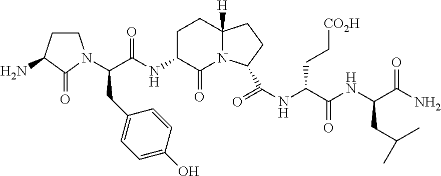

Agl-D-Tyr-Iaa-D-Glu-D-Leu-NH₂ was prepared as the desired peptide TFA salt (30.0 mg, 39% crude purity as analyzed by analytical RP-HPLC (UV 214), 0-40 MeCN, 8 min gradient). Purification was then carried out by preparatory RP-HPLC (2-40 MeCN, 25 min gradient) to give the desired formic acid salt WC145 (5.0 mg, 6%) as a white foam. The purified product was analyzed by analytical RP-HPLC (UV 214) using both MeCN t_{R} 16.18 (2-40 MeCN, 25 min gradient) and MeOH t_{R} 18.73 (0-60 MeOH, 25 min gradient) and revealed >99% purity. HRMS Calcd. for C₃₃H₄₈O₉N₇ [M+H]⁺ 686.3508. found 686.3514.

### (c) D-Bgl-D-Tyr-Iaa-D-Glu-D-Leu-NH₂

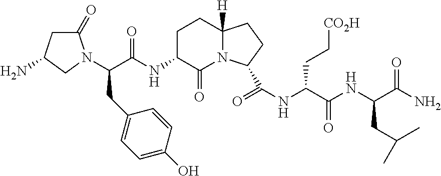

D-Bgl-D-Tyr-Iaa-D-Glu-D-Leu-NH₂ was prepared as the desired peptide TFA salt (37.0 mg, 48% crude purity as analyzed by analytical RP-HPLC (UV 214), 0-40 MeCN, 8 min gradient). Purification was then carried out by preparatory RP-HPLC (2-40 MeCN, 25 min gradient) to give the desired formic acid salt WC146 (10.5 mg, 13%) as a white foam. The purified product was analyzed by analytical RP-HPLC (UV 214) using both MeCN t_{R} 15.85 (2-40 MeCN, 25 min gradient) and MeOH t_{R} 18.80 (0-60 MeOH, 25 min gradient) and revealed >98% purity. HRMS Calcd. for C₃₃H₄₈O₉N₇ [M+H]⁺ 686.3508. found 686.3514.

### (d) Bgl-D-Tyr-Iaa-D-Glu-D-Leu-NH₂

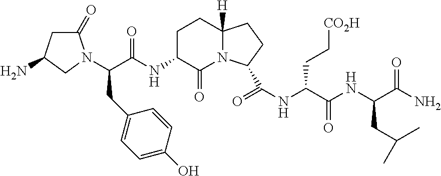

Bgl-D-Tyr-Iaa-D-Glu-D-Leu-NH₂ was prepared as the desired peptide TFA salt (37.0 mg, 48% crude purity as analyzed by analytical RP-HPLC (UV 214), 0-40 MeCN, 8 min gradient). Purification was then carried out by preparatory RP-HPLC (2-40 MeCN, 25 min gradient) to give the desired formic acid salt WC147 (9.8 mg, 13%) as a white foam. The purified product was analyzed by analytical RP-HPLC (UV 214) using both MeCN t_{R} 16.04 (2-40 MeCN, 25 min gradient) and MeOH t_{R} 18.83 (0-60 MeOH, 25 min gradient) and revealed >99% purity. HRMS Calcd. for C₃₃H₄₈O₉N₇ [M+H]⁺ 686.3508. found 686.3508.

### Example 61

### Synthesis of D-Arg-D-Tyr-(S)-Agl-D-Val-D-Glu-D-Leu-D-Ala-NH₂ (93): Lantern-Resin Comparison and Synthesis of Multiple Lactam Residues on SynPhase Lanterns

### (a) Rink Amide-MBHA Resin

Swelling and Fmoc-Deprotection

A 12 mL plastic filtration tube with polyethylene frit was charged with Rink Amide-MBHA resin (200 mg, 0.134 mmol, 0.67 mmol/g) and DCM (7 mL). The tube was sealed and shaken for 0.5 h. The resin was then filtered and taken up in freshly prepared 20% piperidine in DMF solution (7 mL), shaken for 30 min, filtered, retreated with 20% piperidine/DMF solution (7 mL) and shaken for 30 min. A positive Kaiser colour test indicated qualitatively the presence of free amine.^{lix}

Washing

Washing steps after coupling or deprotection steps were performed by successive agitations for 1 min and filtration from DMF (3×7 mL), MeOH (3×7 mL) and DCM (3×7 mL).

Amino Acid Couplings

A solution of N-(Fmoc)amino acid (3 equiv.), HBTU (3 equiv.) and DIEA (6 equiv.) in DMF (7 mL) was prepared in a small sample vial, stirred for 3 min and then added to the resin. The reaction mixture was shaken for 1 h with Fmoc-D-Ala, for 3 h with Fmoc-D-Val, Fmoc-D-Thr(tBu), Fmoc-D-Tyr(tBu) and Fmoc-D-Arg(Pbf) and for 4 h with Fmoc-D-Leu, Fmoc-D-Glu(tBu), at room temperature. The completeness of each coupling was verified by the Kaiser test on few resin beads.^{lix}

Silylation and Alkylation

After Fmoc protecting group removal, resin was dried in vacuo for at least 3 h. The anhydrous resin, in a 12 mL plastic filtration tube with polyethylene frit, was flushed with argon, swollen in THF (7 mL), treated with BSA (5 equiv.), shaken for 6 h, filtered under argon and treated with a solution of sulfamidate (5 equiv.) in THF (7 mL). After shaking for 18 h at room temperature, the resin was filtered and washed under argon with THF (3×7 mL).

Microwave Assisted Annulation

A 2 mL glass microwave vial was charged with resin and a freshly prepared 1% AcOH/DMSO solution (2 mL). The vial was sealed, heated in the microwave at 110° C. (pressure 1 bar) for 6 h. The resin was then washed from the microwave vessel into a 12 mL plastic filtration tube with polyethylene frit.

Cleavage Test

Monitoring of reaction progress was performed by LC-MS analyses of material cleaved from resin. Typically, a small resin sample (3-5 mg) was treated with a mixture of TFA/H₂O/TES (1 mL, 95/2.5/2.5, v/v/v) for 1 h and filtered. The filtrate was evaporated, dissolved in water and acetonitrile and examined by LC-MS.

Final Cleavage

The peptide was cleaved from the resin by shaking in TFA/H₂O/TES (7 mL, 95/2.5/2.5, v/v/v) for 3 h. The resin was filtered and washed with TFA. The combined filtrate and washings were concentrated in vacuo. The resulting residue was dissolved in a minimum volume of TFA (˜1 mL), transferred to a centrifuge tube and precipitated by the addition of ice-cold diethyl ether (40 mL). The peptide was separated by centrifugation and the diethyl ether was carefully decanted from the tube. The precipitated peptide was washed twice with cold diethyl ether. The resulting white solid was dissolved in water and freeze-dried to give a white powder that was purified by preparative RP-HPLC, using the specified conditions.

### (b) Polystyrene Rink Amide Lanterns

D-Sized polystyrene Rink amide lanterns with a 35-μmol loading were used for the synthesis of 93 and 94 and A-sized polystyrene Rink amide lanterns with a 75-μmol loading were used for the synthesis of 95-97. A 20-mL sample vials with a cover in which seven 0.3 mm diameter holes were drilled was used and solutions were removed simply by reversing the flask.

Swelling and Fmoc-Deprotection

Swelling and Fmoc-deprotection steps were respectively performed by immersing lanterns for 30 min in DCM and in DMF/Pip (80/20, v/v) solution. A positive Kaiser colour test on a sliver of lantern indicated qualitatively the presence of free amine.^{lix}

Washing

Washing steps after coupling and deprotection steps were performed by dipping the lanterns in DMF (3×3 min), MeOH (1×3 min) and DCM (3×3 min), successively.

Amino Acid Coupling

A DMF solution containing the Fmoc-protected amino acid (3 equiv.), HBTU (3 equiv.), and DIEA (6 equiv.) were freshly prepared in a 20-mL flask and lanterns were immersed in the coupling solution for 3 h with Fmoc-D-Ala, Fmoc-D-Leu, Fmoc-D-Val, Fmoc-D-Tyr(tBu), Fmoc-D-Pro and Fmoc-D-Arg(Pbf) and for 4 h with Fmoc-D-Glu(tBu) and Fmoc-D-Thr(tBu), at room temperature. The completeness of each coupling was verified by a Kaiser test on a sliver of lantern.^{lix}

Silylation and Alkylation

After Fmoc protecting group deprotection, lanterns were then dried in vacuo for at least 3 h. The anhydrous lanterns, in 2- or 5-mL glass microwave vials, were flushed with argon, suspended in THF, treated with a solution of sulfamidate (4 equiv.) and DIEA (0-1.1 equiv) in THF and heated in the microwave at 60-70° C. for 1-2 h. The lanterns were washed under argon with THF. Alternately, the alkylation with sulfamidate was preceded by a treatment with BSA (5 equiv) in THF for 1-6 h.

Microwave Assisted Annulation

A 2- or 5-mL glass microwave vial was charged with lanterns and a freshly prepared solution of DMSO/AcOH (99:1, v/v) or DMSO/H₂O/AcOH (75:23:2, v/v/v). The vial was sealed, heated in the microwave at 80° C. for 5-10 h. The lanterns were then washed as previously described.

Capping

After lactam annulation, a capping of the deletion sequence from incomplete sulfamidate alkylation was performed by immersing the lantern in a DMF solution of acetic anhydride (10 equiv.) and DIEA (10 equiv) for 1 h.

Cleavage Test

Monitoring of reaction progress was performed by LC-MS analyses of material cleaved from lantern. Typically, a slice of lantern was treated with a mixture of TFA/H₂O/TES (1 mL, 95/2.5/2.5, v/v/v) for 1 h and filtered. The filtrate was evaporated, dissolved in water and acetonitrile and examined by LC-MS.

Peptide Cleavage

The peptide was cleaved by immersing the lantern in TFA/H₂O/TES (3 mL, 95/2.5/2.5, v/v/v) for 3 h. The cleavage cocktail was removed directly from the tubes, peptides were precipitated with ice-cold diethyl ether, centrifuged, and decanted. Precipitation, centrifugation, and decantation operations were repeated twice. The resulting white solid was dissolved in water (10 mL) and freeze-dried to give a white powder that was analysed for purity and purified by preparative RP-HPLC, using the specified conditions.

Discussion for Example 61

A comparative study was first made between Rink amide MBHA resin and Rink-amide SynPhase lantern as supports in the synthesis of lactam peptide 93, in which the D-Thr³ residue was replaced by (S)-Agl (Scheme 10). Assembly of the C-terminal peptide fragment (vela) was done by standard SPPS protocols^{lxiv}. The amino terminal of the peptide was then alkylated with (4S)-(Fmoc)oxathiazinane ester (S)-8, which along with its enantiomer (R)-8, were synthesized in solution from L- and D-Met as described above. Peptide alkylation was preceded by N-silylation of the peptide amine with N,O-bis(trimethylsilyl)acetamide (BSA) to minimize bis-alkylation. Lactam annulation was performed by microwave irradiation, the Fmoc group was removed and the sequence was elongated to provide the supported, protected Agl peptide 93. Lactam peptide 93 was obtained by side-chain deprotection and cleavage from the resin and lantern pon treatment with TFA. The crude peptides were analyzed by RP-HPLC and comparable crude purity was obtained using resin and lantern (33% and 32%, respectively) indicating that Agl peptide synthesis was unaffected by the support. After cleavage from the resin, the main impurities in crude material 93 were the deletion sequence from incomplete sulfamidate alkylation (30%) and uncyclized product (17%). After cleavage from lantern, the main impurity was the acetylated (S)-Agl-vela peptide (39%) probably due to an undesired partial Agl Fmoc deprotection prior to treatment with the acetic anhydride. The residual amine capping was performed on lantern after lactam annulation to avoid complications from a deletion sequence due to incomplete sulfamidate alkylation.

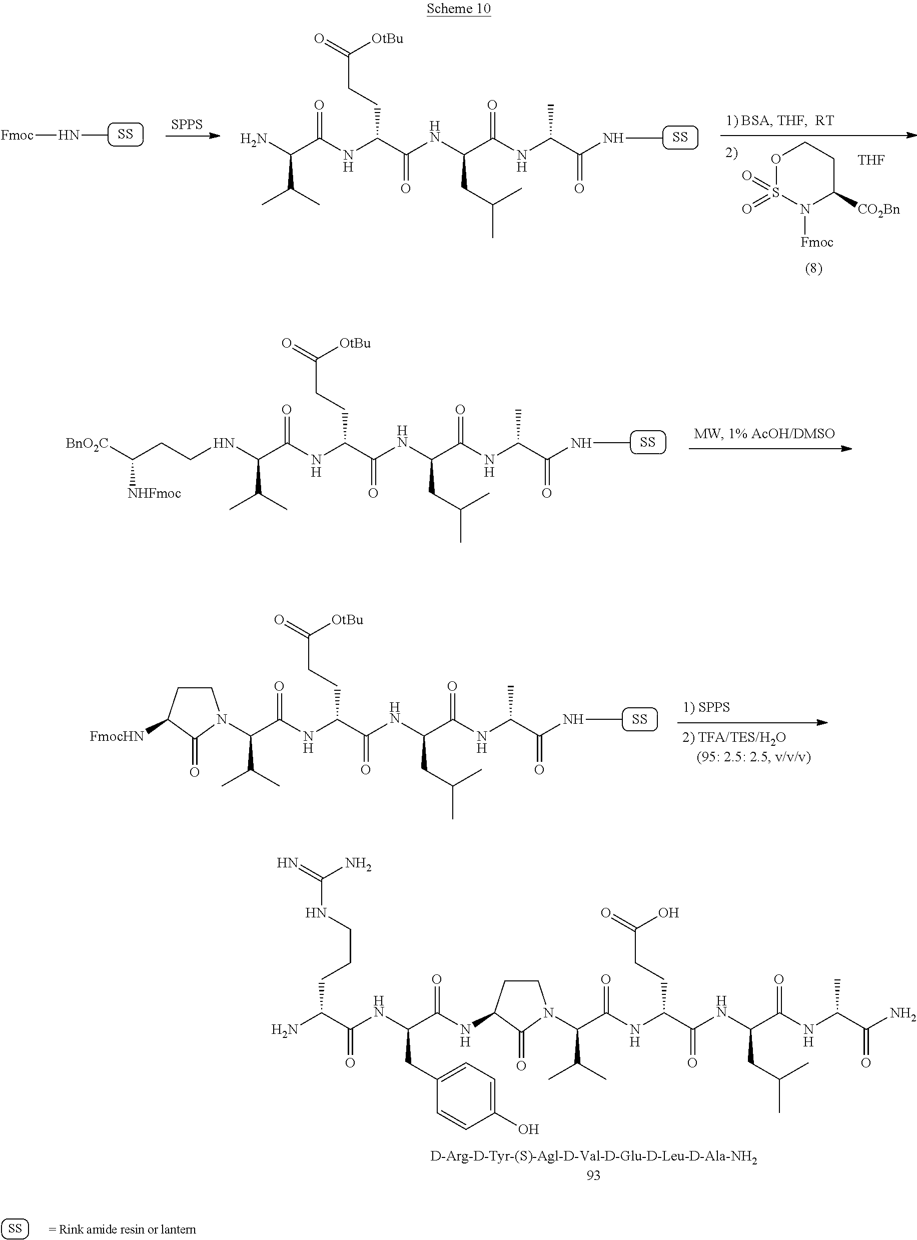

The rytvela analog 93 was synthesized in parallel on color-tagged lanterns with the analog 94, in which the D-Val⁴ residue was replaced by (S)-Agl. The C- and N-terminal peptide fragments were assembled by standard SPPS protocols. The alkylation with sulfamidate (S)-8 was performed after treatment of the peptide lanterns with BSA for 1 h, and comparable results were obtained by overnight alkylation at room temperature and heating at 60° C. with microwave-irradiation for 1 h. Lactam annulation was accomplished in 1% AcOH in DMSO with heating at 110° C. under microwave irradiation for 6 h, or in an oil bath for longer times. As previously reported on resin conversion to lactam was cleaner using the microwave; however, irradiation at 110° C. caused the lanterns to change morphology and degrade. Although lactam product could be recovered after cleavage from the altered lantern, it was observed that at 80° C., annulation could be effected without melting lantern and good conversion was achieved after 10 h.

In light of the improved activity of the (R)-Agl¹ and maintained potency of (R)-Agl² and (R)-Agl⁴ analogs of rytvela, the combinations of (R)-Agl¹-(R)-Agl² and (R)-Agl¹-(R)-Agl⁴ in rytvela analogs 96 and 97 were explored. For comparison, the (R)-Agl¹-D-Pro⁴ analog 95 was also synthesized. The three analogs were constructed using a split-and-pool approach on SynPhase-lanterns equipped with a Rink amide linker. The lanterns were equipped with coloured spindles as a visual tagging system. (R)-Cyclic sulfamidate (R)-8 was used to alkylate peptides bound to the lanterns suspended in THF at 60° C. for 1 h of microwave irradiation. Monitoring the reaction progress by LC-MS analyses after TFA-mediated cleavage of a lantern slice, it was found that, in contrast to synthesis on resin, the silylation with BSA had little effect on the amount of bis-alkylation product formed on lantern and comparable percentages of alkylated peptides 100 and 101 (27-30% and 50-52%, respectively) were achieved. This prompted the investigation of peptide alkylation without a prior BSA treatment. Longer heating in the microwave (2 h) at higher temperature (70° C.) and the addition of 1.1 equiv of DIEA to the sulfamidate solution increased the amount of 100 and 101 (>40% and >90%, respectively); however bis-alkylation of 98 was significant (50%).

Subsequently, the condition for lactam annulation on both 100 and 101 were optimized, and it was found that the addition of water to the DMSO (1% AcOH) solution gave, in both cases, quantitative lactam cyclization at 80° C. under microwave irradiation.

Investigating the feasibility of introducing a second lactam motif into the same peptide, alkylation and lactam annulation were performed using conditions previously effective for installing Agl residues: alkylation with (R)-8 (4 equiv) in THF (0.1 M) with microwave irradiation for 1 h at 70° C.; lactam formation in a solution of DMSO/AcOH/H₂O (75:2:23, v/v/v) with microwave irradiation for 10 h at 80° C. These conditions gave respectively 50, 8 and 26% conversions to 107, 108, 109. The addition of a second (R)-Agl residue presented a more difficult challenge than the first (R)-Agl residue. Different conditions were pursued to improve conversion in the second alkylation step including solvent, temperature and microwave irradiation time, without much success. The addition of 0.5 equiv of DIEA to the sulfamidate solution favoured alkylation. This amount of DIEA was chosen to avoid bis-alkylation, which was promoted with more base (1.1 equiv). Lactam formation by microwave irradiation at 80° C., Fmoc-deprotection and cleavage gave the crude peptides, which were analyzed by RP-HPLC to have 55, 30 and 45% crude purity for 95, 96 and 97, respectively (Table 9 and Scheme 11).

The efficacy of Agl peptides 93 and 94 was ascertained by measuring their influence on IL-1 induced human thymocyte TF-1 proliferation as assessed by incorporation of [³H]thymidine as in Example 59. The percentages of proliferation of TF-1 cells (150% and 132%, respectively) pre-treated with peptides 93 and 94 demonstrated that both these analogs lost inhibitory activity on TF-1 proliferation exhibited by rytvela (69%)^{xlixa}. In light of the fact that replacement of D-Thr by (S)— and (R)-Agl caused loss of activity, the side chain and conformation of this residue may both be essential for activity. As reported above, the (R)-Agl⁴ analog of rytvela exhibited similar activity as the parent peptide. On the contrary, here we find that the (S)-Agl⁴ diastereomer has lost activity indicative of the pronounced influence of configuration on peptide conformation and activity.

### Synthesis of (R)-Agl-D-Tyr-D-Thr-D-Val-(R)-Agl-D-Leu-D-Ala-NH2 (98)

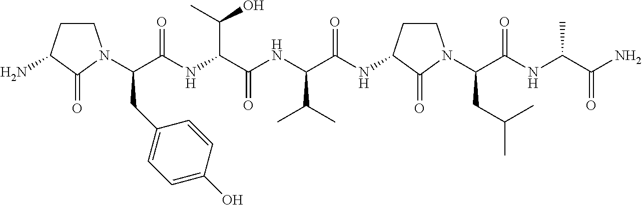

(R)-Agl-D-Tyr-D-Thr-D-Val-(R)-Agl-D-Leu-D-Ala-NH2 (98) was prepared on an A-sized polystyrene Rink amide lantern as previously mentioned for peptides 95 to 97. The crude peptide purity (30%) was assessed by RP-HPLC-MS (UV 214), 5-90% MeOH in H2O, 0.1% FA, 20 min gradient, tR 13.76 min., MS calcd. for C35H54N8O9 [M+H]+ 731.4. found 731.2).

### Example 61

### alpha-Amino-beta-hydroxy-gamma-lactams, Constrained Serine and Threonine Dipeptide Mimics

General Procedures

As previously mentioned for Examples 1-30.

### Synthesis

### (a). Synthesis of N-(Fmoc)Oxiranylglycine Reagent 106

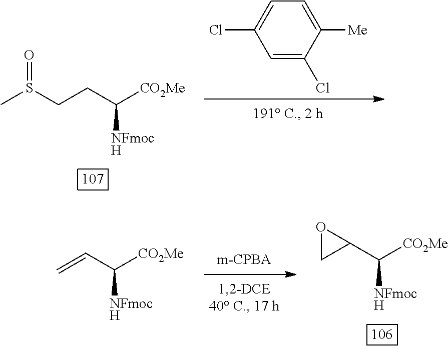

From known compound 107, an adapted literature procedure was followed:^{l}

### N-(Fmoc)Vinylglycine methyl ester

A 2,4-dichlorotoluene (30 mL) solution of N-(Fmoc)Met(S═O)—OMe 107 (4.01 g, 10.0 mmol) was heated to 191° C. for 2 h under Argon. The unconcentrated crude was loaded directly onto a 6.5 cm diameter×13 cm high pad of silica, which was eluted using a step gradient of 3→6→9→12→20→35→60% ethyl acetate:hexanes, 400 mL per step) yielding 2.81 g (84%) of N(Fmoc)Vinylglycine methyl ester.

### N-(Fmoc)Oxiranylglycine methyl ester (106)

N-(Fmoc)Vinylglycine methyl ester (1.69 g, 5.00 mmol) and m-CPBA (commercial ≦77%, 5.6 g, 25 mmol) were heated in 1,2-dichloroethane at 40° C. for 17 h. The crude was filtered over a frit, concentrated and purified by flash chromatography (7.5 cm wide×8.5 cm high silica pad, eluded by step gradient of 3→6→9→15→20→25% ethyl acetate:toluene, 400 mL per step) yielding 833 mg (47%) of N-(Fmoc)oxiranylglycine methyl ester.

### Representative Procedure for the Synthesis of β-hydroxy-α-amino-γ-lactams

D-Phe-OMe.HCl (77.6 mg, 360 mmol) in dilute Na₂CO₃ was extrated with CHCl₃ (3×1 mL), dried over MgSO₄, concentrated, and dried under high vacuum for a few hours to yield 40.8 mg (78%) of D-Phe-OMe. The free base (26.1 mg, 0.180 mmol) was immediately used in a reaction with N-(Fmoc)oxiranylglycine methyl ester 106 (18.4 mg, 0.052 mmol) at 76° C. in 2,2,2-trifluoroethanol (0.3 mL) for 15 h. The crude was concentrated in vacuo, and purified by flash chromatography (2.2 cm wide×2 cm high silica pad, step gradient of 28→31→34→37→50→70% ethyl acetate:hexanes, 20 mL per step) yielding 21.9 mg (91%) product (R_{f product}=0.2 with 60% ethyl acetate:hexanes eluent, R_{foxiranylglycine}=0.55).

### Spectroscopic Data

### (a) (2S)-[(9H-Fluoren-9-ylmethoxycarbonylamino)]-oxiranyl-acetic acid methyl ester (106)

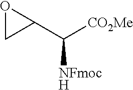

¹H NMR (400 MHz, CDCl₃) δ 7.77 (d, J=7.4 Hz, 2H), 7.60 (t, J=6.7 Hz, 2H), 7.41 (t, J=7.4 Hz, 2H), 7.32 (td, J=7.2, 2.5 Hz, 2H), 5.35 (d, J=9.0 Hz, 1H), 4.74 (d, J=8.9 Hz, 1H), 4.43 (d, J=7.1 Hz, 2H), 4.22 (t, J=6.8 Hz, 1H), 3.83 (s, 3H), 3.51-3.47 (m, 1H), 2.79 (t, J=4.3 Hz, 1H), 2.63 (dd, J=4.6, 2.6 Hz, 1H). ¹³C NMR (75 MHz, CDCl₃) 170.3, 156.3, 143.9, [143.7], 141.5₁, [141.4₇], 127.9, 127.2₇, [127.2₆], 125.2, 120.2, 76.8, 67.4, 53.2, 51.3, 47.3, 44.0. HRMS (ESI+) for MH⁺=C₂₀H₂₀NO₅⁺; calculated: 354.1336. found: 354.1331 (diff. m/z=1.3 ppm).

### (b) 2-[3-(9H-Fluoren-9-ylmethoxycarbonylamino)-4-hydroxy-2-oxo-pyrrolidin-1-yl]-propionic acid benzyl ester (108)

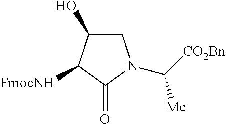

¹H NMR (300 MHz, CDCl₃) δ 7.78 (d, J=7.3 Hz, 2H), 7.59 (d, J=7.3 Hz, 2H), 7.46-7.29 (m, 9H), 5.70 (br s, 1H), 5.16 (dd, J=15.3, 12.2, 2H), 5.00-4.82 (m, 2H), 4.58-4.32 (m, 3H), 4.23 (t, J=7.0 Hz, 1H), 4.13 (pseudo t, J=7.2 Hz, 1H), 3.69 (t, J=3.69 Hz, 1H), 3.24 (t, J=8.7 Hz, 1H), 1.47 (d, J=7.2 Hz, 3H). ¹³C NMR (75 MHz, CDCl₃): δ (ppm) 170.6, 169.3, 158.2, 143.7, 141.5, 135.3, 128.9, 128.8, 128.4, 128.1, 127.3, 125.2, 120.3, 73.7, 67.9, 67.5, 60.7, 49.7, 47.6, 47.1, 15.1. HRMS (ESI⁺) for MH⁺=C₂₉H₂₉N₂O₆⁺; calculated: 501.2020. found: 501.2032 (error m/z=2.4 ppm).

### (c) 2-[3-(9H-Fluoren-9-ylmethoxycarbonylamino)-4-hydroxy-2-oxo-pyrrolidin-1-yl]-4-methyl-pentanoic acid methyl ester (109)

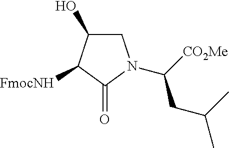

¹H NMR (400 MHz, CDCl₃): δ (ppm) 7.77 (d, J=7.5 Hz, 2H), 7.59 (d, J=7.5 Hz, 2H), 7.41 (t, J=7.5 Hz, 2H), 7.32 (t, J=7.4 Hz), 5.77 (br s, 1H), 4.88 (pseudo t, J=8.2 Hz, 1H), 4.44 (d, J=7.1 Hz, 2H), 4.29 (q, J=8.2 Hz, 1H), 4.22 (t, J=7.0 Hz, 1H), 4.13 (d, J=8.0 Hz, 1H), 3.72 (s, 1H), 3.56 (t, J=8.8 Hz, 1H), 3.38 (t. J=9.0 Hz, 1H), 1.83-1.35 (m, 3H), 1.01-0.87 (m, 6H). ESI⁺ for MH⁺=C₂₆H₃₁N₂O₆⁺ calculated and found: 467.2; for MNa⁺=C₂₆H₃₀N₂O₆Na⁺ calculated and found: 489.2.

### (d) [3-(9H-Fluoren-9-ylmethoxycarbonylamino)-4-hydroxy-2-oxo-pyrrolidin-1-yl]-acetic acid benzyl ester (110)

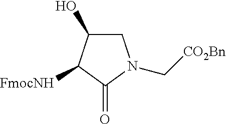

¹H NMR (300 MHz, CDCl₃): δ (ppm) 7.78 (d, J=7.5 Hz, 2H), 7.59 (d, J=7.4 Hz, 2H), 7.45-7.30 (m, 9H), 5.73 (s, 1H), 5.18 (s, 2H), 4.97 (very br), 4.48-4.36 (m, 3H), 4.28-4.07 (m, 4H), 3.63 (dd, J=9.4, 8.3 Hz, 1H), 3.44 (dd, J=9.3, 8.1 Hz, 1H). ESI⁺ for MH⁺=C₂₈H₂₇N₂O₆⁺ calculated and found: 487.2. For MNa⁺=C₂₈H₂₆N₂O₆Na⁺ calculated and found: 509.2.

### (e) 2-[3-(9H-Fluoren-9-ylmethoxycarbonylamino)-4-hydroxy-2-oxo-pyrrolidin-1-yl]-3-phenyl-propionic acid methyl ester (111)

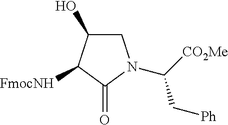

¹H NMR (300 MHz, CDCl₃): δ(ppm) 7.74 (d, J=7.5 Hz, 2H), 7.55 (d, J=7.2 Hz, 2H), 7.39 (t, J=7.3 Hz, 2H), 7.33-7.13 (m, 7H), 5.68 (s, 1H), 5.06 (dd, J=11.4, 4.9 Hz, 1H), 4.57-4.41 (m, 1H), 4.37 (d, J=7.1 Hz, 2H), 4.30 (q, J=8.1 Hz, 1H), 4.18 (t, J=6.9 Hz, 1H), 3.82 (dd, J=8.0, 1.8 Hz, 1H), 3.75 (s, 3H), 3.69 (dd, J=9.0, 8.0 Hz, 1H), 3.39 (dd, J=14.7, 5.0 Hz, 1H), 3.14 (t, J=3.4 Hz, 1H), 2.96 (dd, J=14.7, 11.5, 1H). ¹³C NMR (75 MHz, CDCl₃): δ (ppm) 170.3, 169.6, 158.2, 143.6_{4 [}143.5₆], 141.5 [141.4], 135.9, 129.0, 128.6, 128.0, 127.4, 127.2_{5 [}127.2₆], 125.1₄, [125.1₂], 120.2, 73.5, 67.8, 60.3, 55.1, 52.8, 48.0, 47.1, 35.4. ESI⁺ for C₂₉H₂₉N₂O₆⁺ calculated and found: 501.2. For C₂₉H₂₈N₂O₆Na⁺ calculated and found 523.2. HRMS (ESI⁺) for MH⁺=C₂₉H₂₉N₂O₆⁺ calculated: 501.2020. found: 501.2027 (diff. m/z=1.4 ppm).

### (f) 2-[3-(9H-Fluoren-9-ylmethoxycarbonylamino)-4-hydroxy-2-oxo-pyrrolidin-1-yl]-3-methyl-butyric acid methyl ester (112)

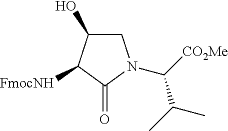

¹H NMR (700 MHz, CDCl₃): δ (ppm) 7.77 (d, J=7.7 Hz, 2H), 7.59 (dd, J=7.5, 4.0 Hz, 2H), 7.41 (t, J=7.5 Hz, 2H), 7.33 (tt, J=7.5, 1.1 Hz, 2H), 5.78 (s, 1H), 5.02 (br s, 1H), 4.52 (d, J=9.5 Hz, 1H), 4.44 (dd, J=10.7, 7.1, 1H), 4.42 (dd, J=10.7, 7.1, 1H), 4.36 (q, J=8.0 Hz, 1H), 4.23 (t, J=7.1 Hz, 1H), 4.14 (ddd, J=8.1, 1.9, 1.0 Hz, 1H), 4.03 (dd, J=9.5, 8.0 Hz, 1H), 3.73 (s, 3H), 3.25 (dd, J=9.6, 8.2 Hz, 1H), 2.23 (doublet of septuplets, J=9.5, 6.7 Hz, 1H), 0.99 (d, J=6.7 Hz, 3H), 0.96 (d, J=6.7 Hz, 3H). ¹³C NMR (100 MHz, CDCl₃): δ (ppm) 170.6, 169.8, 158.2, 143.7, 143.6, 141.5, 128.0, 127.3, 125.1, 120.2, 73.6, 67.9, 60.5, 59.9, 52.3, 48.0, 47.1, 28.1, 19.4, [19.3 (diasteriotopic Me)]. HRMS (ESI⁺) for MH⁺=C₂₆H₂₆N₂O₆⁺; calculated 453.2020. found: 453.2028 (diff. m/z=1.8 ppm).

### (g) 2-[3-(9H-Fluoren-9-ylmethoxycarbonylamino)-4-hydroxy-2-oxo-pyrrolidin-1-yl]-3-(4-hydroxy-phenyl)-propionic acid methyl ester (113)

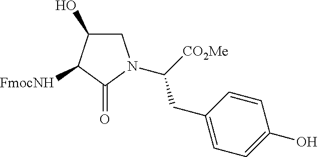

¹H NMR (300 MHz, CDCl₃): δ (ppm) 7.77 (d, J=7.6 Hz, 2H), 7.57 (d, J=7.6 Hz, 2H), 7.41 (t, J=7.6, 2H), 7.36 (br, 1H), 7.32 (t, J=6.8, 2H), 7.02 (d, J=8.3 Hz, 2H), 6.70 (d, J=8.4 Hz, 2H), 6.20 (very br s, 1H), 5.63 (br s, 1H), 5.09 (dd, J=11.7, 4.8 Hz, 1H), 4.45-2.48 (m, 3H), 4.20 (t, J=6.9 Hz, 1H), 3.92 (d, J=8.3 Hz, 1H), 3.77 (s, 3H), 3.83-3.68 (m, 1H), 3.31 (dd, J=11.5, 4.7 Hz, 1H), 3.15 (t, J=8.6 Hz, 1H), 2.88 (t, J=11.8 Hz, 1H). ESI⁺ for MH⁺=C₂₉H₂₆N₂O₇⁺ calculated and found: 517.2. For MNa⁺=C₂₆H₂₈N₂O₇Na⁺ calculated: 539.2. found: 539.3.

### (h) 2-[3-(9H-Fluoren-9-ylmethoxycarbonylamino)-4-hydroxy-2-oxo-pyrrolidin-1-yl]-3-(1H-indol-3-yl)-propionic acid methyl ester (114)

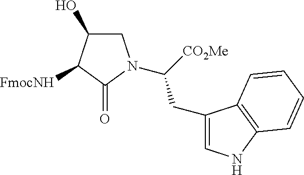

¹H NMR (300 MHz, CDCl₃): δ (ppm) 8.17 (br s, 1H), 7.77 (d, J=7.7 Hz, 2H), 7.62-7.53 (m, 3H), 7.45-7.10 (m, 7H), 7.00 Hz (br s, 1H), 5.65 (br s, 1H), 5.18 (dd, J=11.2, 4.7 Hz, 1H), 4.82 (very br s, 1H), 4.38-4.26 (m, 3H), 4.18 (t, J=6.9 Hz, 1H), 3.94 (dd, J=8.2, 2.4 Hz, 1H), 3.79 (s, 3H), 3.72 (t, J=8.6 Hz, 1H), 3.49 (dd, J=15.5, 4.7 Hz, 1H), 3.27-3.14 (m, 2H). ¹³C NMR (75.5 MHz, CDCl3): δ (ppm) 170.7, 169.7, 158.1, 143.7 [143.6], 141.4_{6 [}141.4₄], 136.3, 128.0, 127.3, 127.1, 125.1, 122.6, 122.0, 120.2, 119.9, 118.4, 111.5, 110.5, 73.5, 67.8, 60.4, 54.4, 52.8, 47.8, 47.1, 25.5. HRMS (ESI⁺) for MH⁺=C₃₁H₃₀N₃O₆⁺; calculated: 540.2129. found: 540.2141 (diff. m/z=2.2 ppm).

### (i) 3-[3-(9H-Fluoren-9-ylmethoxycarbonylamino)-4-hydroxy-2-oxo-pyrrolidin-1-yl]-propionic acid benzyl ester (115)

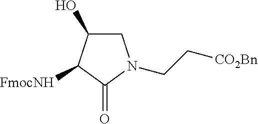

¹H NMR (300 MHz, CDCl₃): δ (ppm) 7.78 (d, J=7.5 Hz, 2H), 7.59 (d, J=7.3 Hz, 2H), 7.45-7.29 (m, 9H), 5.68 (br s, 1H), 5.13 (s, 2H), 4.87 (br s, 1H), 4.44 (d, J=6.8 Hz, 1H), 4.31-4.19 (m, 2H), 4.00 (d, J=8.0 Hz, 1H), 3.79-3.67 (m, 1H), 3.62-3.46 (m, 2H), 3.28 (t, J=8.8 Hz, 1H), 2.63 (t, J=6.6 Hz, 2H). ¹³C NMR (300 MHz, CDCl₃): δ (ppm) 171.1, 168.9, 158.2, 143.7, [143.6], 141.4₈, [141.4₇], 135.6, 128.8, 128.6, 128.0, 127.2_{7 [}127.2₆], 125.1₄, [125.1₁], 120.2₄, 120.2₂, 73.5, 67.8, 67.0, 60.8, 50.9, 47.1, 39.0, 32.5. HRMS (ESI⁺) for MH⁺=C₂₉H₂₉N₂O₆⁺; calculated: 501.2020. found: 501.2032 (diff. m/z=2.3).

### (j) 3-[3-(9H-Fluoren-9-ylmethoxycarbonylamino)-4-hydroxy-2-oxo-pyrrolidin-1-yl]-benzoic acid methyl ester (116)

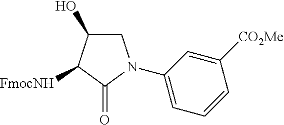

¹H NMR (300 MHz, CDCl₃): δ (ppm) 8.15 (pseudo t, J=1.9 Hz, 1H), 7.94 (ddd, J=8.1, 2.4, 1.0 Hz, 1H), 7.89 (dt, J=7.9, 1.3 Hz, 1H), 7.78 (d, J=7.6 Hz, 2H), 7.61 (d, J=7.5 Hz, 2H), 7.48 (t, J=8.0 Hz, 1H), 7.43 (t, J=7.3 Hz, 2H), 7.34 (td, J=7.5, 1.2 Hz, 2H), 5.84 (br s, 1H), 5.09 (br s, 1H), 4.54-4.42 (m, 3H), 4.31-4.21 (m, 2H), 4.08 (dd, J=9.8, 8.1 Hz, 1H), 3.94 (s, 3H), 3.79 (dd, J=9.6, 8.3 Hz, 1H). ESI⁺ for MH⁺=C₂₇H₂₅N₂O₆⁺; calculated and found: 473.2. For MNa⁺=C₂₇H₂₄N₂O₆Na⁺; calculated and found: 495.2.

Discussion

Enantiomerically pure (2S,3R)—N—(Cbz)-oxiranylglycine was prepared from L-Met according to literature procedures and examined in reactions with Ala-OBn. In acetonitrile at 90° C., the desired sequential alkylation/lactam formation occurred producing target β-hydroxy-α-amino-γ-lactam, albeit in 30% yield. The utility of Fmoc protection in peptide synthesis compelled further examination using N-(Fmoc)oxiranylglycine 106, which was prepared in an analogous manner from L-Met. Epoxide 106 reacted with Ala-OBn to produce lactam 108 in 10% yield. Little improvement was obtained in attempts to yield lactam 108 using Lewis and Brønsted acid catalysts. In the reaction between N-(Fmoc)Oxiranylglycine 106 and different amino acid analogs, 2,2,2-trifluoroethanol (TFE) as solvent proved optimum (Table 10).

For example, substrates with sterically demanding side chains such as the methyl esters of Phe, Val and Trp, reacted well with 106. In the case of Gly-OBn, lower reaction temperatures mitigated losses from Fmoc deprotection. The nucleophilic phenol of unprotected Tyr-OMe was tolerated, however the ester-protected glutamate side competed for lactamization producing N-alkyl pyroglutamate. In addition to proteinogenic examples, the methyl ester of aminobenzoic acid as well as the benzyl ester of beta-Ala gave respectively 58 and 67% yield.

The steriogenic carbons in these dipeptide mimics are derived from the chiral pool with diasteroselective induction to set the 3-hydroxy center by way of selective epoxidation of vinyl glycine. The oxiranylglycine diasteriomers 106 were separable, allowing all possible sterioisomers to be obtained by choice of chirality in the starting material.

### Preparation of beta-hydroxy-alpha-amino-gamma-lactam Peptides on Polystyrene Rink Amide Lantern

Synthesis of rytvela beta-hydroxy-alpha-amino-gamma-lactam analogue:

### D-Arg-D-Tyr-Agl(4-OH)-D-Val-D-Glu-D-Leu-D-Ala-NH2 (99)

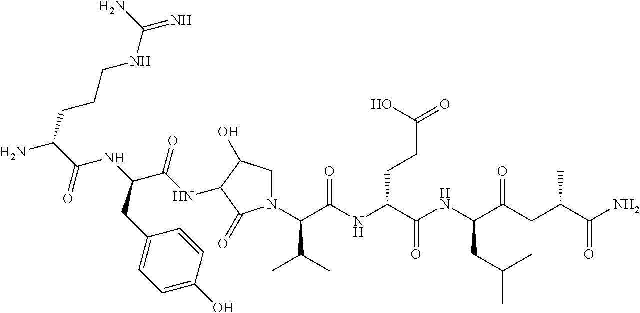

D-Arg-D-Tyr-Agl(4-OH)-D-Val-D-Glu-D-Leu-D-Ala-NH2 (99) was prepared on an A-sized polystyrene Rink amide lantern as previously mentioned for peptides 95 to 97 except the sulfamidate alkylation step was substituted by the following:

After Fmoc protecting group deprotection, the lanterns were treated with an oxiranylglycine 106 (3 equiv.) in 2,2,2-trifluoroethanol (0.06 M) and heated to 80° C. under microwave irradiation for 12 h. Lantern washing and subsequent elongation was done as described, providing the desired lactam peptide as the TFA salt 99 (50% crude purity by analytical RP-HPLC-MS (UV 214), 5-80% MeOH in H2O, 0.1% FA, 20 min gradient, tR 8.45 min., MS calcd. for C38H62O11N11 [M+H]+ 848.4. found 848.6.).

These scaffolds may find application in medicinal chemistry, especially because the hydroxy and protected amino groups are ideally suited for orthogonally elaborating these structures further. In the context of solid supported peptide synthesis, elaboration of the hydroxy group would allow for mimicry of other constrained amino acid residues, by the attachment of carbohydrates, phosphonate, sulfate and other ester and ether types.

TABLE 1

| | | | | HRMS | |
|---|---|---|---|---|---|
| | Crude | Purity | Yield | m/z | m/z |
| Peptide^{a} | Purity %^{b} | %^{c} | %^{c} | (calcd) | (obsd) |
| ( [symbol] )-Agl-D-Trp-Ala-Trp-D-Phe-Lys-NH₂ | 30 | >99 | 8 | 819.4300 | 819.4291 |
| 44 | | | | | |
| His-( [symbol] )-Agl-Ala-Trp-D-Phe-Lys-NH₂ | 36 | >99 | 22 | 385.7085 | 385.7094 |
| 45 | | | | | |
| His-D-Trp-( [symbol] )-Agl-Trp-D-Phe-Lys-NH₂ | 22 | >99 | 11 | 443.2304 | 443.2296 |
| 46 | | | | | |
| His-D-Trp-Ala-( [symbol] )-Agl-D-Phe-Lys-NH₂ | 66 | >99 | 11 | 770.4096 | 770.4090 |
| 47 | | | | | |
| His-D-Trp-Ala-Trp-( [symbol] )-Agl-Lys-NH₂ | 44 | >99 | 13 | 405.2139 | 405.2149 |
| 48 | | | | | |
| ( [symbol] )-Bgl-D-Trp-Ala-Trp-D-Phe-Lys-NH₂ | 39 | 92 | 22 | 819.4300 | 819.4298 |
| 49 | | | | | |
| His-( [symbol] )-Bgl-Ala-Trp-D-Phe-Lys-NH₂ | 26 | 91 | 12 | 770.4096 | 770.4097 |
| 50 | | | | | |
| His-D-Trp-( [symbol] )-Bgl-Trp-D-Phe-Lys-NH₂ | 46 | >99 | 25 | 885.4518 | 885.4511 |
| 51 | | | | | |
| His-D-Trp-Ala-( [symbol] )-Bgl-D-Phe-Lys-NH₂ | 52 | >99 | 6 | 770.4097 | 770.4089 |
| 52 | | | | | |
| His-D-Trp-Ala-Trp-( [symbol] )-Bgl-Lys-NH₂ | 92 | >99 | 5 | 809.4205 | 809.4198 |
| 53 | | | | | |

^{a}Bold lettering indicates lactam residues.
^{b}RP-HPLC purity at 214 nm of the crude peptide.
^{c}RP-HPLC purity at 214 nm of the purified peptide
^{d}Yields after purification by RP-HPLC are based on Fmoc loading test for Rink resin

TABLE 2

| | | | | HRMS | |
|---|---|---|---|---|---|
| | Crude | Purity | Yield | m/z | m/z |
| Peptide^{a} | Purity %^{b} | %^{c} | %^{d} | (calcd) | (obsd) |
| ( [symbol] )-Agl-D-Tyr-D-Thr-D-Val-D-Glu-D-Leu-D- | 55 | >99 | 19 | 777.4141 | 777.4138 |
| Ala-NH₂ | | | | | |
| 54 | | | | | |
| D-Arg-( [symbol] )-Agl-D-Thr-D-Val-D-Glu-D-Leu-D- | 41 | >99 | 15 | 770.4519 | 770.4514 |
| Ala-NH₂ | | | | | |
| 55 | | | | | |
| D-Arg-D-Tyr-( [symbol] )-Agl-D-Val-D-Glu-D-Leu-D- | 58 | >99 | 7 | 832.4675 | 832.4676 |
| Ala-NH₂ | | | | | |
| 56 | | | | | |
| D-Arg-D-Tyr-D-Thr-( [symbol] )-Agl-D-Glu-D-Leu-D- | 50 | >99 | 11 | 833.4468 | 833.4458 |
| Ala-NH₂ | | | | | |
| 57 | | | | | |
| D-Agl-D-Tyr-D-Thr-D-Val-( [symbol] )-Agl-D-Leu-D- | 36 | >99 | 21 | 803.4726 | 803.4724 |
| Ala-NH₂ | | | | | |
| 58 | | | | | |
| D-Arg-D-Tyr-D-Thr-D-Val-D-Glu-( [symbol] )-Agl-D- | 81 | >99 | 7 | 820.4311 | 820.4306 |
| Ala-NH₂ | | | | | |
| 59 | | | | | |

^{a}Bold lettering indicates lactam residue.
^{b}RP-HPLC purity at 214 nm of the crude peptide.
^{c}RP-HPLC purity at 214 nm of the purified peptide
^{d}Yields after purification by RP-HPLC are based on Fmoc

TABLE 3

| | | | | HRMS | |
|---|---|---|---|---|---|
| | Crude | Purity | Yield | m/z | m/z |
| Peptide^{a} | Purity %^{b} | %^{c} | %^{c} | (calcd) | (obsd) |
| ( [symbol] )-Agl-D-Trp-Ala-Trp-D-Phe-Lys-NH₂ | 19 | >93 | 14 | 819.4301 | 819.4308 |
| 60 | | | | | |
| His-( [symbol] )-Agl-Ala-Trp-D-Phe-Lys-NH₂ | 45 | >99 | 12 | 770.4096 | 770.4097 |
| 61 | | | | | |
| His-D-Trp-( [symbol] )-Agl-Trp-D-Phe-Lys-NH₂ | 30 | >98 | 4 | 443.2296 | 443.2301^{e} |
| 62 | | | | | |
| His-D-Trp-Ala-( [symbol] )-Agl-D-Phe-Lys-NH₂ | 61 | >99 | 11 | 770.4096 | 770.4097 |
| 63 | | | | | |
| His-D-Trp-Ala-Trp-( [symbol] )-Agl-Lys-NH₂ | 55 | >99 | 7 | 809.4205 | 809.4199 |
| 64 | | | | | |
| ( [symbol] )-Bgl-D-Trp-Ala-Trp-D-Phe-Lys-NH₂ | 30 | >97 | 5 | 819.4301 | 819.4307 |
| 65 | | | | | |
| Ac( [symbol] )-Bgl-D-Trp-Ala-Trp-D-Phe-Lys-NH₂ | 20 | >99 | 7 | 861.4406 | 861.4413 |
| 66 | | | | | |
| His-( [symbol] )-Bgl-Ala-Trp-D-Phe-Lys-NH₂ | 35^{f} | >99 | 8^{f} | 385.7085 | 385.7083^{e} |
| 67 | | | | | |
| His-D-Trp-( [symbol] )-Bgl-Trp-D-Phe-Lys-NH₂ | 21^{f} | >99 | 8^{f} | 443.2296 | 443.2301^{e} |
| 68 | | | | | |
| His-D-Trp-Ala-( [symbol] )-Bgl-D-Phe-Lys-NH₂ | 40 | >99 | 5 | 770.4096 | 770.4095 |
| 69 | | | | | |
| His-D-Trp-Ala-Trp-( [symbol] )-Bgl-Lys-NH₂ | 60 | >99 | 3 | 809.4205 | 809.4198 |
| 70 | | | | | |

^{a}Bold lettering indicates lactam residues.
^{b}RP-HPLC purity at 214 nm of the crude peptide (see conditions in experimental).
^{c}RP-HPLC purity at 214 nm of the purified peptide
^{d}Yields after purification by RP-HPLC are based on Fmoc loading test for Rink resin.
^{e}The dicharged cation [M + 2H]²⁺ was observed, theoretical mass was calculated accordingly.
^{f}using the optimized protocols described hereafter.

TABLE 4

| | | | IC₅₀ binding | IC₅₀ binding |
|---|---|---|---|---|
| Entry | Compound | Peptide | GHS-R1a | CD36 |
| 1 | GHRP-6 | His-D-Trp-Ala-Trp-D-Phe-Lys-NH₂ | 6.08 × 10⁻⁹ M | |
| 2 | Hexarelin | His-D-2-Me-Trp-Ala-Trp-D-Phe-Lys-NH₂ | | 3.33 × 10⁻⁶ M |
| 3 | 44 | ( [symbol] )-Agl-D-Trp-Ala-Trp-D-Phe-Lys-NH₂ | 4.09 × 10⁻⁸ M | 1.45 × 10⁻⁵ M |
| 4 | 45 | His-( [symbol] )-Agl-Ala-Trp-D-Phe-Lys-NH₂ | 2.86 × 10⁻⁵ M | 1.34 × 10⁻⁵ M |
| 5 | 46 | His-D-Trp-( [symbol] )-Agl-Trp-D-Phe-Lys-NH₂ | 2.39 × 10⁻⁶ M | 7.45 × 10⁻⁶ M |
| 6 | 47 | His-D-Trp-Ala-( [symbol] )-Agl-D-Phe-Lys-NH₂ | >>10⁻⁵ M | >>10⁻⁵ M |
| 7 | 48 | His-D-Trp-Ala-Trp-( [symbol] )-Agl-Lys-NH₂ | 3.10 × 10⁻⁶ M | 2.14 × 10⁻⁵ M |
| 8 | 49 | ( [symbol] )-Bgl-D-Trp-Ala-Trp-D-Phe-Lys-NH₂ | 3.71 × 10⁻⁷ M | >>10⁻⁵ M |
| 9 | 50 | His-( [symbol] )-Bgl-Ala-Trp-D-Phe-Lys-NH₂ | >>10⁻⁵ M | >>10⁻⁵ M |
| 10 | 51 | His-D-Trp-( [symbol] )-Bgl-Trp-D-Phe-Lys-NH₂ | 6.54 × 10⁻⁷ M | >>10⁻⁵ M |
| 11 | 52 | His-D-Trp-Ala-( [symbol] )-Bgl-D-Phe-Lys-NH₂ | >>10⁻⁵ M | >>10⁻⁵ M |
| 12 | 53 | His-D-Trp-Ala-Trp-( [symbol] )-Bgl-Lys-NH₂ | 2.45 × 10⁻⁵ M | 2.65 × 10⁻⁵ M |

TABLE 5

| | | | IC₅₀ binding | IC₅₀ binding |
|---|---|---|---|---|
| Entry | Compound | Peptide | GHS-R1a | CD36 |
| 1 | GHRP-6 | His-D-Trp-Ala-Trp-D-Phe-Lys-NH₂ | 6.08 × 10⁻⁹ M | |
| 2 | hexarelin | His-D-2-Me-Trp-Ala-Trp-D-Phe-Lys-NH₂ | | 3.33 × 10⁻⁶ M |
| 3 | 60 | ( [symbol] )-Agl-D-Trp-Ala-Trp-D-Phe-Lys-NH₂ | 3.46 × 10⁻⁶ M | 8.72 × 10⁻⁶ M |
| 4 | 61 | His-( [symbol] )-Agl-Ala-Trp-D-Phe-Lys-NH₂ | >>10⁻⁵ M | >>10⁻⁵ M |
| 5 | 62 | His-D-Trp-( [symbol] )-Agl-Trp-D-Phe-Lys-NH₂ | 2.82 × 10⁻⁶ M | 1.73 × 10⁻⁵ M |
| 6 | 63 | His-D-Trp-Ala-( [symbol] )-Agl-D-Phe-Lys-NH₂ | >>10⁻⁵ M | >>10⁻⁵ M |
| 7 | 64 | His-D-Trp-Ala-Trp-( [symbol] )-Agl-Lys-NH₂ | >>10⁻⁵ M | 3.82 × 10⁻⁵ M |
| 8 | 65 | ( [symbol] )-Bgl-D-Trp-Ala-Trp-D-Phe-Lys-NH₂ | | |
| 9 | 66 | Ac( [symbol] )-Bgl-D-Trp-Ala-Trp-D-Phe-Lys-NH₂ | non tested | |
| 10 | 67 | His-( [symbol] )-Bgl-Ala-Trp-D-Phe-Lys-NH₂ | | |
| 11 | 68 | His-D-Trp-( [symbol] )-Bgl-Trp-D-Phe-Lys-NH₂ | | |
| 12 | 69 | His-D-Trp-Ala-( [symbol] )-Bgl-D-Phe-Lys-NH₂ | >>10⁻⁵ M | 5.35 × 10⁻⁵ M |
| 13 | 70 | His-D-Trp-Ala-Trp-( [symbol] )-Bgl-Lys-NH₂ | >>10⁻⁵ M | >>10⁻⁵ M |

TABLE 6

| | | | | HRMS | |
|---|---|---|---|---|---|
| | Crude | Purity | Yield | m/z | m/z |
| Peptide^{a} | Purity %^{b} | %^{c} | %^{d} | (calcd) | (obsd) |
| ( [symbol] )-Bab-D-Tyr-D-Thr-D-Val-D-Glu-D-Leu-D- | 28 | >99 | 10 | 885.4714 | 885.4716 |
| Ala-NH₂ | | | | | |
| 75 | | | | | |
| D-Arg-( [symbol] )-Bab-D-Thr-D-Val-D-Glu-D-Leu-D- | 45 | >99 | 15 | 878.5092 | 878.5094 |
| Ala-NH₂ | | | | | |
| 76 | | | | | |
| D-Arg-D-Tyr-( [symbol] )-Bab-D-Val-D-Glu-D-Leu-D- | 27 | >99 | 3 | 940.5240 | 940.5251 |
| Ala-NH₂ | | | | | |
| 77 | | | | | |
| D-Arg-D-Tyr-D-Thr-( [symbol] )-Bab-D-Glu-D-Leu-D- | 6 | >99 | 4 | 942.5027 | 942.5043 |
| Ala-NH₂ | | | | | |
| 78 | | | | | |

^{a}Bold lettering indicates lactam residues.
^{b}RP-HPLC % at 214 nm of the crude mixture of the parent lactam peptide.
^{c}RP-HPLC purity at 214 nm of the purified peptide
^{d}Yields after purification by RP-HPLC are based on Fmoc loading test for Rink resin.

TABLE 7

| | Crude Purity | | | | |
|---|---|---|---|---|---|
| | %^{b} | | | HRMS | |
| | (lactamiza- | Purity | Yield | m/z | m/z |
| Petide^{a} | tion time) | %^{c} | %^{d} | (calcd) | (obsd) |
| ( [symbol] )-Bgl-D-Tyr-D-Thr-D-Val-D-Glu-D-Leu-D-Ala- | 71(4 h) | >99 | 22 | 777.4141 | 777.4145 |
| NH₂ | | | | | |
| 75 | | | | | |
| D-Arg-( [symbol] )-Bgl-D-Thr-D-Val-D-Glu-D-Leu-D-Ala- | 87(6 h) | >99 | 24 | 770.4519 | 770.4521 |
| NH₂ | | | | | |
| 76 | | | | | |
| D-Arg-D-Tyr-( [symbol] )-Bgl-D-Val-D-Glu-D-Leu-D-Ala- | 86(4 h) | >99 | 23 | 832.4676 | 832.4677 |
| NH₂ | | | | | |
| 77 | | | | | |
| D-Arg-D-Tyr-D-Thr-( [symbol] )-Bgl-D-Glu-D-Leu-D-Ala- | 79(7 h) | >99 | 17 | 833.4468 | 833.4470 |
| NH₂ | | | | | |
| 78 | | | | | |
| D-Bgl-D-Tyr-D-Thr-D-Val-( [symbol] )-Bgl-D-Leu-D-Ala- | 87(4 h) | >99 | 7 | 826.4546^{e} | 826.4544^{e} |
| NH₂ | | | | | |
| 79 | | | | | |
| D-Arg-D-Tyr-D-Thr-D-Val-D-Glu-( [symbol] )-Bgl-D-Ala- | 50(4 h) | >99 | 14 | 820.4312 | 820.4313 |
| NH₂ | | | | | |
| 80 | | | | | |
| ( [symbol] )-Bgl-D-Tyr-D-Thr-D-Val-D-Glu-D-Leu-D-Ala- | 70(10 h) | >99 | 13 | 777.4141 | 777.4138 |
| NH₂ | | | | | |
| 81 | | | | | |
| D-Arg-( [symbol] )-Bgl-D-Thr-D-Val-D-Glu-D-Leu-D-Ala- | 82(10 h) | >99 | 16 | 770.4519 | 770.4518 |
| NH₂ | | | | | |
| 82 | | | | | |
| D-Arg-D-Tyr-( [symbol] )-Bgl-D-Val-D-Glu-D-Leu-D-Ala- | 60(8 h) | >99 | 17 | 832.4676 | 832.4674 |
| NH₂ | | | | | |
| 83 | | | | | |
| D-Arg-D-Tyr-D-Thr-( [symbol] )-Bgl-D-Glu-D-Leu-D-Ala- | 87(7 h) | >99 | 14 | 833.4468 | 833.4469 |
| NH₂ | | | | | |
| 84 | | | | | |
| D-Bgl-D-Tyr-D-Thr-D-Val-( [symbol] )-Bgl-D-Leu-D-Ala- | 90(4 h) | >99 | 13 | 803.4726 | 803.4724 |
| NH₂ | | | | | |
| 85 | | | | | |
| D-Arg-D-Tyr-D-Thr-D-Val-D-Glu-( [symbol] )-Bgl-D-Ala- | 96(4 h) | >99 | 11 | 820.4131 | 820.4136 |
| NH₂ | | | | | |
| 86 | | | | | |

^{a}Bold lettering indicates lactam residues.
^{b}RP-HPLC purity at 214 nm of the crude peptide.
^{c}RP-HPLC purity at 214 nm of the purified peptide
^{d}Yields after purification by RP-HPLC are based on Fmoc loading test for Rink resin.
^{e}The cation [M + Na]⁺ was observed, theoretical mass was calculated accordingly.

TABLE 8

| | Crude | | | HRMS | |
|---|---|---|---|---|---|
| | Purity | Purity | Yield | m/z | m/z |
| Peptide^{a} | %^{b} | %^{c} | %^{d} | (calcd) | (obsd) |
| D-Arg-( [symbol] )-Bgl-D-Tyr-D-Thr-D-Val-D-Glu-D-Leu-D- | 56 | >99 | 18 | 933.5152 | 933.5152 |
| Ala-NH₂ | | | | | |
| 87 | | | | | |
| D-Arg-D-Tyr-( [symbol] )-Bgl-D-Thr-D-Val-D-Glu-D-Leu- | 55 | >99 | 6 | 933.5152 | 933.5137 |
| D-Ala-NH₂ | | | | | |
| 88 | | | | | |
| D-Arg-D-Tyr-D-Thr-( [symbol] )-Bgl-D-Val-D-Glu-D-Leu-D- | 71 | >99 | 10 | 933.5152 | 933.5152 |
| Ala-NH₂ | | | | | |
| 89 | | | | | |
| D-Arg-D-Tyr-D-Thr- D-Val-( [symbol] )-Bgl-D-Glu-D-Leu- | 68 | >99 | 6 | 933.5152 | 933.5147 |
| D-Ala-NH₂ | | | | | |
| 90 | | | | | |
| D-Bgl-D-Tyr-D-Thr-D-Val- D-Glu-( [symbol] )-Bgl-D-Leu- | 37 | >99 | 3 | 933.5152 | 933.5154 |
| D-Ala-NH₂ | | | | | |
| 91 | | | | | |
| D-Arg-D-Tyr-D-Thr-D-Val-D-Glu-D-Leu-( [symbol] )-Bgl-D- | 97 | >99 | 18 | 933.5152 | 933.5150 |
| Ala-NH₂ | | | | | |
| 92 | | | | | |

^{a}Bold lettering indicates lactam residues.
^{b}RP-HPLC purity at 214 nm of the crude peptide.
^{c}RP-HPLC purity at 214 nm of the purified peptide
^{d}Yields after purification by RP-HPLC are based on Fmoc loading test for Rink resin.
^{e}The cation [M + Na]⁺ was observed, theoretical mass was calculated accordingly.

TABLE 9

| | Crude | | | HRMS | |
|---|---|---|---|---|---|
| | Purity | Purity | Yield | m/z | m/z |
| Peptide | (%)^{a} | (%)^{b} | (%)^{c} | (calcd) | (obsd) |
| 93 D-Arg-D-Tyr-(S)-Agl-D-Val-D-Glu-D-Leu-D-Ala- | 32 | >99 | 15 | 832.4676 | 832.4678 |
| NH₂ | | | | | |
| 94 D-Arg-D-Tyr-D-Thr-(S)-Agl-D-Glu-D-Leu-D- | 18 | >99 | 5 | 834.4468 | 834.4459 |
| Ala-NH₂ | | | | | |
| 95 (R)-Agl-D-Tyr-D-Thr-D-Pro-D-Glu-D-Leu-D- | 55 | >95 | 10 | 775.3985 | 775.3986 |
| Ala-NH₂ | | | | | |
| 96 (R)-Agl-D-Tyr-D-Thr-(R)-Agl-D-Glu-D-Leu-D- | 30 | >99 | 5 | 761.3828 | 761.3832 |
| Ala-NH₂ | | | | | |
| 97 (R)-Agl-(R)-Agl-D-Thr-D-Val-D-Glu-D-Leu-D- | 45 | >65 | 5 | 697.3880 | 697.3901 |
| Ala-NH₂ | | | | | |

^{a}RP-HPLC purity at 214 nm of the crude peptide.
^{b}RP-HPLC purity at 214 nm of the purified peptide
^{c}Yields after purification by RP-HPLC are based on resin and lantern loading.
Bold lettering indicates lactam residues.

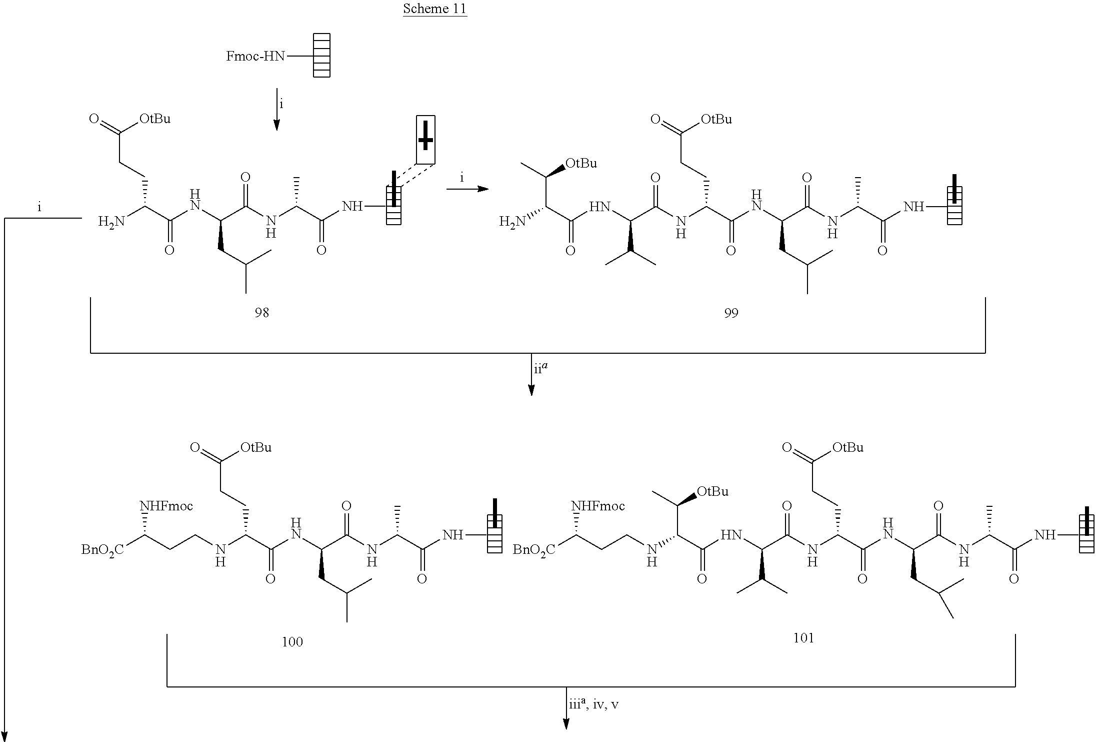

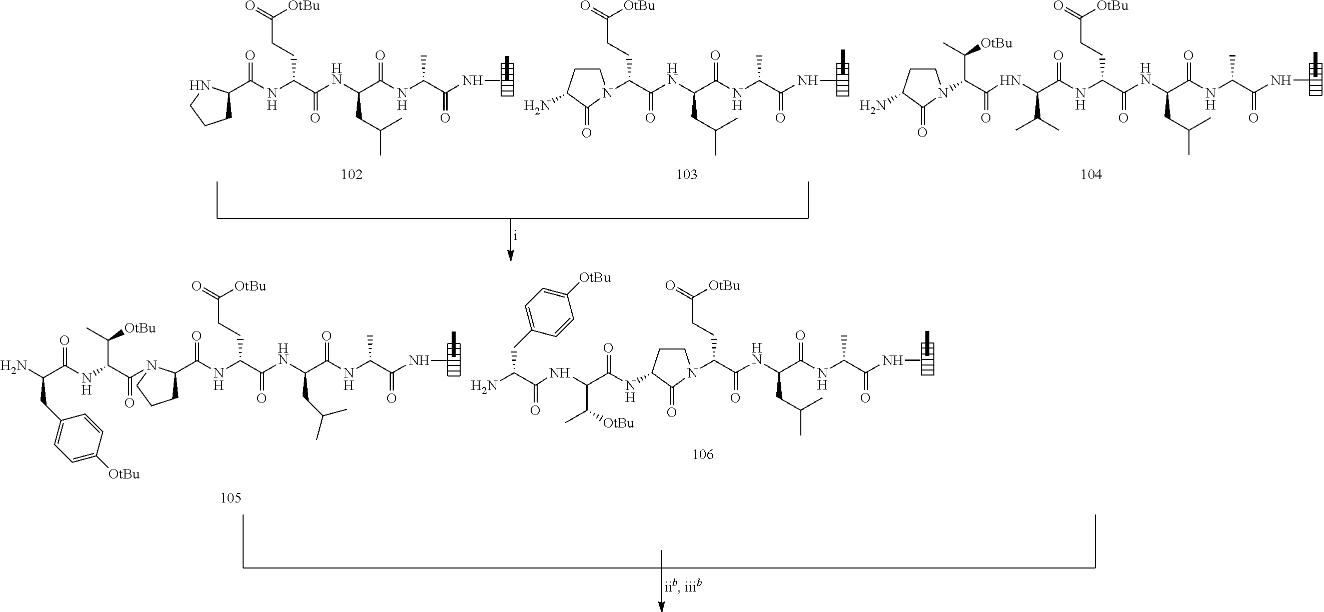

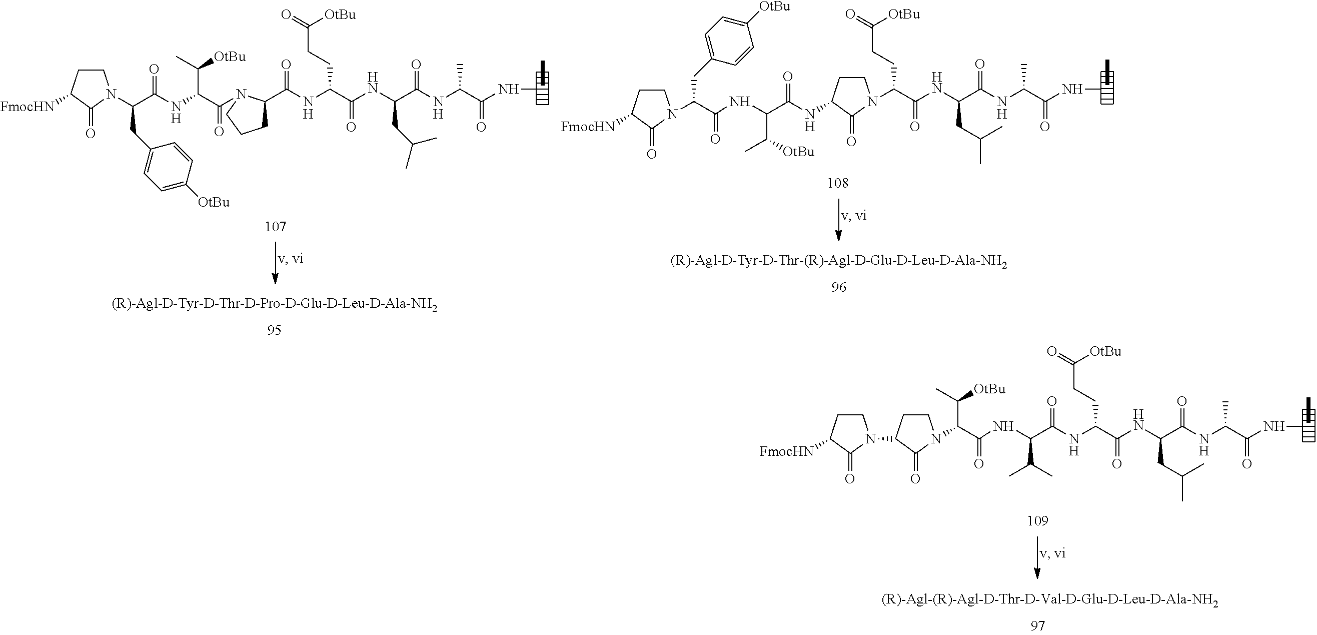

Split-mix synthesis of “rytvela” lactam-analogs 20, 21 and 22: (i) SPPS standard protocol; (iia) (R)-1 (4 eq), DIEA (1.1 equiv), THF, MW, 60-70° C., 2 h; (iiia) DMSO/H₂O/AcOH, MW, 80° C., 6-10 h; (iv) Ac2O/DIEA in DMF; (v) 20% piperidine/DMF; (iib) (R)-1 (4 eq), DIEA (0.5 equiv), THF, MW, 70° C., 2 h; (iiib) DMSO/H₂O/AcOH, MW, 80° C., 2×6 h; (vi) TFA/H2O/TES, 3 h.

TABLE 10

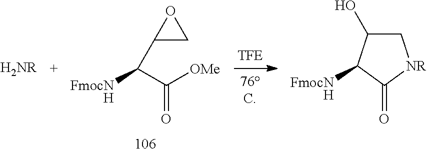

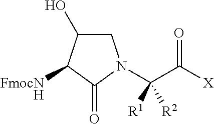

| | | | | % |
|---|---|---|---|---|
| | R¹ | R² | X | Yield^{a} |
| Gly-OBn | H | H | OBn | 49^{b} |
| Phe-OMe | H | Bn | OMe | 88 |
| D-Leu-OMe | ⁱBu | H | OMe | 91 |
| Val-OMe | H | ⁱPr | OMe | 90 |
| Glu(OMe)- | H | (CH₂)₂CO₂Me | OMe | 34^{c} |
| OMe | | | | |
| Trp-OMe | H | 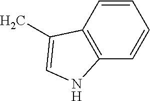 | OMe | 90 |
| Tyr-OMe | H | CH₂(p-HOPh) | OMe | 65 |

| | | |
|---|---|---|
| β-Ala-OMe | 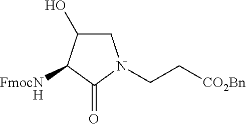 | 67^{b} |
| Aba-OMe | 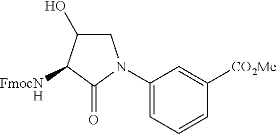 | 58 |

^{a}Isolated.
^{b}40° C.
^{b}Major product:

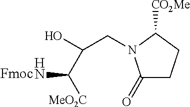

### FULL CITATIONS FOR DOCUMENTS REFERRED TO IN THE SPECIFICATION

ⁱ For selected reviews on peptidomimetic compounds, see (a) Seebach, D.; Gardiner J. Acc. Chem. Res., 2008, 41, 1366. (b) Chatterjee, J.; Gilon, C.; Hoffman, A.; Kessler, H. Acc. Chem. Res. 2008, 41, 1331. (c) Trabocchi, A.; Scarpi, D.; Guarna, A. Amino Acids 2008, 34, 1. (d) Vagner, J.; Qu, H.; Hruby, V. J. Current Opinion Chem. Bio. 2008, 12, 292. (e) Davis, J. M.; Tsou, L. K.; Hamilton, A. D. Chem. Soc. Rev. 2007, 36, 326. (f) Garner, J.; Harding, M. M. Org. Biomol. Chem. 2007, 5, 3577. (g) Cluzeau, J.; Lubell W. D. Biopolymers Pep. Sci, 2005, 80, 98. (h) Cowell, S. M.; Lee, Y. S.; Cain, J, P.; Hruby, V. J. Curr. Med. Chem. 2004, 11, 2785. (i) Loughlin, W. A.; Tyndall, J. D. A.; Glenn, M. P.; Fairlie, D. P. Chem. Rev. 2004, 104, 6085. (j) Giannis, A.; Rubsam, F. Peptidomimetics in Drug Design In Advances in Drug Research, Vol. 29; Testa, B.; Meyer, U. A. Eds.; Academic Press: San Diego; 1997, pp 1-78. (k) Hanessian, S.; McNaughton-Smith, G.; Lombart, H.-G.; Lubell, W. D. Tetrahedron 1997, 53, 12789. (l) Gante, J. Angew. Chem., Int. Ed. 1994, 33, 1699. (m) Wiley, R. A.; Rich, D. H. Med. Res. Rev. 1993, 13, 327. (n) Giannis, A.; Kolter, T. Angew. Chem., Int. Ed. 1993, 32, 1244.
ⁱⁱ Goodman, M.; Zhang, J. Chemtracts-Org. Chem. 1997, 10, 629.
ⁱⁱⁱ Gademann, K.; Ernst, M.; Hoyer, D.; Seebach, D. Angew. Chem., Int. Ed. 1999, 38, 1223.
^{iv} Stachowiak, K.; Khosla, M. C.; Plucinska, K.; Khairallah, P. A.; Bumpus, F. M. J. Med. Chem. 1979, 22, 1128.
^{v} (a) Hagihara, M.; Anthony, N. J.; Stout, T. J.; Clardy, J.; Schreiber, S. L. J. Am. Chem. Soc. 1992, 114, 6568. (b) Cho, C. Y.; Moran, E. J.; Cherry, S. R.; Stephans, J. C.; Fodor, S. P. A.; Adams, C. L.; Sundaram, A.; Jacobs, J. W.; Schultz, P. Science 1994, 261, 1303.
^{vi} Liskamp, R. M. J. Angew. Chem., Int. Ed. 1994, 33, 633.
^{vii} Burgess, K.; Linthicum, D. S.; Shin, H. Angew. Chem., Int. Ed. 1995, 34, 907.
^{viii} Han, H.; Janda, K. D. J. Am. Chem. Soc. 1996, 118, 2539.
^{ix} Han, H.; Yoon, J.; Janda, K. D. Methods Mol. Med. 1999, 23, 87.
^{x} Marshall, G. R. Tetrahedron 1993, 49, 3547.
^{xi} Gentilucci, L. Curr. Top. Med. Chem. 2004, 4, 19.
^{xii} Sagan, S.; Karoyan, P.; Lequin, O.; Chassaing, G.; Lavielle, S. Curr. Med. Chem. 2004, 11, 2799.
^{xiii} Reinelt, S.; Marti, M.; Dedier, S.; Reitinger, T.; Folkers, G.; Lopez de Castro, J. A.; Rognan, D. J. Bio. Chem. 2001, 276, 24525.
^{xiv} Taylor J. W. Biopolymers (Peptide Science) 2002, 66, 49.
^{xv} (a) Reichwein, J. F.; Wels, B.; Kruijtzer, J. A. W.; Versluis, C.; Liskamp, R. M. J. Angew. Chem. Int. Ed. 1999, 38, 3684. (b) Gilon, C.; Halle, D.; Chorev, M.; Selinger, Z.; Gerardo, B. Biopolymers 1991, 31, 745.
^{xvi} Melendez, R.; Lubell, W. D. J. Am. Chem. Soc. 2004, 126, 6759.
^{xvii} (a) Boeglin, D.; Lubell, W. D. J. Comb. Chem. 2005, 7, 864. (b) Boeglin, D.; Hamdan, F. F.; Melendez, R. E.; Cluzeau, J.; Laperriere, A.; Heroux, M.; Bouvier, M.; Lubell, W. D. J. Med. Chem. 2007, 50, 1401.
^{xviii} Freidinger, R. M.; Veber, D. F.; Perlow, D. S.; Brooks, J. R.; Saperstein, R. Science 1980, 210, 656.)
^{xix} Freidinger, R. M.; Perlow, D. S.; Veber, D. F. J. Org. Chem. 1982, 47, 104.
^{xx} Freidinger, R. M. J. Org. Chem. 1985, 50, 3631.
^{xxi} (a) Prdih A.; Kikelj D. Curr. Med. Chem. 2006, 13, 1525. (b) Aube, J. Synthetic Routes to Lactam Peptidomimetics In Advances in Amino Acid Mimetics and Peptidomimetics Vol. 1; Abell, A. Ed.; JAI Press Ltd: London; 1997, pp 193-232.
^{xxii} Ede, N. J.; Rae, I. D.; Hearn, M. T. W. Tetrahedron Lett. 1990, 31, 6071.
^{xxiii} Ede, N. J.; Lim, N.; Rae, I. D.; Ng, F. M.; Hearn, M. T. W. Peptide Res. 1991, 4, 171.
^{xxiv} Ede, N. J.; Rae, I. D.; Hearn, M. T. W. Aust. J. Chem. 1991, 44, 891.
^{xxv} Ede, N. J.; Rae, I. D.; Hearn, M. T. W. Int. J. Peptide Protein Res. 1994, 44, 568.
^{xxvi} Elliott, R. L.; Kopecka, H.; Tufano, M. D.; Shue, Y-K.; Gauri, A. J.; Lin, C-W.; Bianchi, B. R.; Miller, T. R.; Witte, D. G.; Stashko, M. A.; Asin, K. E. Nikkei, A. L.; Bednarz, L.; Nadzan, A. M. J. Med. Chem. 1994, 37, 1562.
^{xxvii} Bolbeare, K.; Pontoriero, G. F.; Gupta, S. K.; Mishra, R. K.; Johnson, R. L. Bioorg. Med. Chem. 2003, 11, 4103.

^{xxviii} examples include:—(a) Lang, M.; Söll, R. M.; Dürrenberger, F.; Dautzenberg, F. M.; Beck-Sickinger, A. G. J. Med. Chem. 2004, 47, 1153. (b) Krieger, F.; Möglich, A.; Kiefhaber, T. J. Am. Chem. Soc. 2005, 127, 3346. (c) Labro, A. J.; Raes, A. L.; Bellens, I.; Ottshytsch, N.; Snyders, D. J. J. Biol. Chem. 2003, 278, 50724. (d) Ceruso, M. A.; McComsey, D. F.; Leo, G. C.; Andrade-Gordon, P.; Addo, M. F.; Scarborough, R. M.; Oksenberg, D.; Maryanoff, B. E. Bioorg. Med. Chem. 1999, 7, 2353.)
^{xxix} Toniolo, C. Int. J. Pept. Protein Res. 1990, 35, 287, and references therein.
^{xxx} Thompson, P. E.; Lim, N.; Ede, N. J.; Ng, F. M.; Rae, I. D.; Hearn, M. T. W. Drug Design and Discovery 1995, 13, 55.
^{xxxi} Higgins, K. A.; Thompson, P. E.; Hearn, M. T. W. Int. J. Peptide Protein Res. 1996, 48, 1.
^{xxxii} (a) Wolf, J-P.; Rapoport, H. J. Org. Chem. 1989, 54, 3164. (b) Schuster, M.; Blechert, S. Angew. Chem. Int. Ed. 1997, 36, 2036.
^{xxxiii} (a) Wolfe, M. S.; Dutta, D.; Aubé, J. J. Org. Chem. 1997, 62, 654. (b) Nöth, J.; Frankowski, K. J.; Neuenwander, B.; Aubé, J. J. Comb. Chem. 2008, 10, 456.
^{xxxiv} Piscopio, A. P. D.; Miller J. F.; Koch K. Tetrahedron Letters. 1998, 39, 2667.
^{xxxv} Armstrong, S. K. J. Chem. Soc., Perkin Trans. 1 1998, 1, 371.
^{xxxvi} Piscopio, A. D.; Miller, J. F.; Koch K. Tetrahedron 1999, 55, 8189.
^{xxxvii} Galaud, F.; Lubell, W. D. Biopolymers (Peptide Science) 2005, 80, 665.
^{xxxviii} Bhooma, R.; Rodney, J. J. Org. Chem. 2006, 71, 2151.
^{xxxix} Freidinger, R. M. J. Med. Chem. 2003, 46, 5553.
^{xl} Scott, W. L.; Alsina, J.; Kennedy, J. H.; O'Donnell, M. J. Org. Lett. 2004, 6, 1629.
^{xli} Lama, T.; Campiglia, P.; Carotenuto, A.; Auriemma, L.; Gomez-Monterrey, I.; Novellino, E.; Grieco, P. J. Peptide Res. 2005, 66, 231.
^{xlii} Feytens, D.; De Vlaeminck, M.; Tourwé, D. J. Pept. Sci. 2009, 15, 16.
^{xliii} Boyarskaya, N. P.; Prokhorov, D. I.; Kirillova, Y. G.; Zvonkova, E. N.; Shvets, V. Tetrahedron Lett. 2005, 46, 7359.
^{xliv} Melendez, R.; Lubell, W. D. Tetrahedron 2003, 3, 2965 and references therein.
^{xlv} Galaud, F.; Blankenship, J. W.; Lubell, W. D. Heterocycles 2008, 76, 1121 and references therein.
^{xlvi} Galaud, F.; Demers, A.; Ong, H.; Lubell, W. D. In Understanding Biology Using Peptides Proceedings of the 19th American Peptide Symposium; Blondelle, S. E. Ed.; Springer: New York; 2005, pp 188-189.
^{xlvii} Boeglin, D. R., Bodas, M. S., Galaud, F., Lubell, W. D. In Understanding Biology Using Peptides: Proceedings of the 19th American Peptide Symposium; Blondelle, S. E. Ed.; Springer: New York; 2005, pp 47-48.
^{xlviii} Bodas, M.; Lev-Goldman, V.; Ben-Aroya, N.; Koch, Y.; Fridkin, M.; Lubell, W. D., Peptides 2006: Proceedings of 29th European Peptide Symposium; Rolka, K., Rekowski, P., Silberring, J. eds.; 2006, pp 261-262.
^{xlix} (a) Quiniou, C.; Przemyslaw, S.; Lahaie, I.; Hou, X.; Brault, S.; Beauchamp, M.; Leduc, M.; Rihakova, L.; Joyal, J-S.; Nadeau, S.; Heveker, N.; Lubell, W. Sennlaub, F.; Gobeil, Jr., F.; Miller, G.; Pshezhtsky, A. V.; Chemtob, S. J. Immunol. 2008, 180, 6977. (b) Chemtob, S.; Quiniou, C.; Lubell, W. D.; Beauchamp, M.; Hansford, K. A. 2006, US Patent No. 20,060,094,663.
^{l} (a) Organ, M., G.; Xu, J.; N'Zemba, B. Tetrahedron Lett. 2002, 43, 8177; (b) Original N-(Cbz)Vinylglycine-OMe synthesis: Afzali-Ardakani, A.; Rapoport, H. J. Org. Chem. 1980, 45, 4817; Carrasco, M.; Jones, R., J.; Kamel, S.; Rapoport, H. Organic Syntheses, Coll. Vol. 9, p. 63 (1998); Vol. 70, p. 29 (1992), (c) Meffre, P.; Vo-Quang, L.; Vo-Quang, Y.; Le Goffic, F. Synthetic Communications 1989, 19, 3457.
^{li} (a) Boyle, P. H.; Davis A. P.; Dempsey, K. J.; Hosken, G. D. Tetrahedron: Asymmetry 1995, 6, 2819. (b) Shiraiwa, T.; Nakagawa, K.; Kanemoto, N.; Kinda, T.; Yamamoto, H. Chem. Pharm. Bull. 2002, 50, 1081.
^{lii} Posakony, J. J.; Grierson, J. R.; Tewson, T. J. J. Org. Chem. 2002, 67, 5164.
^{liii} Carlsen, P. H. J.; Katsuki, T.; Martin, V. S.; Sharpless, K. B. J. Org. Chem. 1981, 46, 3936.
^{liv} Gmeiner, P.; Feldman, P. L.; Chu-Moyer, M. Y.; Rapoport, H. J. Org. Chem. 1990, 55, 3068.
^{lv} Booth, S.; Wallace, E. N. K.; Singhal, K.; Bartlett P. N.; Kilburn, J. D. J. Chem. Soc., Perkin Trans. 11998, 1467.
^{lvi} Grimm, E. L.; Roy, B., Aspiotis, R.; Bayl), C. I.; Nicholson, D. W.; Rasper, D. M.; Renaud, J.; Roy, S.; Tam, J., Tawa, P.; Vaillancourt, J. P.; Xanthoudakis S.; Zamboni, R. J. Bioorg. Med. Chem. 2004, 12, 845.
^{lvii} Still, W. C.; Kahn, M.; Mitra, A. J. Org. Chem. 1978, 43, 2923.
^{lviii} Filira, F., Biondi, B., Biondi, L., Giannini, E., Gobbo, M., Negri, L., Rocchi, R., Org. Biomol. Chem., 2003, 1, 3059.
^{lix} Kaiser, E.; Colescott, R. L.; Bossinger C. D.; Cook P. I. Anal. Biochem. 1970, 34, 595.
^{lx} Fukuyama, T.; Jow, C-K. Tetrahedron Lett. 1995, 36, 6373.
^{lxi} (a) Miller, S. C.; Scanlan, T. S. J. Am. Chem. Soc. 1997, 119, 2301. (b) Miller, S. C.; Scanlan, T. S. J. Am. Chem. Soc. 1998, 120, 2690. (c) Biron, E.; Chatterjee, J.; Kessler, H. J. Peptide Sci. 2006, 12, 213.
^{lxii} Reichwein, J. F.; Liskamp, M. J. Tetrahedron Lett. 1998, 39, 1243. (b) Yang, L.; Chiu, K. Tetrahedron Lett. 1997, 38, 7307.
^{lxii} Lin, X.; Dorr, H.; Nuss, J. M. Tetrahedron Lett. 2000, 41, 3309.
^{lxiv} (a) Fields G. B.; Noble R. L. Int. J. Pept. Protein Res. 1990, 35, 161. (b) Lubell, W. D.; Blankenship, J. W.; Fridkin, G.; Kaul, R. 21.11 Peptides. In Science of Synthesis, Volume 21: Three Carbon-Heteroatom Bonds: Amides and Derivatives; Peptides; Lactams.; Weinreb, S. M. Ed.; Thieme: Stuttgart, 2005.
^{lxv} www.irori.com
^{lxvi} Harigaya, S.; Schwartz, A. Circ. Res. 1969, 25, 781.
^{lxvii} Ong, H.; McNicoll, N.; Escher, E.; Collu, R.; Deghenghi, R.; Locatelli, V.; Ghigo, E.; Muccioli, G.; Boghen, M.; Nilsson, M. Endocrynology 1998, 129, 432.
^{lxviii} Muccioli, G; Papotti, M; Locatelli, V; Ghigo, E; Deghenghi, R. J. of Endocrinol. Invest. 2001, 24, RC7.

1. A process for the preparation of alpha-amino-gamma-lactams or beta-amino-gamma-lactams comprising:
(a) reacting compounds of the Formula (I): 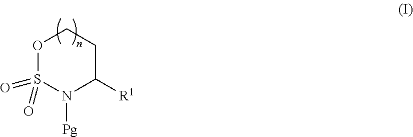
wherein Pg is a suitable protecting group,
n is 0 or 1,
when n is 1, R¹ is CO₂Bn, and
when n is 0, R¹ is selected from CH₂CO₂C₁₋₆alkyl and CH₂CO₂Bn,
with suitable amines of the Formula II: 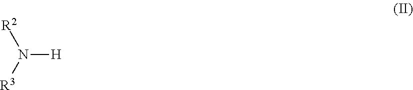
wherein R² is a hydrocarbon-based group comprising acyclic, cyclic, branched, unbranched, saturated, unsaturated and/or aromatic moieties that are unsubstituted or substituted with hetereomoieties, and
R³ is selected from H and a suitable protecting group,
under conditions to form compounds of the Formula (III): 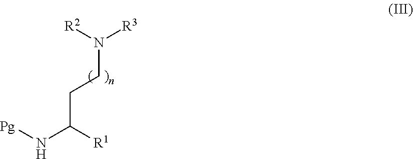
wherein Pg, R¹ and n are as defined in Formula (I), and
R² and R³ are as defined in Formula (II);
(b) if R³ is a suitable protecting group, removing R³;
(c) treating the compounds of the Formula (III), wherein R³ is H, under conditions to form alpha-amino-gamma-lactams or beta-amino-gamma-lactams of the Formula (IV): 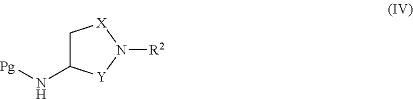
wherein Pg is as defined in Formula (I),
R² is as defined in Formula II, and
X is C(O) and Y is CH₂ when n in the compounds of Formula (I) is 0 and X is CH₂ and Y is C(O) when n in the compounds of Formula (I) is 1; and
(d) optionally removing Pg.

2. The process of claim 1, wherein the amines of Formula (II) are selected from one of the proteinogenic amino acids.

3. The process of claim 2, wherein the proteinogenic amino acids comprise side group functionalities in protected form.

4. The process of claim 1, further comprising, following (c), reacting the compounds of the Formula (III) to replace any protecting groups that were present on any functional groups in R² that were removed during (a) and/or (c).

5. The process of claim 1, wherein the amines of Formula (II) are attached to a solid support.

6. The process of claim 1, wherein the conditions to form compounds of the Formula (III) comprise reacting excess amounts of the compounds of Formula (I) with the amines of Formula (II) in a suitable organic solvent, at temperatures in the range of about 20° C. to about 70° C., for about 1 hour to about 48 hours.

7. The process of claim 6, wherein the conditions to form compounds of the Formula (III) further comprise reacting excess amounts of the compounds of Formula (I) with the amines of Formula (II) in presence of microwave irradiation.

8. (canceled)

9. The process of claim 1, wherein R³ is H and the conditions to form compounds of the Formula (III) comprise reacting the compounds of the Formula (I) with the compounds of the Formula (II) in the presence 1 equivalent or less of a non-nucleophilic base.

10. The process of claim 1, wherein the conditions to form compounds of the Formula (IV) comprise treating the compounds of the Formula (III) wherein R³ is H in a suitable organic solvent, in the presence of microwave irradiation at a temperature of about 60° C. to about 120° C. for about 10 minutes to about 24 hours, suitably about 30 minutes to about 10 hours.

11-13. (canceled)

14. The process of claim 1, wherein R³ is H and (a) and (c) are performed in a single step and the conditions to form the compounds of the Formula (III) are sufficient to form the compounds of the Formula (IV) so that upon formation of the compound of the Formula (III) it is converted, in situ, into the compounds of the Formula (IV).

15. The process of claim 14, wherein the conditions comprise microwave irradiation.

16. The process of claim 1, wherein the compounds of the Formula (III) are isolated and Pg is removed to provide a compound of the Formula (V): 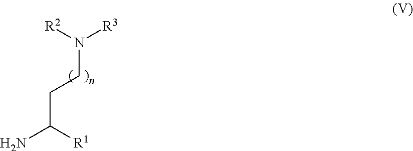
wherein R¹ and n are as defined in Formula (I) and R² and R³ are as defined in Formula (II). (V)

17. The process of claim 1, wherein the process is performed on a solid support using standard peptide synthesis procedures to insert one or more alpha-amino-gamma-lactam and/or beta-amino-gamma-lactam groups into a peptide.

18-19. (canceled)

20. A process for the preparation of beta-hydroxy-alpha-amino-gamma-lactams comprising:
(a) reacting compounds of Formula (VI): 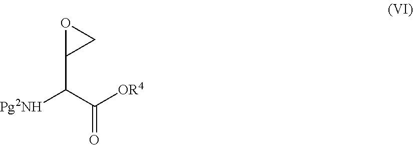
wherein Pg² is a suitable protecting group, and
R⁴ is selected from C₁₋₆alkyl and Bn,
with amines of the Formula (VII):
R⁵—NH₂ (VII)
wherein R⁵ is a hydrocarbon-based group comprising acyclic, cyclic, branched, unbranched, saturated, unsaturated and/or aromatic moieties that are unsubstituted or substituted with hetereomoieties,
under conditions to form compounds of the Formula (VIII): 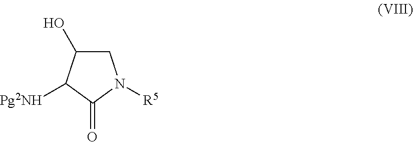
wherein Pg² is as defined in Formula (VI) and R⁵ is as defined in Formula (VII); and
(b) optionally removing Pg².

21. The process of claim 20, wherein the amines of Formula (VII) are selected from one of the proteinogenic amino acids.

22. The process of claim 21, wherein the proteinogenic amino acids comprise side group functionalities in protected form.

23. The process of claim 22 wherein (b) further comprises removing side group protecting groups on the amines of Formula (VI).

24. The process of claim 20, wherein the amines of Formula (VII) are attached to a solid support.

25-26. (canceled)

27. A compound of the Formula (I): 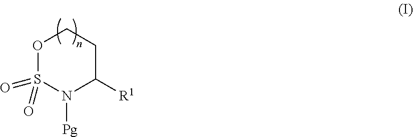
wherein Pg is a suitable protecting group,
n is 0 or 1,
when n is 1, R¹ is CO₂Bn, and
when n is 0, R¹ is selected from CH₂CO₂C₁₋₆alkyl and CH₂CO₂Bn.

28. The compound of claim 27, wherein Pg is Fmoc or t-Boc.

29.-31. (canceled)
